(19) United States
(12) Patent Application Publication
(10) Pub. No.: US 2023/0346989 A1
(43) Pub. Date: Nov. 2, 2023

(54) FLUOROCARBON NANOEMULSIONS AND USES THEREOF IN IMAGING

(71) Applicant: The Regents of the University of California, Oakland, CA (US)

(72) Inventors: Eric T. Ahrens, Encinitas, CA (US); Stephen Adams, Poway, CA (US); Chao Wang, San Diego, CA (US); Benjamin Leach, Del Mar, CA (US)

(21) Appl. No.: 18/026,759

(22) PCT Filed: Sep. 17, 2021
(86) PCT No.: PCT/US2021/071498

§ 371 Date: Mar. 16, 2023

Related U.S. Application Data
(60) Provisional application No. 63/080,525, filed on Sep. 18, 2020.

(51) Int. Cl. A61K 51/04; A61K 51/12

(52) U.S. Cl. CPC A61K 51/0497; A61K 51/0478; A61K 51/122; A61K 2123/00

(57) ABSTRACT

The disclosure relates to compositions comprising (i) at least one fluorocarbon, (ii) at least one fluorous hydroxamic acid chelator, and in some instances, (iii) at least one radioisotope. The disclosure also relates to methods for using such compositions, methods of making such compositions, and kits for making such compositions.

### CROSS-REFERENCE TO RELATED APPLICATIONS

This application claims the benefit of U.S. Appl. Ser. No. 63/080,525, filed Sep. 18, 2020, which is incorporated by reference as if fully set forth herein.

### STATEMENT OF GOVERNMENT SUPPORT

This invention was made with government support under grant CA134633 and EB024015 awarded by the National Institutes of Health. The government has certain rights in the invention.

### BACKGROUND

Inflammation is a defensive innate immune response toward invasive stimuli and features activation and recruitment of immune cells. Though it beneficially promotes pathogen clearance and tissue recovery, uncontrolled inflammatory responses drive disease pathobiology. While conventional tissue contrast-based imaging methods, including proton magnetic resonance imaging (MRI) and computed tomography (CT), detect non-cell-specific inflammation lesions at late stage, molecular imaging methods offer the potential for increased specificity, earlier diagnosis, and improved therapeutic outcomes. Thus, there is keen interest in developing molecular imaging probes for nuclear imaging and MRI with precise targeting to inflammatory cells and markers.

The positron emission tomography (PET) probe fluorodeoxyglucose (FDG), a glucose analog labeled with fluorine-18 (t_{1/2}=108 min), serves as an imaging biomarker for numerous inflammatory diseases. However, FDG can have prominent uptake in tissues such as heart, kidney and gastrointestinal tract that may confound image interpretation for diseases affecting these regions. In the case of solid tumor and metastasis, it is challenging to distinguish between tumor associated macrophages (TAMs) and dividing tumor cells in FDG PET scans, as both are metabolically active and take up the agent. To improve inflammation specificity, macrophage uptake of ¹⁸F-mannose has been visualized by PET. Other small molecule radiotracers targeting inflammatory markers, such as cytokines, translocator proteins, enzymes, and integrin receptors, have been designed for enhanced specificity with varying degrees of success. Nanoparticle PET tracers for macrophages have also been explored, including ¹⁸F and ⁶⁴Cu polyglucose and ⁸⁹Zr dextran.

Given the high endocytic activity of monocytes and macrophages and their heavy involvement in inflammatory processes, nanometer-sized probes mimicking ‘pathogens’ are a powerful cell delivery approach, exploiting highly-evolved cell functions for efficient intracellular probe labeling in situ. Intravenously administered fluorocarbon nanoemulsions enable background-free ‘hotspot’ fluorine-19 MRI (FMRI) detection. The nanoemulsion droplets are scavenged in situ by cells of the reticuloendothelial system (RES), particularly monocytes, macrophages, but also neutrophils and dendritic cells. The fluorous droplets can coalesce into phagocyte lysosomal vesicles and macropinosomes, thus escaping osmotic pressure based cell efflux and yields durable labeling, in contrast to small molecule tracers. Fluorocarbons have a proven safety profile and a well-characterized biodistribution and pharmacokinetics. The biological inertness and high oxygen solubility of fluorocarbons have made them major candidates for oxygen-carrying blood substitutes since the 1980s. In the clinic, microbubbles made from fluorocarbons are routinely used for contrast-enhanced ultrasound imaging. Moreover, clinical immunotherapeutic cells, pre-labeled with fluorocarbon nanoemulsion, have been longitudinally imaged with FMRI post-inoculation into cancer patients.

### SUMMARY

The disclosure generally relates to compounds for sensitive and precise inflammation imaging using PET and FMRI using functionalized fluorocarbon nanoemulsions (˜160 nm droplet size) to incorporate a fluorous phase-encapsulated radiometal chelate (FERM) that captures ⁸⁹Zr into the fluorous phase of the preformed nanoemulsion via a simple premix step. Fluorous encapsulation of ⁸⁹Zr can exclude bulk water, resulting in a highly stable complex that minimizes demetallation of FERM nanoemulsion. Using FERM, effective detection of macrophage-associated inflammation using multimodal PET-FMRI has been demonstrated in murine models of acute infection, IBD and breast cancer. Results display robust whole-body lesion detection that leverages the strengths of PET (sensitivity), FMRI (low background), and ¹H MRI and CT (high resolution anatomical localization). PET using FERM nanoemulsion is a departure from conventional small-molecule and recombinant protein PET probe approaches for inflammation detection, offering simplicity and highly specific targeting of phagocytic immune cells in vivo. FERM nanoemulsion production is scalable and potentially translatable for precise diagnostic monitoring of inflammatory processes.

### DESCRIPTION OF THE DRAWINGS

FIG. 1A is a scheme showing the design and characterization of FERM nanoemulsion for PET-FMRI imaging as described herein. The experimental workflow, including processes for emulsification, radiolabeling, injection and in situ macrophage labeling

FIG. 1B is a scheme showing the synthesis and structure of FHOA.

FIG. 1C is a simulated complex between FHOA and Zr⁴⁺.

FIGS. 1D-1E are ¹H and ¹⁹F NMR spectra, respectively, showing reaction products between FHOA and ZrCl₄ (non-radioactive) at varying doses in CD₃OD.

FIG. 1F is UV-Vis spectra of a nanoemulsion with addition of Zr⁴⁺ (2eq) or Fe³⁺ (2eq). Adding Zr⁴⁺ to the Fe-bound FERM nanoemulsion (red line) causes a decrease in absorbance at ˜450 nm (grey line). “Control” is FERM nanoemulsion without metal (black line).

FIG. 1G is a plot of UV-Vis absorbance at 450 nm over time after adding Zr⁴⁺ (2 eq) to Fe-bound FERM nanoemulsion; a bi-exponential fit yields t_{1/2} values of 0.32±0.07 h and 2.91±0.26 h, with R²=0.9971.

FIG. 1H is bar graphs showing the ¹⁹F T₁ of PFOB (—CF₂Br peak) nanoemulsion upon metal binding, where addition of Fe³⁺ (2 eq) decreases T₁. Addition of Zr⁴⁺ [ZrCl₄ or Zr oxalate, 2 eq] to the Fe³⁺ bound nanoemulsion partially reverses the effect of Fe³⁺. Addition of excess Fe³⁺ to Zr-bound emulsion has minimal effect on T₁. Data are mean±s.e.m. (n=3), where *** denotes P<0.001 for unpaired t-tests.

FIGS. 2A-2C are plots from a pharmacokinetic blood analysis of FERM nanoemulsion in mouse showing agent stability in vivo. FIG. 2A displays decay-corrected gamma counts of blood samples (100 μL, n=3 mice per time point) drawn over a 24 h period. Assuming a bi-exponential decay fit, the median circulation half-lives are 0.6(0.2) h for the fast component and 14.5(0.07) h for slow phase. In FIG. 2B, the fluorine content of the same blood samples from FIG. 2A, measured by ¹⁹F NMR, shows bi-exponential behavior with 0.9(0.006) h and 14.6(0.11) h for the fast and slow components, respectively. FIG. 2C displays a scatterplot correlating averaged decay-corrected gamma counts and fluorine content in blood samples (n=30 total); the Pearson correlation coefficient is r=0.9933.

FIGS. 3A-3G are results from visualization of acute footpad inflammation via in situ labeling of phagocytic immune cells with 89Zr labeled FERM nanoemulsion. FIG. 3A isplays representative PET/CT of mice 24 h after intravenous injection of (PFOB or PFCE) FERM nanoemulsion or control nanoemulsion without chelate (100 μCi, n=3 each), shown as % ID g-1. In FIG. 3B, ROI results of PET signal in paws are shown. FIG. 3C shows biodistribution of FERM nanoemulsion in excised tissues measured by γ-counting (decay-corrected) post-imaging. Composite 19F/1H MRI is shown in FIG. 3D from the mouse in FIG. 3A, where the scalebar is fluorine atoms per mL. FIG. 3E, shows quantification of fluorine content in footpads with and without carrageenan treatment. FIG. 3F exhibits the dose dependence of the measured % ID (100-1 μCi), as measured by ROI analysis of PET images; the inset shows the signal ratio of the right and left paws (R:L). No significant difference is observed in R:L between each dose (p>0.05). Acquisition times for PET and FMRI where 10 and 15 mins, respectively. Data are shown as mean±s.e.m. (n=3), where denotes P<0.05, ** is P<0.005 and *** is P<0.001 in unpaired t-tests.

FIGS. 4A-4G are results from multimodal PET-FMRI in IBD mice. FIG. 4A displays PET/CT images of three representative IBD mice 24 h after intravenous injection of FERM nanoemulsion (100 μCi); control (naïve) mice received the same FERM nanoemulsion dose. FIG. 4B shows composite FMRI/CT images of the mice in FIG. 4A, where the scalebar is fluorine atoms per mL. In FIG. 4C, superimposed FMRI and ¹H MRI slices in the mice from FIG. 4B is shown. Histograms (FIG. 4D, PET) and (FIG. 4E, FMRI) of probe quantification per voxel are shown for abdominal ROI encompassing entire peritoneum. FIGS. 4F and 4G are composite PET-FMRI slices (Mouse 2) showing coronal and transverse views, respectively, where FMRI=red, PET=blue, and overlap=magenta.

FIGS. 5A-5C are results from in vivo detection of TAMs and metastasis in 4T1 tumors using the nanoemulsions described herein. FIGS. 5A and 5B display representative PET/CT (left) and BLI images (right) of mice 2 and 5 weeks post-implantation with 4T1 breast cancer cells, respectively, where T=tumor and M=metastasis. BLI of metastasis in 5-week group (FIG. 5B, right) is acquired after shielding the primary tumor. FIG. 5C displays an overlay of ¹⁹F/¹H MRI slices in transverse and coronal views at tumor site from the same mouse as FIG. 5B and shows hotspots in tumor periphery consistent with TAM localization the metastasis is outside the image field of view. Images were acquired ˜24 h after injection with FERM nanoemulsion.

FIG. 6 is a scheme of a chemical synthesis of FHOA.

FIG. 7 is a scheme of a chemical synthesis of chelator 3, chelator 4, and FDFO.

FIGS. 8A and 8B are plots showing binding kinetics of ZrCl₄ to emulsions containing 10% FDK as a proportion of fluorocarbon. (A) is a lipid surfactant emulsion and absorbance is fit to a double exponential association model. (B) is a pluronic surfactant emulsion and absorbance fits to a single exponential association model.

FIG. 9 is a lead block with cutouts to support and hold tissue culture tubes during cell labeling with radioactive emulsion. To reduce radiation exposure and potential cell radiotoxicity during incubation periods (e.g., 1-24 hours) for ex-vivo cell labeling, cells and emulsions are placed in a tissue culture vessel and held by placing vessel in cutouts in a lead block, with 1 to 64 cutout wells to accommodate 1 to 64 cell culture vessels. The through-lead distance between the block cutouts is such that radiation expose, due to proximity to neighboring culture tubes that contain cells and radioemissive emulsion, is large enough to attenuate radioactive emission from neighboring vessels. To maintain cells at physiologic temperature (e.g., 37° C.), which promotes emulsion uptake in cells, the lead block is placed inside a tissue culture incubator. Alternative, a heating element is attached to the block with a temperature regulation feedback loop to maintain the block and vessels at physiological temperature. Other radiation absorbent metals such as Sn, Sb, W, Bi or other elements, alloys and condensed matter may serve as alternative block compositions.

FIG. 10 is a visualization of radiometal binding to emulsion by size exclusion chromatography and PET-CT.

FIG. 11 is an image showing in vivo cell tracking in mouse of splenocytes labeled ex vivo. Splenocytes were labeled by coincubation with FERM nanoemulsion for 4 hours, washed and then injected intravenously in mouse. Images shows PET-CT with FERM signal from labeled cells rendered in NIH-FIRE color-scale.

### DESCRIPTION

While the disclosed subject matter will be described in conjunction with the enumerated claims, it will be understood that the exemplified subject matter is not intended to limit the claims to the disclosed subject matter.

The disclosure generally provides molecular probe compositions and methods for in vivo nuclear imaging of inflammatory cells, such as macrophage, or alternatively, transferred cells that are used as part of a cell therapy. Nuclear imaging encompasses methods include positron emission tomography (PET) and single-photon emission computed tomography (SPECT). Optionally, fluorine-19 magnetic resonance imaging (FMRI) or fluorescence imaging are used for the imaging read-out. The compositions and methods described herein can also be a key component of a theragnostic medical procedure.

Inflammation is a defensive innate immune response toward invasive stimuli, featuring activation and recruitment of immune cells. Though it beneficially promotes pathogen clearance and tissue recovery, uncontrolled inflammatory responses drive disease pathobiology. In cancer, for instance, macrophages play a multifaceted role in disease progression and response to therapies. Tumor associated macrophages (TAMs) serve several pro-tumoral functions including the expression of growth, angiogenesis and lymphangiogenesis factors. Moreover, TAMs release matrix proteases leading to the suppression of adaptive responses. M1 macrophage subtype demonstrate high capacity to present antigens and activate polarized type I T-cell responses resulting in cytotoxicity towards tumor cells. Conversely, M2 macrophages have poor antigen presenting capacity, suppress inflammatory responses and Th1 adaptive immunity and thus contribute in hijacking the local immune system away from anti-tumor function. Tissue-resident macrophage number may be amplified through the recruitment and differentiation of circulating monocytes in the context of inflammation/tumor expansion. Many growth and differentiation factors like M-CSF, PGE2, IL-6 & IL-10 expressed in the tumor microenvironment have the potential to promote differentiation and polarization of recruited monocytes into TAMs of the M2 subtype. TAMs also exert immunosuppressive activity by releasing chemokines that preferentially attract T-cell subsets devoid of cytotoxic function and thus facilitate tumor progression and metastatic invasion. Therapeutically, TAMs are both effectors and modulators of immune responses to conventional cancer therapeutics, i.e., chemotherapy and radiotherapy. While radiotherapy directly induces double strand breaks and kills cancer cells, it is increasingly clear that tumor responses to radiotherapy are governed by immune responses. Moreover, focally delivered radiotherapy modulates systemic anti-tumor abscopal responses. Importantly, tumor irradiation has been reported to trigger polarization of macrophages from M2 toward M1 subtype resulting in T cell infiltration.

Within the last decade, therapeutics targeting immune checkpoints (i.e. CTLA-4, PD-1 and PD-L1) have revolutionized cancer therapy in subsets of cancer patients. There is tremendous interest in developing strategies to boost the efficacy of immune checkpoint inhibitors to broader patient populations. Interestingly, pre-clinical models have demonstrated that immune checkpoint inhibitors promote anti-tumor cytotoxic T lymphocyte responses to irradiation. Excitingly, in the PACIFIC trial of patients with locally advanced non-small cell lung cancer, the addition of durvalumab (anti PD-L1) after cytotoxic chemo-radiotherapy improved patient survival. More recently, it has been shown that tumors in mice treated with IPI-549, a selective PI3K gamma inhibitor that is currently in Phase II clinical testing for cancer, exhibited reduced myeloid cell accumulation, increased T cell recruitment and greater response to checkpoint inhibitors. IPI549 inhibits myeloid cell accumulation in tumors as well as suppress tumor growth from 50-100%, depending on the model. Alternatively, therapy induced inflammation within the tumor microenvironment drives cancer progression through TAMs. Thus, there is an urgent need for imaging probes and methods that can non-invasively monitor TAM burden in clinical trials to rationally optimize therapeutic strategies. Overall, oncology presents a key field of use for precise macrophage imaging, due to the diagnostic potential as well as the increasing focus on macrophages as therapeutic targets.

Diagnosis and treatment monitoring of autoimmune diseases is another example that could greatly benefit from precise macrophage detection in vivo. For example, inflammatory bowel disease (IBD), such as Crohn's disease and ulcerative colitis, is a non-curable autoimmune disease of gastrointestinal tract affecting millions of people worldwide. IBD is a premalignant condition that significantly increase the risk of colorectal cancer. The degree of risk of colorectal cancer depends on the anatomical extent, duration and age of onset of IBD. Resident macrophages in the colon play a key role in the homeostasis of the bowel, and macrophages derived from blood monocytes are important mediators of chronic inflammation in IBD along with Th1 and Th2 type T cells. Computed tomography (CT) and MRI are increasingly being used for evaluation of IBD, and the diagnosis is commonly confirmed by barium-enhanced x-ray scans and ‘gold-standard’ colonoscopic biopsy. Colonoscopic biopsy is invasive and requires multiple tissue bites for diagnosis, which may result in sampling errors and cause patient discomfort, thus driving the need for more precise diagnostics for staging and monitoring treatment course. Improved non-invasive biomarkers for IBD are highly sought after, both to improve the precision of the increasing volume of clinical trials through enhancing stratification and secondary end points, and for improved diagnosis and patient care.

Overall, there are a multitude of other serious human diseases and conditions that could benefit from precise macrophage inflammation imaging diagnostics, including, but not limited to appendicitis; cardiovascular inflammatory conditions, including for example, myocarditis, atherosclerosis, myocardial infarction; diticulitis; bacterial infection; viral infection, for example, COVID-19 coronavirus infection; multiple sclerosis; organ and tissue transplant rejection; metabolic diseases (e.g., NASH); graft-versus host disease; ischemia; and other autoimmune and infectious diseases.

Other autoimmune diseases include, for example, acute disseminated encephalomyelitis (ADEM), Addison's disease, antiphospholipid antibody syndrome (APS), aplastic anemia, autoimmune hepatitis, autoimmune skin disease, coeliac disease, Crohn's disease, Diabetes mellitus (type 1), Goodpasture's syndrome, Graves' disease, Guillain-Barre syndrome (GBS), Hashimoto's disease, lupus erythematosus, multiple sclerosis, myasthenia gravis, opsoclonus myoclonus syndrome (OMS), optic neuritis, Ord's thyroiditis, oemphigus, polyarthritis, primary biliary cirrhosis, psoriasis, rheumatoid arthritis, Reiter's syndrome, Takayasu's arteritis, temporal arteritis, warm autoimmune hemolytic anemia, Wegener's granulomatosis, alopecia universalis (e.g., inflammatory alopecia), Chagas disease, chronic fatigue syndrome, dysautonomia, endometriosis, hidradenitis suppurativa, interstitial cystitis, neuromyotonia, sarcoidosis, scleroderma, ulcerative colitis, vitiligo, and vulvodynia.

Other infectious diseases include, for example, polymyositis, dermatomyositis, spondyloarthropathies such as ankylosing spondylitis, anti-phospholipid syndrome, and polymyocysitis.

In all of these human conditions, inflammation is a key component to the pathobiology. The occurrence of inflammation hotspots in the body is accompanied by the presence of inflammatory infiltrates, particularly endogenous macrophage, T and B cells, neutrophils, natural killer (NK) cells, and dendritic cells (DCs). The ability to image inflammatory infiltrates with high cell specificity in situ has profound diagnostic utility for precise early detection, stratification and treatment monitoring.

Conventional tissue contrast-based inflammation imaging, such as via ¹H MRI and computerized tomography (CT), detects non-cell-specific anatomical lesions at late stage. Thus, there is keen interest in developing molecular imaging probes that target inflammation markers and inflammatory cells to achieve increased specificity, earlier diagnosis, and improved therapeutic outcomes.

The PET probe fluorodeoxyglucose (FDG), a glucose analog labeled with 18-fluorine (t_{1/2}=108 min), serves as an imaging biomarker for numerous inflammatory diseases. While increased FDG uptake is observed in macrophages and inflammatory lesions, normally high physiologic uptake of FDG in certain tissues can obscure the evaluation of the specific inflammatory component of diseases such as in cancer, IBD, glomerular nephritis, and an array of cardiovascular inflammatory disorders. For example, FDG tumor scans are unable to distinguish between TAMS and dividing tumor cells, as both are metabolically active and take up the agent. In the case of IBD, physiologic bowel uptake of FDG can be quite intense, especially in patients on Metformin.

To improve inflammation specificity, macrophage uptake of ¹⁸F-mannose has been used for PET of atherosclerotic plaque inflammation. Other small molecule radiotracers targeting inflammatory markers, such as cytokines, translocator proteins, enzymes, and integrin receptors, have been designed for enhanced specificity with varying degrees of success. Other notable approaches use nanoparticle PET tracers for macrophages, include polyglucose labeled with ¹⁸F or ⁶⁴Cu, and dextran labeled with ⁸⁹Zr-DFO, with their use demonstrated preclinically for cardiovascular and TAM imaging.

Another use of the methods and compositions described herein is in the field of cell therapy, where live cells, either autologous or allogenic, prepared in the laboratory, are transferred to the patient to achieve a therapeutic outcome. Numerous clinical trials are underway using cell-based therapeutic strategies to treat individuals with genetic and neurological disorders, as well as chronic conditions such as cancer and autoimmunity. Emerging cellular therapeutic strategies have the potential to provide patient-specific, less toxic and more efficacious treatments through the repair or activation of endogenous functions. Currently, more than 10,000 clinical trials worldwide are underway involving the transplantation of cells (https://clinicaltrials.gov). Importantly, immunotherapy using live cells is emerging as the fourth pillar in the treatment of cancer, along with surgery, chemotherapy and radiation. The use of immunotherapeutic lymphocyte cell products, particularly engineered T cells, has made transformative inroads towards ‘curative’ clinical outcomes for certain cancers. Additionally, stem cell technologies have allowed scientists to consider the use of cellular therapy as an option to treat many degenerative disorders and potentially regenerate tissues. Conditions such as neurodegenerative diseases, myocardial infarction, and spinal cord injuries are prime targets for regenerative cell therapy. Examples of contemporary cell therapy include, but are not limited to chimeric antigen receptor (CAR) T cells to treat blood cancers and solid tumors; genetically modified T cells to treat autoimmune and infectious diseases; NK cells to treat cancers; mesenchymal stem cells (MSCs) to treat inflammation; and umbilical cord blood stem cells to treat various conditions; CD34+ stem cells for various conditions including cancers, in the central nervous system (CNS).

A common need for developers of cell therapies is a non-invasive means to visualize the fate of cells in vivo following injection. Imaging cell trafficking can provide crucial feedback regarding the biodistribution, persistence, optimal routes of delivery and therapeutic doses. On the regulatory side, emerging new therapies, such as those using immunotherapeutic cells and stem cells, can be slow to gain regulatory approvals partly because clinical researchers are unable to verify where the cells go immediately after patient inoculation, as well as their fate days and weeks later.

The disclosure provides, among other things, a class of molecules, fluorocarbons, that are formulated according to the instant disclosure, with aid of a surfactant into stable, nontoxic nanoemulsions which are then functionalized for nuclear imaging by methods described herein. The biological inertness and high oxygen solubility of fluorocarbons have made them candidates for oxygen-carrying blood substitutes since the 1980s. In the clinic, microbubbles made from fluorocarbons (e.g., Definity, Imagent) are routinely used for contrast-enhanced ultrasound imaging. There are no known enzymes that degrade fluorocarbons in vivo, and they do not degrade at typical lysosomal pH values. Perfluorocarbons have simultaneous lipophobic and hydrophobic properties and do not incorporate into cell membranes. Clearance of fluorocarbon agents from the body occurs via the reticuloendothelial system (RES) followed by lung exhalation. Historically, various fluorocarbon molecules have been evaluated for clinical use as artificial oxygen carriers in large doses (˜10 g/kg). The fluorocarbon emulsions have been tested for biological safety, with few observed adverse effects to viability or function in cells. Numerous studies have investigated the impact of fluorocarbon cell labeling in primary immune cells using a variety of sensitive in vitro assays, for example in the context of murine DCs, T cells, and stem cells, and MSCs. In vitro studies involve ex vivo fluorocarbon labeled primary human DCs (48); cells that were assayed for viability, maturation phenotype, cytokine production, T cell stimulatory capacity, and chemotaxis, and no difference in these parameters was observed between labeled and unlabeled cells.

In the past decade, fluorocarbon emulsions featuring a small size (100-200 nm in diameter, e.g., nanoemulsion) have emerged as useful agents for imaging. As an example, given the high endocytic activity of monocytes and macrophages and their heavy involvement in inflammatory processes, nanometer-sized emulsion droplets mimicking ‘pathogens’ are a powerful cell delivery approach following intravenously delivery, exploiting highly-evolved cell functions for efficient intracellular probe labeling in situ, to detect localized sites of inflammation. The emulsion droplets coalesce into phagocyte lysosomal vesicles and macropinosomes, thus escaping osmatic pressure forcing cell efflux and yields durable labeling, in contrast to small molecule tracers. Methods have used intravenously administered fluorocarbon nanoemulsions to enable background-free ‘hotspot’ fluorine-19 MRI (FMRI) detection. The FMRI hot-spots can be quantified and the signal is linearly proportional to the macrophage burden. The nanoemulsion droplets are scavenged in situ by cells of the reticuloendothelial system (RES), particularly monocytes, macrophages, but also neutrophils and DCs. In situ macrophage labeling with fluorocarbon nanoemulsion, detected by FMRI, has been used in a multitude of preclinical inflammation models, with macrophage specificity validated histologically.

As another example of fluorocarbon nanoemulsion imaging agents, cells are labeled in culture using a fluorocarbon nanoemulsion formulation. Following transfer to the subject, the labeled cells are detected in vivo using FMRI. The fluorine inside the cells yields positive-signal ‘hot-spot’ images, with no background signal due to the paucity of detectable fluorine atoms in host tissues. Images can be quantified to measure apparent cell numbers at sites of accumulation, thereby enabling ‘in vivo cytometry’. The sensitivity limits of detection are on the order of 104-105 cells/voxel with in vivo cytometry. In vivo cytometry has been used to visualize DCs in mouse and human cancer patients, T cells in a mouse models, NK cells, neuronal stem cells and mesenchymal stem cells. Overall, a major limitation of FMRI detection of fluorocarbon labeled cells in vivo using either in situ phagocyte labeling or in vivo cytometry is that capabilities for non-proton MRI (i.e., FMRI) is uncommon in a clinical setting.

Fluorocarbon emulsions have also been functionalized for fluorescence imaging by conjugation of dyes to fluorocarbon molecules or to lipids on the surface. While surface conjugation through hydrophobic interaction is widely used, the affinity of dye is relatively weak, and dissociation and bleaching has been observed in vivo. Heavily fluorinated fluorophores that reside in the fluorocarbon oil have also been reported.

The instant disclosure therefore provides, among other things, compositions and methods to produce a compound comprising fluorocarbon and radioisotope for medical uses, particularly for medical imaging and drug delivery. Production and use of such emulsions contain process elements that include: (i) chemical synthesis of fluorous metal chelates, (ii) formulation of stable emulsions with chelates solubilized in the fluorous phase, (iii) radiolabeling of pre-formed emulsions, (iv) purification of radiolabeled emulsions and/or buffering emulsions for in vivo use, (v) delivery to medical scan subjects, (vi) imaging readouts using PET, SPECT, FMRI, ¹H MRI and/or CT, and (vii) computer-based diagnostic readouts to anatomically localize and quantify emulsion hotspots of accumulation in vivo. Optional process elements can also include: (a) ex vivo labeling of cell therapy products or other isolated cells of interest with radiolabeled emulsion, often achieved by co-incubation of radiolabeled emulsion with cells in culture under physiologic conditions for various time periods in order to combine the cells and emulsion, (b) one or more wash steps to removed uncombined radiolabeled emulsion, (c) assay methods to quantify radioactive incorporation into cells of interest, and (d) delivery of combined cell product to patient receiving therapy. Optionally, the radiolabeled emulsions described herein can be delivered to the patient and serves as a therapeutic agent, for example as a radiotherapy to help eradicate solid tumors or disseminated disease.

In one embodiment of the invention, functionalized fluorocarbon emulsions are employed to incorporate a fluorous hydroxamic acid chelator that captures radioisotopes, such as ⁸⁹Zr, ⁶⁴Cu, ⁶⁸Ga, into the fluorous phase of preformed emulsion via a premix step, with a high degree of agent stability in vivo. Following, e.g., intravenous delivery, the emulsion is taken up by phagocytic macrophages. The usefulness of the radioactive emulsion is demonstrated in the imaging of macrophage-associated inflammatory and tumoral disease models by both PET and FMRI techniques. Good correlation is observed between signals produced from PET imaging and FMRI imaging in these applications. The high sensitivity of PET imaging enables unambiguous whole-body imaging of macrophage burden using radiolabeled emulsion at clinically relevant dosages and scan times. The present invention provides compositions of such compound as well as methods to produce and image said probe compounds for diagnostic and therapeutic uses.

The disclosure provides a composition comprising (i) at least one fluorocarbon and (ii) at least one fluorous hydroxamic acid chelator, wherein the at least one fluorous hydroxamic acid chelator is at least partially soluble in the at least one fluorocarbon. The composition comprising (i) the at least one fluorocarbon and (ii) the at least one fluorous hydroxamic acid chelator can be formulated into an emulsion, where the emulsion forms, e.g., a colloidal suspension comprising droplets having a diameter of less than about 200 nm, less than about 150 nm, less than about 100 nm, from about 5 nm to about 500 nm, such as form about 5 nm to about 100 nm, about 5 nm to about 200 nm, about 50 nm to about 200 nm, about 50 nm to about 150 nm, about 25 nm to about 250 nm, about 100 nm to about 350 nm, about 5 nm to about 50 nm, about 250 nm to about 450 nm, about 100 nm to about 200 nm, or about 20 nm to about 120 nm as determined by dynamic light scattering (DLS). It can be advantageous to have droplets having a diameter of less than about 200 nm because, among other things, such droplets can be subjected to sterile filtering (e.g., they will pass through a sterile filter). Alternatively, or in addition, it can be advantageous to have droplets having a diameter of less than about 200 nm because such droplets can be subjected to a washing step where, e.g., excess chelator can be substantially removed. Alternatively, or in addition, it can be advantageous to have droplets having a diameter of less than about 200 nm because such droplets will not be captured along with, e.g., cellular material (e.g., cells) when compositions comprising (i) at least one fluorocarbon and (ii) at least one fluorous hydroxamic acid chelator are subjected to centrifugation in the presences of, e.g., cellular material.

As used herein, the term “at least partially soluble” refers to, for example, the at least one fluorous hydroxamic acid chelator having a solubility of from about 1 mM to 5 mM in the at least one fluorocarbon.

The at least one fluorocarbon can be selected from the group consisting of perfluorooctyl bromide (PFOB), perfluoro-15-crown-5-ether (PFCE), perfluoropolyethers (PFPE), perfluorotrialkylamine (PFTA), perfluorodecalin (PFD), perfluorohexane (PFH), perfluorononane (PFN), hexafluorobenzene (HFB), PERFECTA, perfluoro-tert-butyl-cyclohexane (PFTBC), and the like, each having the formulae:

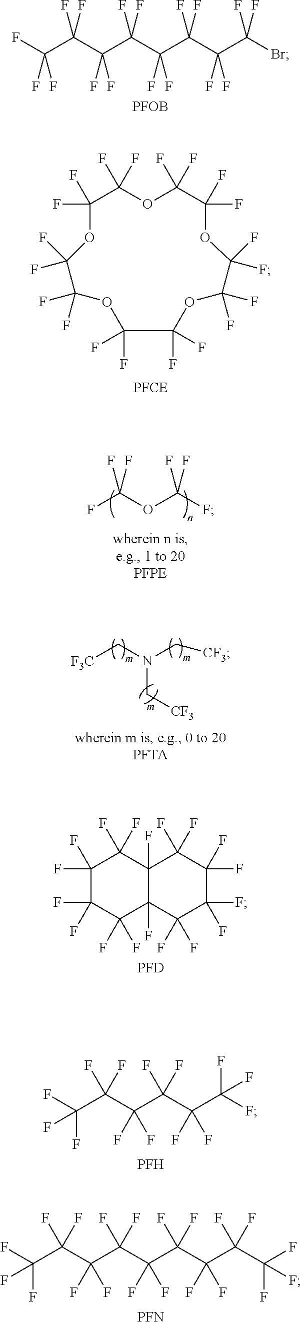

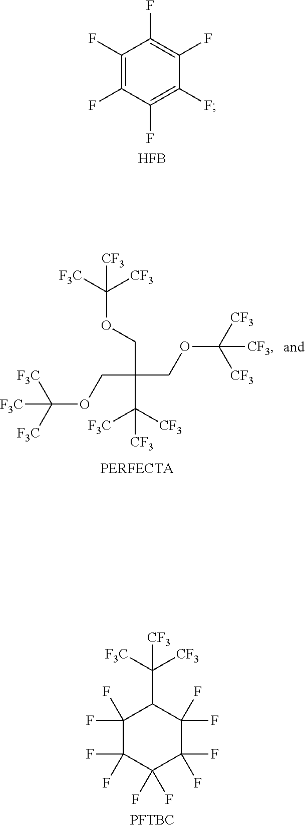

respectively.

Examples described herein include those in which one fluorocarbon is used in the compositions described herein comprising (i) at least one fluorocarbon and (ii) at least one fluorous hydroxamic acid chelator. But instances are contemplated where two or more fluorocarbons can be used (e.g., two, three, four, five or six fluorocarbons).

The at least one fluorous hydroxamic acid chelator used in the compositions described herein comprising (i) at least one fluorocarbon and (ii) at least one fluorous hydroxamic acid chelator can be selected from the group consisting of:

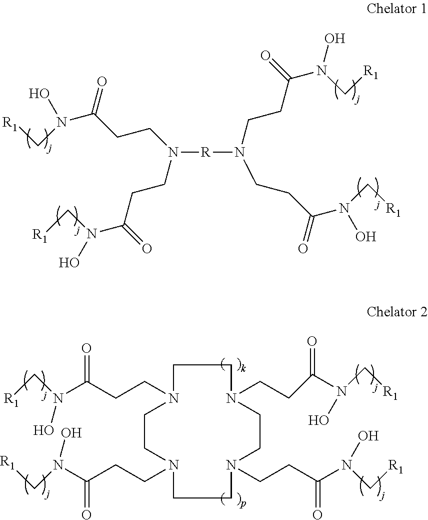

wherein R is a chemical residual, such as a linker, each j is independently 1 to 20; k and p are each independently 0 to 3; each R₁ is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein each p and q is independently 0 to 20;

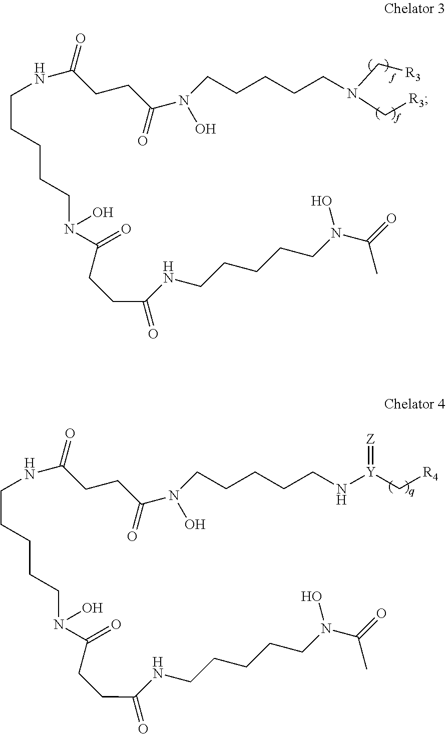

wherein each f and q is independently from 0 to 20 (e.g., 2 to 4); Y and Z are each selected from the group consisting of C, N, O, Si, P, and S (e.g., Y can be C, S or P; and Z can be O, NZ¹S, wherein Z¹ can be H or alkyl; or Y═N can form the groups C═O, C═S, C═N—OH, P═O, S═O); each R₃ and R₄ is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein p and q are 0 to 20 (e.g., 1 to 20 and 3 to 8); and

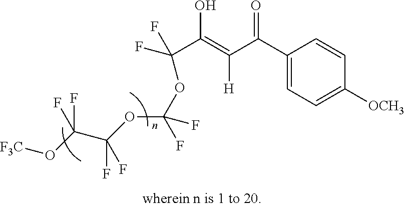

As used herein, the term linker includes any moiety R that can link each side of, e.g., Chalator 1. Examples of suitable linkers include aliphatic linkers (e.g., alkyl linkers, including C₃-C₆-alkyl linkers, and cycloalkyl linkers, including C₃-C₆-cycloalkyl linkers), aromatic linkers (e.g., aryl linkers, including C₈-C₁₀-aryl linkers), (poly)alkylene glycol linkers (e.g., -(alkyl-O)_{q}— linkers, wherein q=an integer from 1 to 1000, e.g., from 1 to 500, 1 to 50, and 1 to 5), and the like and combinations thereof.

The term “alkyl” as used herein refers to substituted or unsubstituted straight chain and branched alkyl groups and cycloalkyl groups having from 1 to 40 carbon atoms (C₁-C₄₀), 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. Representative substituted alkyl groups can be substituted one or more times with any of the groups listed herein, for example, amino, hydroxy, cyano, carboxy, nitro, thio, alkoxy, and halogen groups. The term “alkyl” also encompasses substituted or unsubstituted straight chain and branched divalent alkyl groups, such as —CH₂—, —CH₂CH₂—, —CH₂CH₂CH₂—, and —CH₂CH(CH₃)CH₂—.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “substituted” as used herein refers to a group (e.g., alkyl and aryl) or molecule in which one or more hydrogen atoms contained thereon are replaced by one or more substituents. The term “substituent” as used herein refers to a group that can be or is substituted onto a molecule or onto a group. Examples of substituents include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R, wherein R can be, for example, hydrogen, alkyl, -(alkyl-O)_{q}— (wherein q=an integer from 1 to 1000, e.g., from 1 to 500, 1 to 50, and 1 to 5), acyl, cycloalkyl, aryl, aralkyl, heterocyclyl, heteroaryl, or heteroarylalkyl, wherein each alkyl, acyl, cycloalkyl, aryl, aralkyl, heterocyclyl, heteroaryl, or heteroarylalkyl can each be substituted. A non-limiting example of an -(alkyl-O)_{q}— group includes groups of the formula —CH₂CH₂OCH₂CH₂OCH₂CH₂O— and the group of the formula —CH₂CH₂OCH₂CH₂O—, each of which can generally be referred to as (poly)ethylene glycol (PEG) linkers.

The disclosure provides, for example, compositions comprising a fluorous hydroxamic acid chelator, wherein the chelator is selected from the group consisting of:

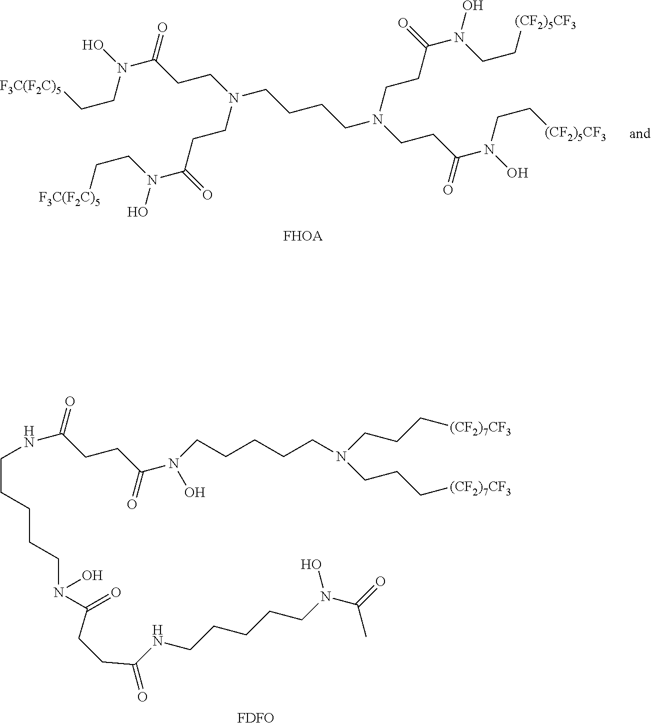

The at least one fluorous hydroxamic acid chelator can be soluble in fluorocarbons such as the ones described herein. Heating, stirring or sonication can be applied to dissolve the fluorous hydroxamic acid chelator in the fluorocarbons. Such compositions can comprise the at least one fluorous hydroxamic acid chelator dissolved in fluorocarbon at a molar concentration of 0 to 0.1 mol/L.

Successful encapsulation of radioisotope in fluorocarbon oil (e.g., at least one fluorocarbon), which is both highly hydrophobic and lipophobic, can include the use of a potent fluorous chelator (e.g., at least one fluorous hydroxamic acid chelator) preloaded in fluorocarbon oil. The fluorous chelator extracts radioisotopes from the aqueous solution to the fluorocarbon oil. For a chelator to be soluble in fluorocarbon, the chelator may need to have a significant amount of fluorine atoms. Empirically, mass of fluorine atoms in a molecule needs to be greater than 50% of its molecular weight in order to dissolve in common fluorocarbons at sufficient concentration. While there are many chelators available for binding of radioisotopes or radiometals, there are rarely any chelators that are soluble in fluorocarbon due to a lack of fluorine atoms.

Some fluorous chelators, in particular fluorous β-diketones, were found to be useful in the extraction of metals in supercritical carbon dioxide (e.g., U.S. Pat. Nos. 5,730,874; and 5,965,025). Several other kinds of fluorous chelators that bind with iron and other paramagnetic metals are also known. These chelators can extract some paramagnetic metals (e.g. Fe³⁺, Gd³⁺, and Mn²⁺) from an aqueous phase into the fluorocarbon oil. They were used to boost the sensitivity of fluorine magnetic resonance imaging (FMRI) by enhancing the relaxation rates of fluorine atoms. But the availability of a fluorous chelator is still very limited, especially their use in biomedical areas like the ones described herein.

The present disclosure provides compositions comprising fluorous hydroxamic acids that can be used to bind with radioisotopes. Hydroxamic acid is a class of compound bearing one or more of the following functional group:

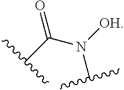 [SMILES: *C(=O)N(*)O]

Hydroxamic acids are known as potent chelators for a wide range of metal in coordination chemistry. While compounds bearing one hydroxamic unit can bind with metals, those bearing multiple hydroxamic units generally have stronger binding efficiency, due to the chelation effect. In nature, bacteria produce various kind of hydroxamic acids, such as siderophores, to participate in iron assimilation and metabolism. In the clinic, products of hydroxamic acids (e.g., Desferal) have been used to treat iron overdose or aluminum toxicity. In industry, derivatives of hydroxamic acid are used for the selective extraction of rare earth metals from raw materials. However, preparation and utility of fluorous hydroxamic acids has not been widely reported. For example, several fluorous mono-hydroxamic acids were prepared for extraction of iron with supercritical CO₂. See, e.g., J. Chromatogr. A 1997, 770:85-91. In one example, fluorous polymers containing hydroxamic acid units were used for sequestration of trace metals in water. See, e.g., Angew. Chem. Int. Ed. 2000, 39: 1039-1042. In another example, a series of fluorous mono- or multi-hydroxamic acids were prepared using Aza-Michael reaction between a fluorous acrylamide and primary or secondary amines. See, e.g., Synth. Commun. 2002, 32: 3779-3790. However, none of the foregoing explored the use of fluorous hydroxamic acid in biomedical areas, such as in cells, animals or humans, possibly due to their highly hydrophobic nature. The Aza-Michael reaction has been employed herein to prepare some of the fluorous multi-hydroxamic acids described herein.

The disclosure provides compositions comprising (i) at least one fluorocarbon and (ii) at least one fluorous hydroxamic acid chelator used to capture metals ions (e.g., metal ions chelated to the at least one fluorous hydroxamic acid chelator), wherein the metal ions are selected from the group consisting of Zr⁴⁺, Th⁴⁺, Tc⁴⁺, Pb⁴⁺, Po⁴⁺, Ti⁴⁺, Sn⁴⁺, Mn⁴⁺, Al³⁺, Bi³⁺, Co³⁺, Ga³⁺, Au³⁺, Fe³⁺, Sc³⁺, Ti³⁺, Eu³⁺, Gd³⁺, Ho³⁺, Sm³⁺, Lu³⁺, Er³⁺, Pr³⁺, Yb³⁺, Tm³⁺, Dy³⁺, Nd³⁺, Ce³⁺, Tb³⁺, Y³⁺, Cr³⁺, Mn³⁺, In³⁺, Be²⁺, Co²⁺, Cu²⁺, Ga²⁺, Fe²⁺, Pb²⁺, Mg²⁺, Hg²⁺, Po²⁺, Ra²⁺, Ti²⁺, UO2²⁺, Yb²⁺, Zn²⁺, Mn²⁺ and Ni²⁺.

The disclosure provides compositions comprising (i) at least one fluorocarbon and (ii) at least one fluorous hydroxamic acid chelator used to capture radioisotopes (e.g., radioisotopes are chelated to the at least one fluorous hydroxamic acid chelator), wherein the radioisotopes are selected from the group consisting of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺ and ⁸⁶Y³⁺.

The disclosure provides compositions comprising (i) at least one fluorocarbon, (ii) at least one fluorous hydroxamic acid chelator, and (iii) at least one radioisotope, wherein the radioisotopes are selected from the group consisting of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺, and ⁸⁶Y³⁺.

The disclosure provides compositions comprising (i) at least one fluorocarbon and (ii) at least one fluorous hydroxamic acid chelator used to capture (e.g., chelate) ⁸⁹Zr.

The disclosure provides compositions comprising (i) at least one fluorocarbon, (ii) at least one fluorous hydroxamic acid chelator, and (iii) ⁸⁹Zr.

The compound (e.g., at least one fluorous hydroxamic acid chelator complexing ⁸⁹Zr) can be purified to remove unbound radioisotopes before use. Purification methods include size-exclusion gel filtration, centrifugation, ultra-centrifugation, dialysis, high-throughput size-exclusion chromatography. But the compound can be used without purification.

The compositions comprising (i) at least one fluorocarbon and (ii) at least one fluorous hydroxamic acid chelator can be formulated into an emulsion.

A column can be used and imaged using PET/CT to identify fractions of radioisotope bound to fluorocarbon and free radioisotope.

The compositions comprising (i) at least one fluorocarbon, (ii) at least one fluorous hydroxamic acid chelator, and (iii) at least one radioisotope can be administered to a subject (e.g, animal or human) for non-invasive imaging. For example, the compositions comprising (i) at least one fluorocarbon, (ii) at least one fluorous hydroxamic acid chelator, and (iii) ⁸⁹Zr can be administered to a subject (e.g, animal or human) for non-invasive imaging. As used herein, the term “administered” includes, without limitation, orally, parenterally, by inhalation spray, topically, by eyedrops, rectally, nasally, buccally, vaginally or via an implanted reservoir, wherein the term “parentally”, as used herein, comprises subcutaneous, intracutaneous, intravenous, intramuscular, intra-articular, intrasynovial, instrasternal, intrathecal, intralesional and intracranial injection or infusion techniques.

The compositions comprising (i) at least one fluorocarbon, (ii) at least one fluorous hydroxamic acid chelator, and (iii) at least one radioisotope can be used to ex vivo label cells, cell products or cell therapies which are destined for subjects (e.g., animals or humans). For example, compositions comprising (i) at least one fluorocarbon, (ii) at least one fluorous hydroxamic acid chelator, and (iii) ⁸⁹Zr can be used to label cells, cell products or cell therapies.

The disclosure also provides a non-invasive imaging method comprising:
(a) administering to a subject a composition comprising The compositions comprising (i) at least one fluorocarbon, (ii) at least one fluorous hydroxamic acid chelator, and (iii) at least one radioisotope, wherein at least one fluorocarbon is selected from the group consisting of PFOB, PFCE, PFPE, PFTBC, and PFTA; the at least one radioisotope is selected from the group consisting of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺ and ⁸⁶Y³⁺; and the at least one fluorous hydroxamic acid chelator is selected from the group consisting of:

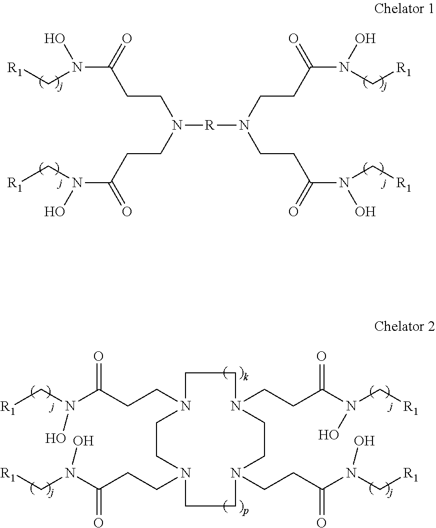

wherein R is a chemical residual, such as a linker, each j is independently 1 to 20; k and p are each independently 0 to 3; each R, is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein each p and q is independently 0 to 20;

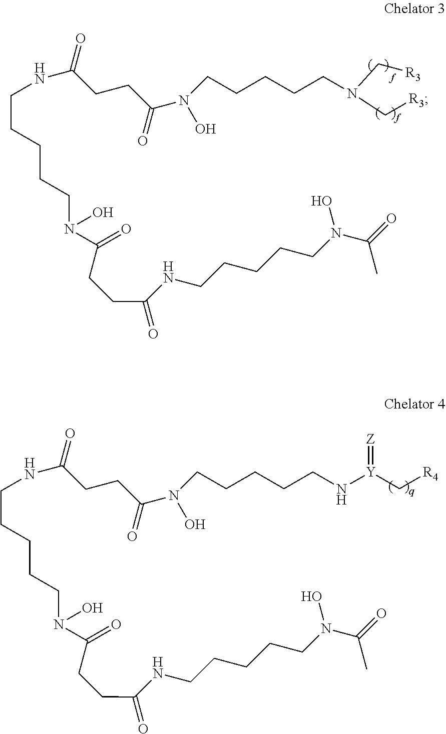

wherein each f and q is independently from 0 to 20 (e.g., 2 to 4); Y and Z are each selected from the group consisting of C, N, O, Si, P, and S (e.g., Y can be C, S or P; and Z can be O, NZ¹S, wherein Z¹ can be H or alkyl; or Y═N can form the groups C═O, C═S, C═N—OH, P═O, S═O); each R₃ and R₄ is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein p and q are 0 to 20 (e.g., 1 to 20 and 3 to 8); and

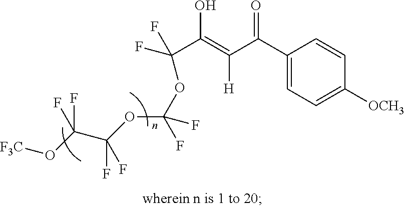

and
(b) detecting distribution of the said compound in the subject using at least one imaging modality, wherein the imaging modality is selected from the group consisting of positron emission tomography imaging (PET), magnetic resonance imaging (MRI), fluorine-19 magnetic resonance imaging (FMRI), computed tomography (CT), single-photon emission computed tomography (SPECT), fluorescence imaging, and luminescent imaging.

The disclosure also provides a cell-tracking method comprising:
(a) administering to a subject one or more cells comprising a composition comprising (i) at least one fluorocarbon, (ii) at least one fluorous hydroxamic acid chelator, and (iii) at least one radioisotope, wherein the at least one fluorocarbon is selected from the group consisting of PFOB, PFCE, PFPE, PFTBC, and PFTA; the at least one radioisotope is selected from the group consisting of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺ and ⁸⁶Y³⁺; the at least one fluorous hydroxamic acid chelator is selected from the group consisting of:

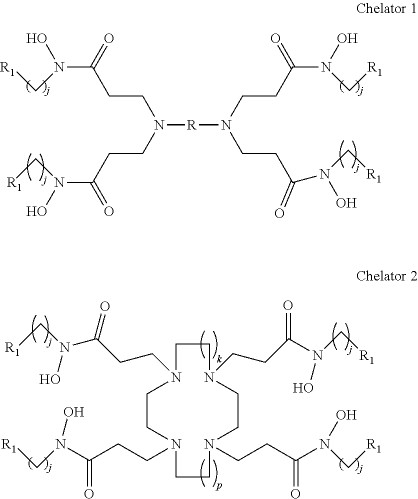

wherein R is a chemical residual, such as a linker; each j is independently 1 to 20; k and p are each independently 0 to 3; each R₁ is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein each p and q is independently 0 to 20;

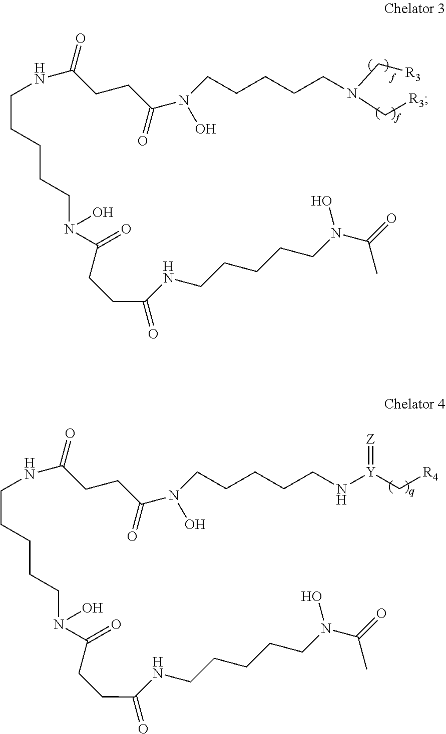

wherein each f and q is independently from 0 to 20 (e.g., 2 to 4); Y and Z are each selected from the group consisting of C, N, O, Si, P, and S (e.g., Y can be C, S or P; and Z can be O, NZ¹S, wherein Z¹ can be H or alkyl; or Y═N can form the groups C═O, C═S, C═N—OH, P═O, S═O); each R₃ and R₄ is independently selected from the group consisting of —(CF₂)ₚ—CF₃, —O—(CF₂)ₚ—CF₃, —O—CF₂—(OCF₂CF₂)ₚ—OCFₐ and —[(CH₂)_{q}(CF₂)ₚCF₃]₂, wherein p and q are 0 to 20 (e.g., 1 to 20 and 3 to 8); and

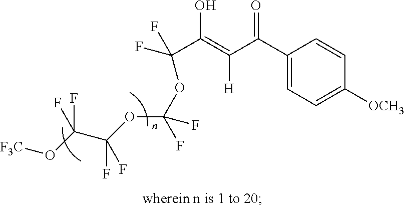

and
(b) detecting the location of cells in the subject using at least one imaging modality, wherein the imaging modality is selected from the group consisting of PET, ¹H MRI, FMRI, CT, SPECT, fluorescence imaging and luminescent imaging.

The composition comprising (i) at least one fluorocarbon, (ii) at least one fluorous hydroxamic acid chelator, and (iii) at least one radioisotope can be used for PET imaging. For example, a composition comprising (i) at least one fluorocarbon, (ii) at least one fluorous hydroxamic acid chelator, and (iii) ⁸⁹Zr can be used for PET imaging.

Alternatively, or in addition to PET imaging, the composition comprising (i) at least one fluorocarbon, (ii) at least one fluorous hydroxamic acid chelator, and (iii) at least one radioisotope can be used for FMRI. For example, the composition comprising (i) at least one fluorocarbon, (ii) at least one fluorous hydroxamic acid chelator, and (iii) ⁸⁹Zr can be used for FMRI. The compound comprising (i) fluorocarbon, (ii) fluorous hydroxamic acid chelator, and (iii) at least one radioisotope can therefore be used for simultaneous PET and FMRI using one or more imaging equipment. For example, a composition comprising (i) at least one fluorocarbon, (ii) at least one fluorous hydroxamic acid chelator, and (iii) ⁸⁹Zr can be used for PET and FMRI using one or more equipment.

The disclosure also provides a diagnostic composition comprising (i) at least one fluorocarbon, (ii) at least one fluorous hydroxamic acid chelator, and (iii) at least one radioisotope. For example, the disclosure also provides a diagnostic composition comprising (i) at least one fluorocarbon, (ii) at least one fluorous hydroxamic acid chelator, and (iii) at least one radioisotope for the diagnosis of inflammatory diseases or tumoral diseases.

The disclosure also provides a therapeutic composition comprising (i) at least one fluorocarbon, (ii) at least one fluorous hydroxamic acid chelator, and (iii) at least one radioisotope.

The disclosure also provides a therapeutic composition comprising (i) at least one fluorocarbon, (ii) at least one fluorous hydroxamic acid chelator, and (iii) at least one radioisotope for the treatment of inflammatory or tumoral diseases.

As stated herein, the disclosure provides compositions and methods for medical imaging, in particular positron emission tomography (PET), for non-invasive diagnosis of diseases associated with macrophage infiltration, such as inflammatory diseases and tumoral diseases. Subjects (e.g., cells, animals, and humans) treated with the compositions described herein can be visualized using PET or MRI or both. The disclosure also provides methods for the preparation, radiolabeling, and use of such compositions comprising (i) at least one fluorocarbon, (ii) at least one fluorous hydroxamic acid chelator, and (iii) at least one radioisotope for non-invasive imaging. Steps for preparing such compositions comprising (i) at least one fluorocarbon, (ii) at least one fluorous hydroxamic acid chelator, and (iii) at least one radioisotope can include one or more of the following:
(I) preparing the at least one fluorous hydroxamic acid chelator selected from the group consisting of

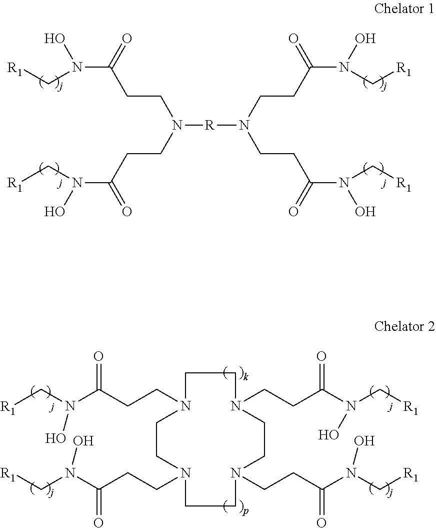

wherein R is a chemical residual, such as a linker; each j is independently 1 to 20; k and p are each independently 0 to 3; each R₁ is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein each p and q is independently 0 to 20;

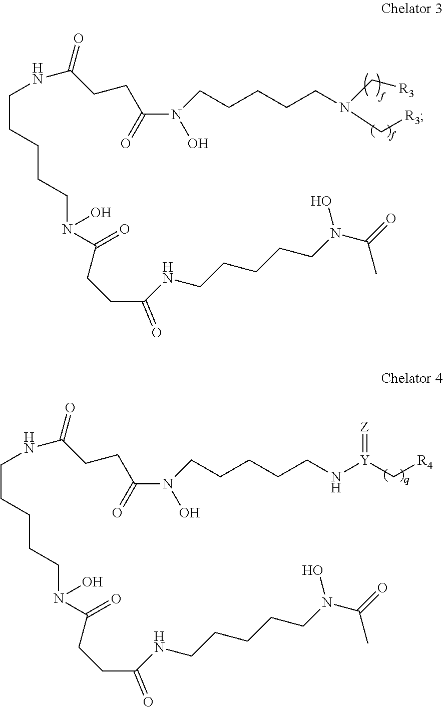

wherein each f and q is independently from 0 to 20 (e.g., 2 to 4); Y and Z are each selected from the group consisting of C, N, O, Si, P, and S (e.g., Y can be C, S or P; and Z can be O, NZ¹S, wherein Z¹ can be H or alkyl; or Y═N can form the groups C═O, C═S, C═N—OH, P═O, S═O); each R₃ and R₄ is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein p and q are 0 to 20 (e.g., 1 to 20 and 3 to 8); and

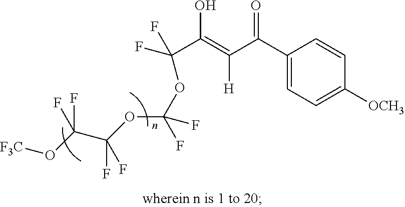

(II) contacting (e.g., dissolving or partially dissolving) the at least one fluorous hydroxamic acid chelator with the at least one fluorocarbon, wherein the fluorocarbons are selected from the group consisting of perfluorooctyl bromide (PFOB), perfluoro-15-crown-5-ether (PFCE), perfluoropolyethers (PFPE), perfluorotrialkylamine (PFTA), perfluorodecalin (PFD), perfluorohexane (PFH), perfluorononane (PFN), hexafluorobenzene (HFB), PERFECTA and others (e.g., perfluoro-tert-butyl-cyclohexane (PFTBC));
(III) emulsifying the (i) at least one fluorocarbon and (ii) at least one fluorous hydroxamic acid chelator with at least one surfactant to obtain an emulsified composition, such as by using high-pressure homogenization, extrusion, rotor-stator homogenization, ultrasonication, high-speed blending, membrane emulsification, microchannel emulsification, vortex, stirring, membrane filtration, pressure filtration;
(IV) radiolabeling the emulsified composition with at least one radioisotope to give a radiolabeled composition, wherein the radioisotopes are selected from the group consisting of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺ and ⁸⁶Y³⁺;
(V) optionally purifying the radiolabeled composition to give a purified composition using techniques, such as size-exclusion chromatography, centrifugation, ultrafiltration, dialysis and high-throughput size-exclusion chromatography;
(VI) formulating purified composition in a buffer suitable for parenteral administration, such as through size-exclusion chromatography or addition of components to the emulsion;
(VII) administering the radiolabeled composition or the purified composition to subjects (e.g., cells, animals, humans); and
(VIII) visualizing the administered composition in the subject with imaging techniques, such as PET, ¹H MRI, FMRI, CT, SPECT, fluorescence imaging, and luminescent imaging.

One innovation described herein is the use of fluorocarbon nanoemulsion for nuclear imaging. Use of fluorocarbon as oxygen-carrying blood substitutes has gained regulatory approval in clinic practices since 1980s. Microbubbles made of fluorocarbon are also widely used for contrasted ultrasound imaging of cardiovascular diseases in clinic, with multiple products in the market. More recently, use of fluorocarbon emulsion as a tracer for fluorine-19 magnetic resonance imaging (FMRI) has gained considerable momentum, both in preclinic studies and in clinic trials. However, their implication in other imaging modalities, including nuclear imaging, has yet to be explored. In current setups, a relatively large amount of fluorocarbons, either as blood substitute or imaging agents, needs to be employed to achieve desired therapeutic or diagnostic efficiency. While fluorocarbons are inert and generally regarded safe, side effects have been observed at higher dose, including flu-like symptoms (e.g., fever, chill, and headache), skin flushing, enlargement of liver and spleen. While the newly developed FMRI has the advantage of background-free ‘hot-spot’ imaging, it requires hardware modifications of commercial MRI scanners and prefers high-field MRI, which are yet available in clinics yet due to cost and safety concerns. On the other hand, nuclear imaging like PET and SPECT has a much higher sensitivity than ultrasound and MRI, while also allowing whole-body scanning in a reasonable timeframe. The compositions and methods described herein will significantly reduce the dose of fluorocarbons administered, thus enhancing the safety profile of the agent. The disclosure also provides methods for producing a composition comprising both fluorocarbon and radioisotope that can be used for nuclear imaging (e.g., PET and SPECT) or magnetic resonance imaging (e.g., FMRI) or both.

The instant disclosure provides a method for producing fluorocarbon emulsions for diagnostic or therapeutic use. Many techniques can be implemented to produce such emulsions including, without limitation, high-pressure homogenization, extrusion, rotor-stator homogenization, ultrasonication, high-speed blending, membrane emulsification, microchannel emulsification, vortex, stirring, membrane filtration, pressure filtration and others. Probe sonication or high-pressure homogenization or both can be used to produce the emulsion. To produce emulsion with high stability and long shelf-life, for example, the at least one surfactant and additives are used. Examples of surfactants include but not limited to: egg lecithin (other names: egg yolk phospholipids, L-α-Lecithin, L-α-Phosphatidylcholine, 1,2-Diacyl-sn-glycero-3-phosphocholine, 3-sn-Phosphatidylcholine), soybean lecithin, sunflower oil, DSPE (other name: 1,2-Distearoyl-sn-glycero-3-phosphorylethanolamine), DSPE-PEG2000 {other names: DSPE-PEG2k, DSPE-mPEG2000, 18:0 PEG2000 PE, 1,2-distearoyl-sn-glycero-3-phosphoethanolamine-N-[methoxy(polyethylene glycol)-2000]}, DSPE-PEG3000, DSPE-PEG5000, DPPE, DPPE-PEG, cholesterol, Kolliphor EL (other name: Cremophor EL, Polyoxyl 35 hydrogenated castor oil), mannitol, CH₃—(CH₂)₅—(CF₂)₅—CF₃, Zonyl FS-300, Pluronic F68, Pluronic F127, Tween 20 (other name: polysorbate 20), Tween 80 (other name: polysorbate 80) and others. In some embodiments, surfactants comprising egg lecithin, DSPE-PEG2000 and cholesterol are used to make the emulsion.

The emulsions contemplated herein can be any suitable emulsions, such as oil-in-water emulsions with droplets having a diameter from about 5 nm to about 500 nm, such as form about 5 nm to about 100 nm, about 50 nm to about 150 nm, about 25 nm to about 250 nm, about 100 nm to about 350 nm, about 5 nm to about 50 nm, about 250 nm to about 450 nm, about 100 nm to about 200 nm, or about 20 nm to about 120 nm. The droplet diameter can be from about 100 nm to about 200 nm in size. Size and distribution of the emulsion can be determined by many ways, such as dynamic light scattering (DLS), laser diffraction, size exclusion chromatography, diffusion nuclear magnetic resonance, field flow fractionation, particle tracking analysis, centrifugal sedimentation, atomic force microscopy, electron microscopy and others. In some embodiments, the size and distribution of emulsion is determined by dynamic light scattering. Size of the emulsion can be tuned by many ways. For example, increasing the sonication power or processing pressure generally decrease the size of emulsion. The percentage of each component in the recipe also affects the emulsion size. For example, increasing the ratio of fluorocarbon/surfactants generally increase the emulsion size.

The choice of surfactants for emulsifying perfluorocarbon blends, including those which incorporate chelators, include lipid-based surfactants such as egg-yolk lecithin, which may be supplemented by components such as cholesterol, synthetic lipids and detergents, stabilizers such as 1-(perfluoro-n-hexyl)decane, Cremophor and others. Alternatively, surfactants may be essentially pluronic surfactants or poloxamers, which are symmetrical nonionic block co-polymers comprised of a central hydrophobic chain of polyoxypropylene flanked by two hydrophilic chains of polyoxyethylene. The relative lengths of these chains govern the properties of the surfactant, and consequently of the emulsion. The properties of the surfactant employed in the emulsion affects the general properties of the emulsion, including the size, charge, stability both in-vitro and in-vivo, interactions with blood components and in-vivo clearance rates and biodistribution. It also affects the permeability of the surfactant layer to ions, and interactions with said ions with the surfactant layer. Perfluorocarbon emulsions are thermodynamically unstable, and after formulation the size increases through a process known as Ostwald's ripening. The primary mechanism which results in breakdown of emulsions is coalescence of droplets. The properties of pluronic surfactants may be modulated by blending different surfactants with differing hydrophobic-lipophobic balance, to ensure that the hydrophobic layer is sufficient to maintain an emulsion, yet the hydrphilic PEG layer is sufficient to maintain solubility and appropriate stability in-vivo. Pluronic surfactants may be blended whilst in solution prior to formulation of the emulsion, either by dissolving in an appropriate solvent and drying, or by mixing in solution by vortexing, sonication, or other methods.

The instant disclosure also relates to a composition comprising: a nanoemulsion comprising a fluorous phase-encapsulated radioactive metal chelate. In such compositions, the at least one fluorocarbon forms a fluorous phase and the composition is a fluorous phase-encapsulated at least one fluorous hydroxamic acid chelator. Such compositions can comprise at least one radioisotope chelated by the at least one fluorous hydroxamic acid chelator.

Encapsulation of the at least one fluorous hydroxamic acid chelator can be important because the radiometal is “kept in place” in substantially a chelated form, such that the radiometal cannot diffuse from the at least one fluorous hydroxamic acid chelator. In other words, because of encapsulation, the radiometal chelated by the at least one fluorous hydroxamic acid chelator is confined within the space of a droplet.

The disclosure also relates to kits comprising (i) at least one fluorocarbon, and (ii) at least one fluorous hydroxamic acid chelator in separate containers (e.g., glass vials). The compositions comprised in a kit can comprise any suitable additional components, such as adjuvants, preservatives, and surfactants. The compositions comprised in a kit can then be mixed appropriately at the point-of-care (e.g., a hospital or clinic). Thus, in one example, a radiometal can be added at a suitable time to the at least one fluorous hydroxamic acid chelator and the resulting composition can be combined with the at least one fluorocarbon to form a fluorous phase-encapsulated radioactive metal chelate. The fluorous phase-encapsulated radioactive metal chelate can then be administered to a subject, whether the subject is a human subject or a plurality of cells (e.g., a cell culture).

As used herein, the term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

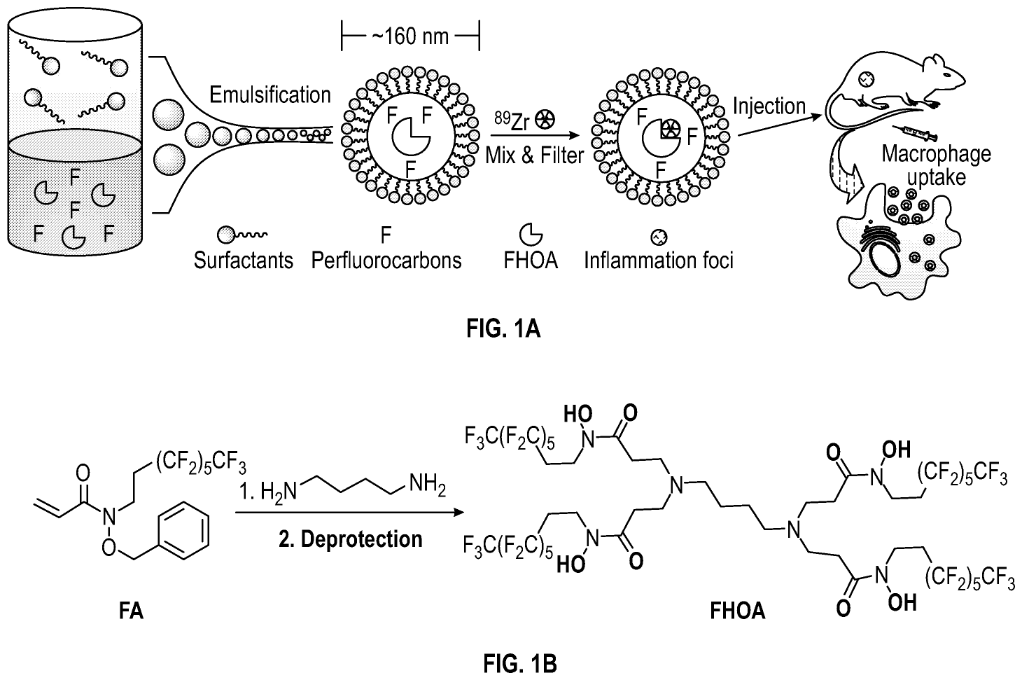

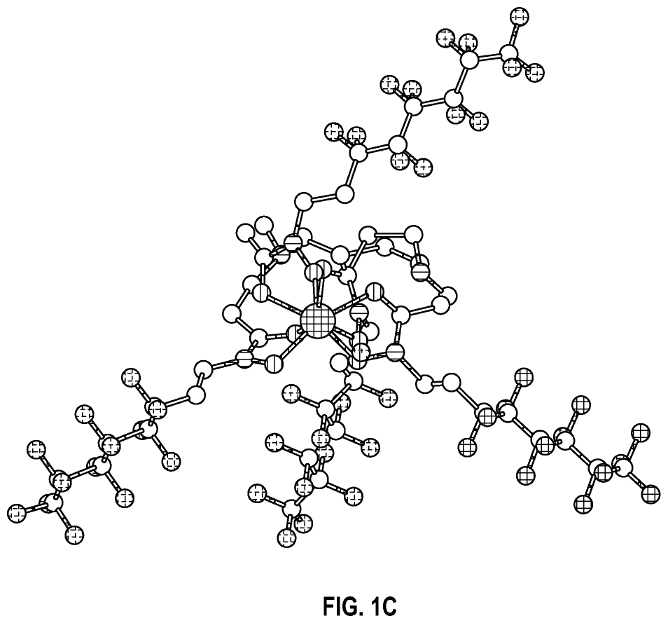

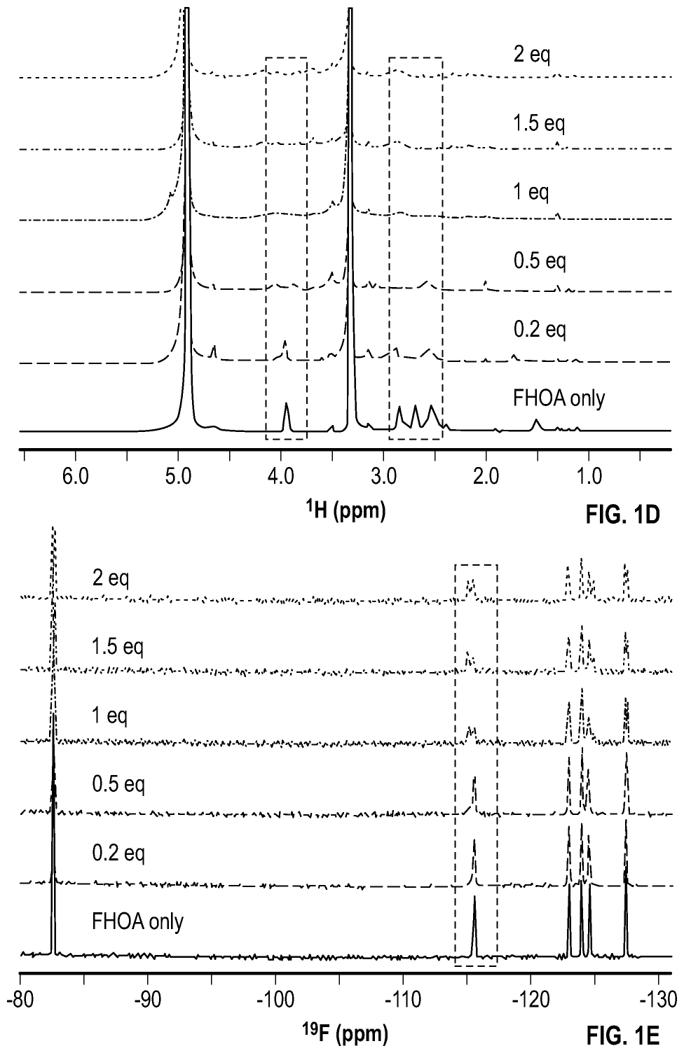

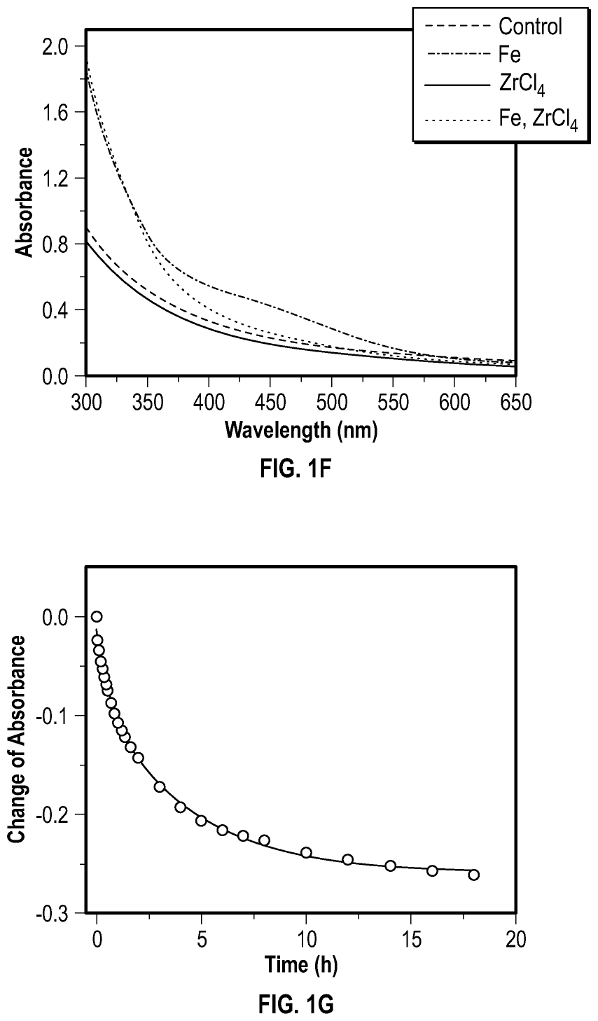

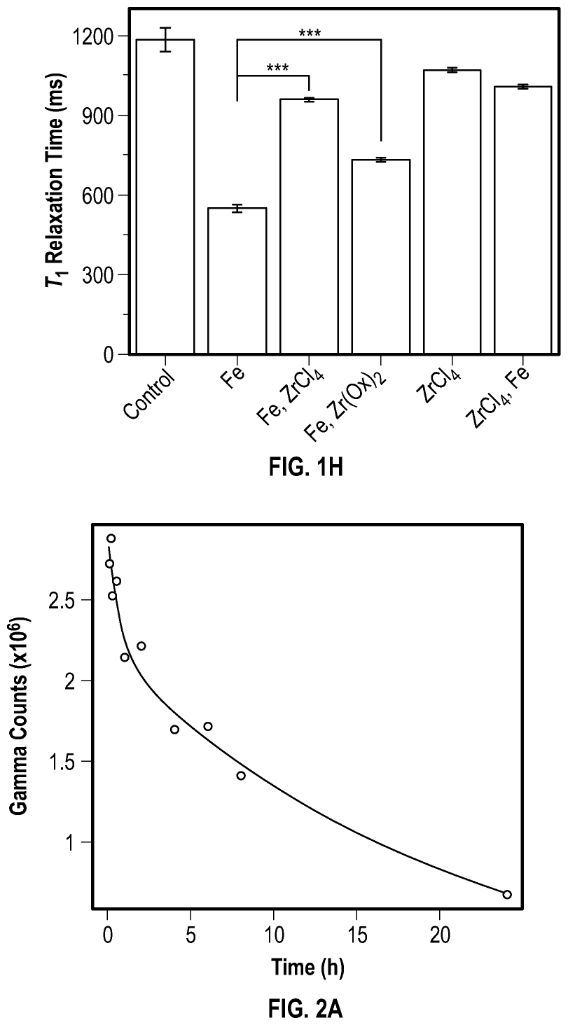

The radioactive metal used in the compositions described herein can be any suitable radioactive, including ⁸⁹Zr. Thus, for example, to enable PET macrophage detection, the radioactive isotope ⁸⁹Zr can be incorporated into a fluorocarbon composition to make a nanoemulsion. See FIG. 1A. ⁸⁹Zr has a relatively long half-life (3.3 days) matching the organ-retention time of many fluorocarbons used in biomedicine (e.g., perfluorooctylbromide, PFOB, half-life 5.1 d); this isotope is widely used in clinical trials to label monoclonal antibodies for PET. Surface attachment of ⁸⁹Zr chelate to the nanoemulsion surface can lack stability when droplets are endocytosed and trafficked to low pH lysosomal compartments resulting in metal release, cell efflux due to an osmolality gradient, and non-specific cell uptake. Thus, encapsulation of the radiometal in the nanoemulsion's fluorous phase can be a desirable via a suitable chelate. The highly hydrophobic nature of fluorocarbons helps exclude competition from water, cations, lipids and proteins that may contribute to the dissociation of ⁸⁹Zr from the carrier. Nanoemulsions formulated with chelate have a long shelf-life, and radiolabeling prior to use minimizes radiation-intensive steps and technical demands for potential clinical trial use.

By way of example, the nanoemulsions described herein can be prepared using any suitable chelator, such as the octadentate chelator, fluorous hydroxamic acid (FHOA), which can be synthesized or obtained from any suitable source. The FHOA binds Zr⁴⁺, which is a hard Lewis acid with high oxophilicity. FHOA was prepared at gram scale using an Aza-Michael reaction between a fluorous acrylamide (FA) and primary diamine (FIG. 1b). FHOA shares the hydroxamic acid units of the hexadentate desferroxamine (DFO), a well-known iron chelator that also strongly binds Zr⁴⁺ and is used in immuno-PET. The FHOA chelate provides eight oxygen coordination sites to saturate the Zr⁴⁺ sphere to avoid labile binding with H₂O and biomolecules, which are speculated to be a source of Zr-DFO instability. Force field simulation (Molecular Mechanics MM2) shows the formation of a distorted square antiprismatic complex (FIG. 1C), with an averaged Zr—O length of 2.1 Å, close to the 2.2 Å calculated from the X-ray structure of Zr-tetrahydroxamate. FHOA has a fluorine content of 54.26% and is soluble in PFOB up to 15 mM (at room temperature) and perfluoro-15-crown-5-ether (PFCE) up to 2 mM with mild heating; both of these fluorocarbons have been widely used for FMRI. Titration of ZrCl₄ (non-radioactive) into FHOA in solution causes attenuation and shifting of FHOA peaks in ¹H (FIG. 1d) and ¹⁹F nuclear magnetic resonance (NMR) (FIG. 1E); no peak change is observed beyond 1 eq, suggesting a 1:1 ratio for Zr-FHOA binding (FIG. 1E). Binding of FHOA to ZrCl₄ (1 eq) in solution is rapid, with completion in <20 min, as seen via the change in ¹⁹F NMR spectra over time (data not shown).

When the chelator used to prepare the compositions described herein, the FHOA can be dissolved in the fluorocarbons before formulating into aqueous FERM nanoemulsions using suitable lipid surfactants described herein and microfluidization as described in the Examples herein. Prepared lipid PFOB and PFCE nanoemulsions can have similar diameters ˜160 nm with polydispersity <0.1, as measured by dynamic light scattering (DLS). Inclusion of FHOA (1 or 10 mM) has no statistically significant impact (p>0.05) on the nanoemulsion size (Table S1) or stability over at least two months.

Among various biologically relevant metals, Fe³⁺ is the strongest competitor for Zr⁴⁺ chelate translocation in FERM nanoemulsion, as is the case for widely used DFO and hydroxypyridinonate. Similar to DFO, FHOA binds Fe³⁺, but with a mixed stoichiometry of 1:1 and 1:2 Fe:FHOA, as revealed by liquid chromatography mass spectrometry (LC-MS). Addition of Fe³⁺ to FHOA in PFOB (10 mM in oil) nanoemulsion causes an increase in UV-Vis absorption at ˜450 nm, which is then reduced by addition of Zr⁴⁺, suggesting Fe³⁺ displacement by Zr⁴⁺ (FIG. 1F). Extinction of absorption at 450 nm was monitored over a 18 h period and fitted, yielding bi-exponential decay half-lives of 0.32±0.07 h and 2.91±0.26 h (FIG. 1E). To further assess Fe³⁺ binding competition, we measured the change in the ¹⁹F spin-lattice relaxation time (T₁) in the nanoemulsion upon metal binding. FeCl₃ (2 eq) added to FHOA in PFOB nanoemulsion caused a 53% drop in ¹⁹F T₁ (from 1.2 s to 0.6 s) due to the paramagnetic relaxation enhancement mechanism. Subsequent addition of ZrCl₄ (2 eq) recovers T₁ to 0.96 s via the displacement of strongly paramagnetic Fe³⁺ with Zr⁴⁺ (FIG. 1H). Notably, use of an aqueous suspension of Zr oxalate (2 eq, non-radioactive), a widely used but less reactive form of ⁸⁹Zr, also displaces Fe³⁺ and recovers T₁ to 0.73 s (FIG. 1H). Conversely, addition of excess (>2 eq) Fe³⁺ to Zr-saturated nanoemulsion only decreases T₁ by 5.7% (FIG. 1h), as Fe³⁺ is unable to displace all Zr⁴⁺ from FHOA. These data indicate that Zr⁴⁺ forms a more stable complex with FHOA than Fe³⁺.

The compositions described herein can be preformed prior to use or formed at the point of care (e.g., at an imaging clinic). For example, preformed PFOB or PFCE nanoemulsions (1 mL) containing chelate, and formulated with lipid-based surfactants, can be radiolabeled by mixing with ⁸⁹ZrCl₄ (in 1 M HCl) at room temperature for 3 hours as described herein. Unbound ⁸⁹Zr was removed by a single gel filtration step, resulting in a radiochemical yield of 63.2±6.5% in 0.8 mL elution. A simple mixing is adequate without the need for heating or pH adjustment. We used pre-packed Sephadex-25 gravity columns (GE healthcare, Pittsburgh, PA) for purification due to their commercial availability and well-established protocols enabling rapid purification within 5 min, although other self-packed resins (e.g., Sephadex-100) may net higher yield. We also tested nanoemulsion radiolabeling using ⁸⁹Zr-oxalate (in 1 M oxalic acid), where unlike ⁸⁹ZrCl₄, neutralization was necessary to achieve efficient labeling. Radiolabeling yield of FERM is 50.9±4.7% (pH=6.5) with ⁸⁹Zr-oxalate 3 h after labeling. As a substitute for lipid surfactant, efficient radiolabeling of nanoemulsion was also achieved with a triblock copolymer surfactant, Pluronic F68, with ⁸⁹ZrCl₄ yields of 58±7.1% for FERM. Overall, FERM nanoemulsion radiolabeling is flexible with respect to surfactant type, as it relies on an encapsulated chelate in the inert fluorocarbon core.

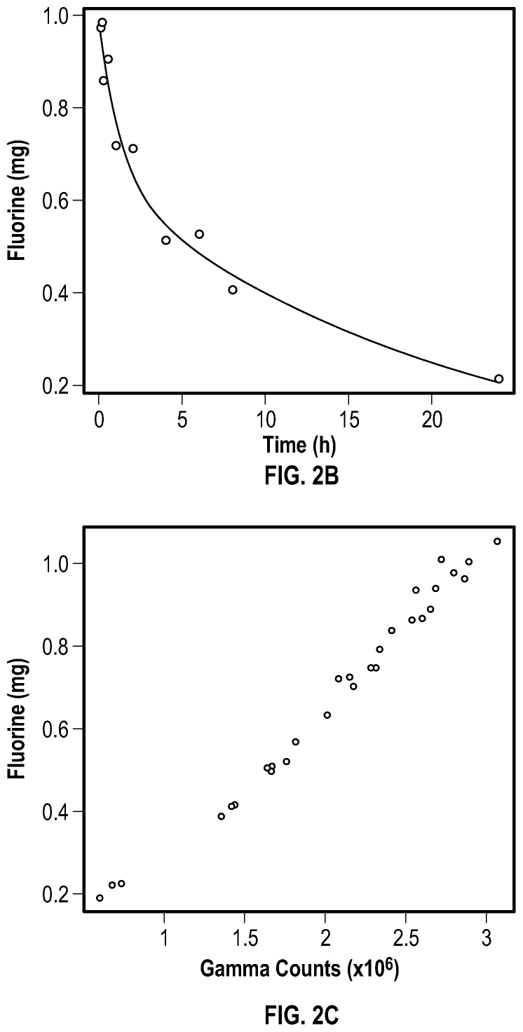

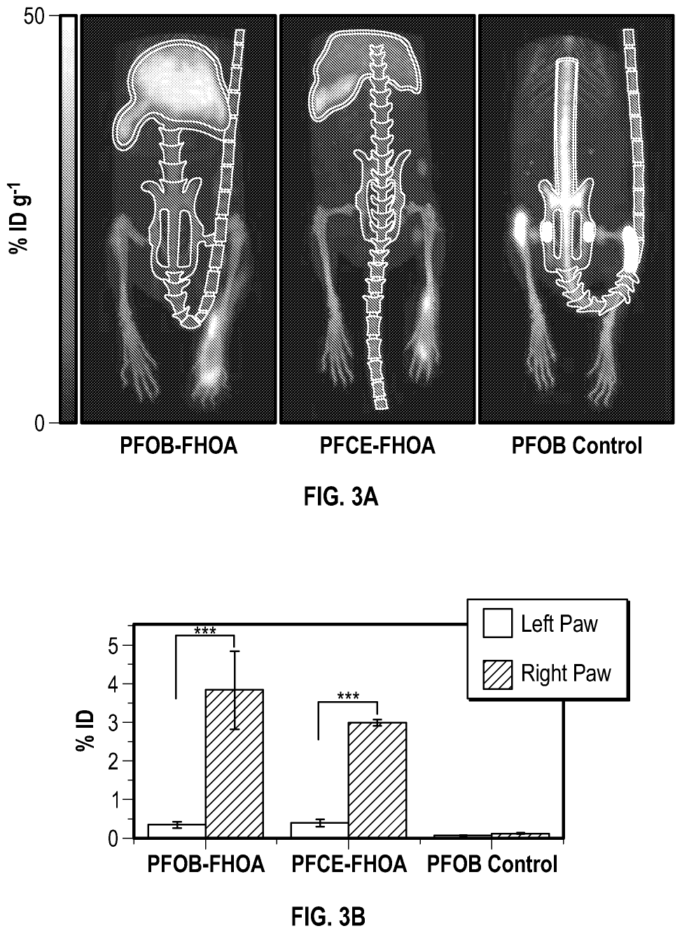

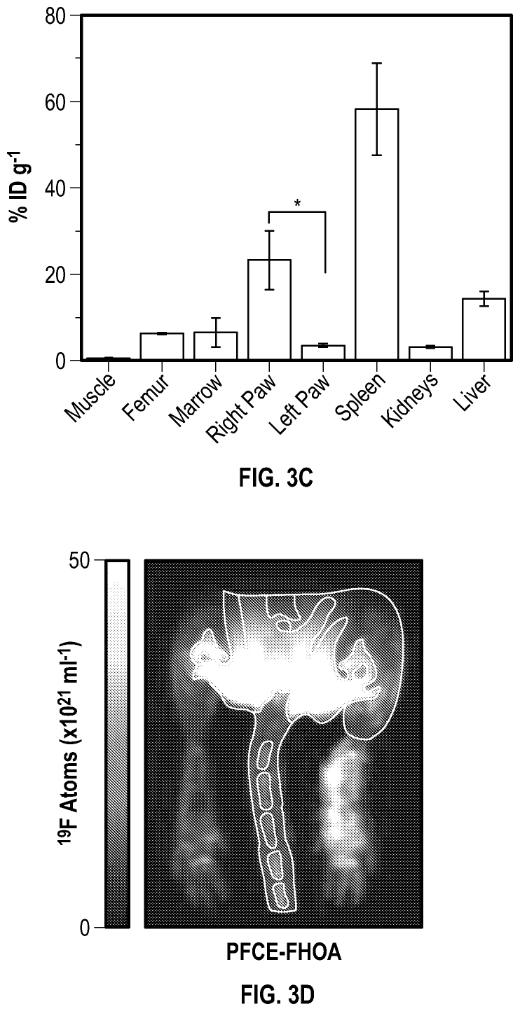

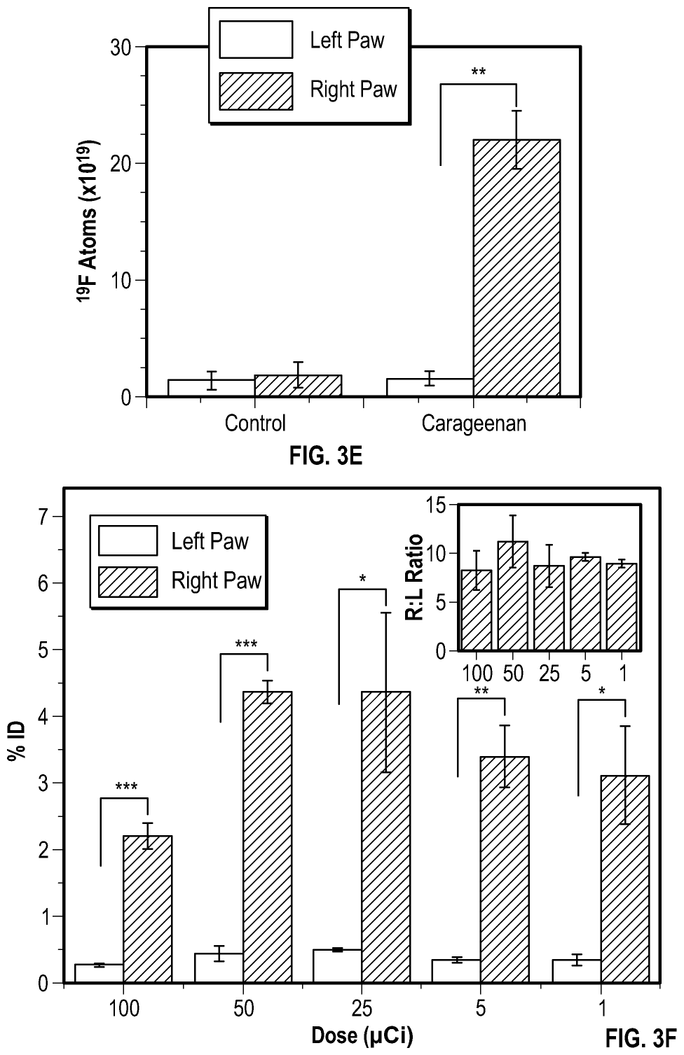

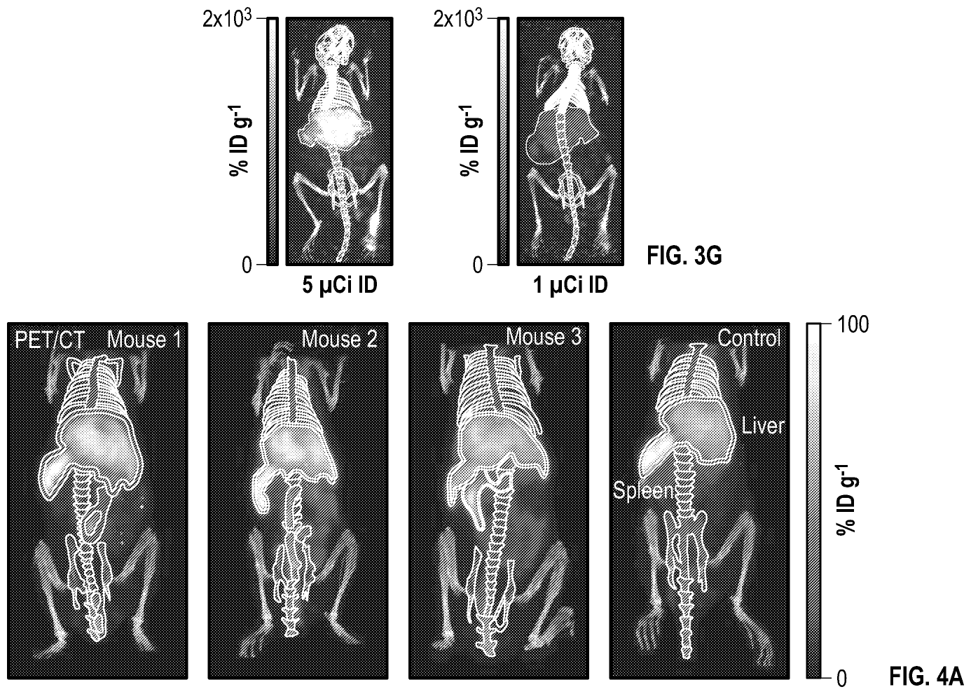

FERM nanoemulsions are versatile preclinical inflammation probes for a wide range of diseases. The feasibility of using PET-FMRI inflammation detection in an acute inflammation rodent model is an example of an application of the compositions described herein. The model uses an injection of A-carrageenan plant mucopolysaccharide (50 μL, 2% in saline) into the footpad of mice, which results in visible swelling at the injection site and is commonly used to test anti-inflammatory drugs and immune response. Radiolabeled ⁸⁹Zr FERM nanoemulsion was injected (n=3, 0.2 mL, 100 μCi, ˜6×10²⁰ F atoms) by tail vein 1 h after carrageenan treatment. PET/CT images were acquired 24 h thereafter to permit FERM uptake by the RES, including macrophages, based on the empirical results of the slow phase blood clearance half-life (14.5 h, FIG. 2). In PET, mice receiving FERM, formulated with either PFOB or PFCE, display hotspots in the inflamed right paw, with little observable signal on the contralateral side (FIG. 3A). Immunohistochemical results in the hind paws show macrophage fluorocarbon uptake in situ following intravenous infusion, consistent with prior studies in various inflammation models. Prominent signals are also observed in spleen and liver (FIG. 3A), consistent with RES clearance. Control animals received tail vein injections of either free ⁸⁹ZrCl₄ or ⁸⁹Zr treated nanoemulsion without chelate, and both display similar trafficking patterns (FIG. 3A, right); free ⁸⁹Zr is taken up by bone, especially the vertebral column and knees.

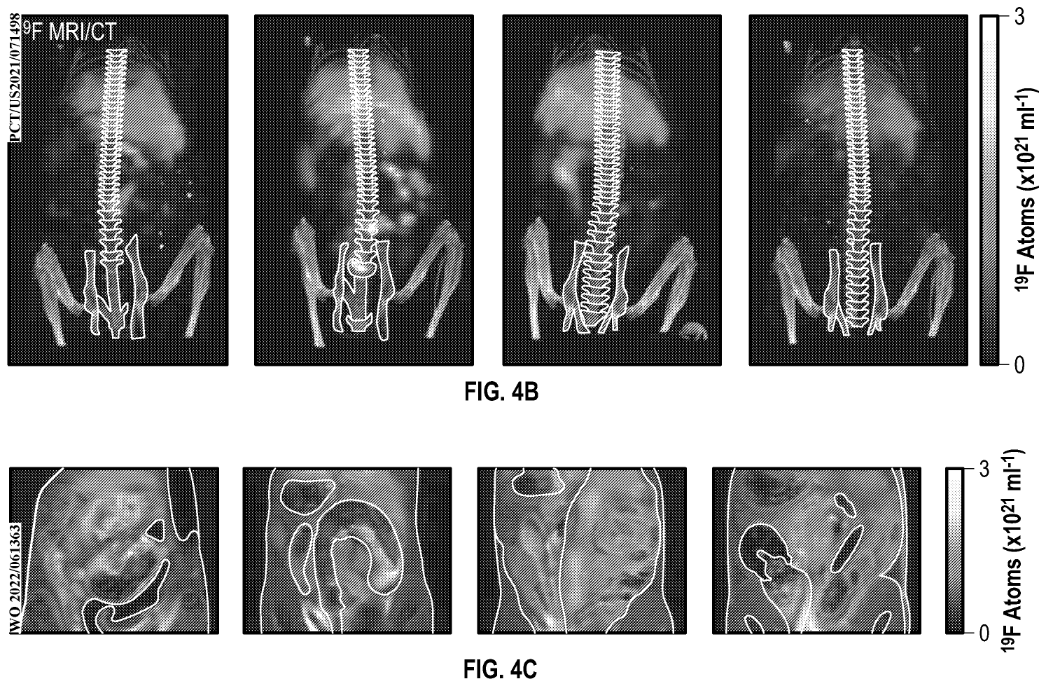

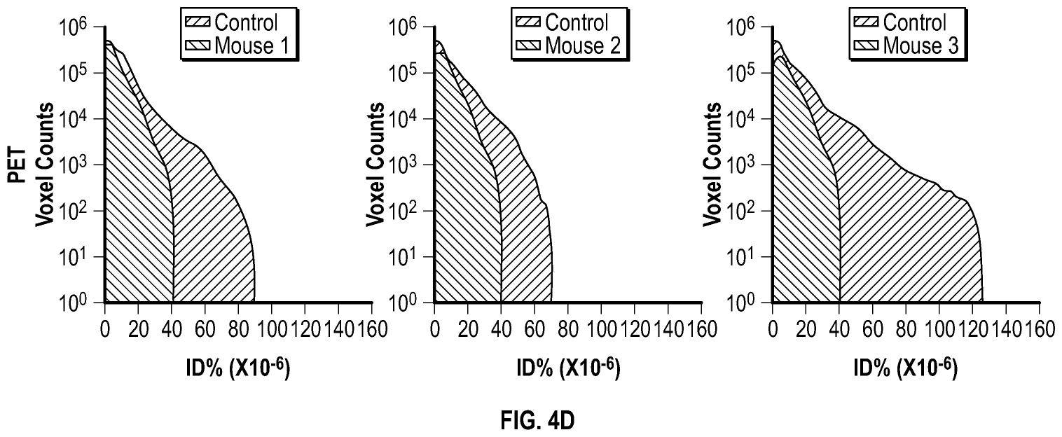

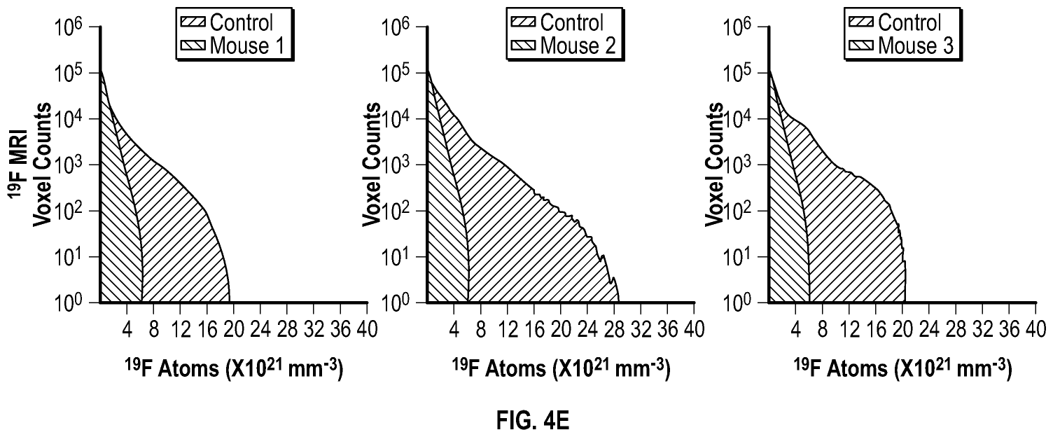

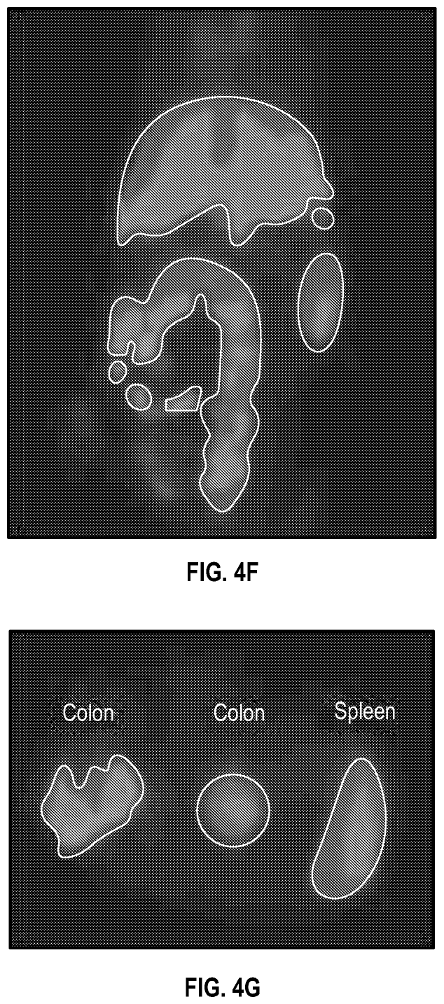

The compositions described herein can also be used to image, for example, IBD. FIG. 4 shows PET-FMRI suing the compositions described herein in a mouse model using (FIG. 4). The IBD model was induced by adding dextran sulfate sodium (DSS) to drinking water for C57BL/6 mice, resulting in ulcerative colitis-like inflammation, with prominent inflammatory infiltrates, including macrophages in the gastrointestinal tract. IBD clinical symptoms peak at approximately eight days after the start of DSS treatment. At peak, a single intravenous injection of ⁸⁹Zr FERM nanoemulsion (n=5, 0.2 mL, 100 μCi, ˜6×10²⁰ F atoms) was administered, and 24 h thereafter ⁸⁹Zr PET/CT and ¹⁹F/¹H MRI data were acquired. Representative images are shown FIGS. 4A and 4B, where both the PET and FMRI data are co-registered to CT. Major hotspots are observed in the colon in ⁸⁹Zr PET images for all IBD mice. IBD lesions are patchy and heterogenous among subjects and distributed in ascending and descending colon. Control mice, without DSS induction, display prominent uptake in liver and spleen with minimal colon signal, as expected for RES clearance of FERM nanoemulsion. FMRI in the same animals also display inflammatory hotspots in colon (FIG. 4B). Generally, FMRI lesion signals are more punctate compared to the relatively diffuse PET signals. Overlays of ¹⁹F and high-resolution ¹H anatomical images show ¹⁹F signal localization in the anatomical context of the colon wall (FIG. 4C). To quantitate inflammation in bowel, ROIs were placed around peritonea, and the resulting signal histograms for PET and FMRI are displayed in FIGS. 4D and 4E. Both methods clearly show a much larger proportion of high-signal voxels in the IBD mice in comparison to controls. Notably, the anatomical signal patterns for PET and FMRI are largely overlapping (FIGS. 4F and 4G).

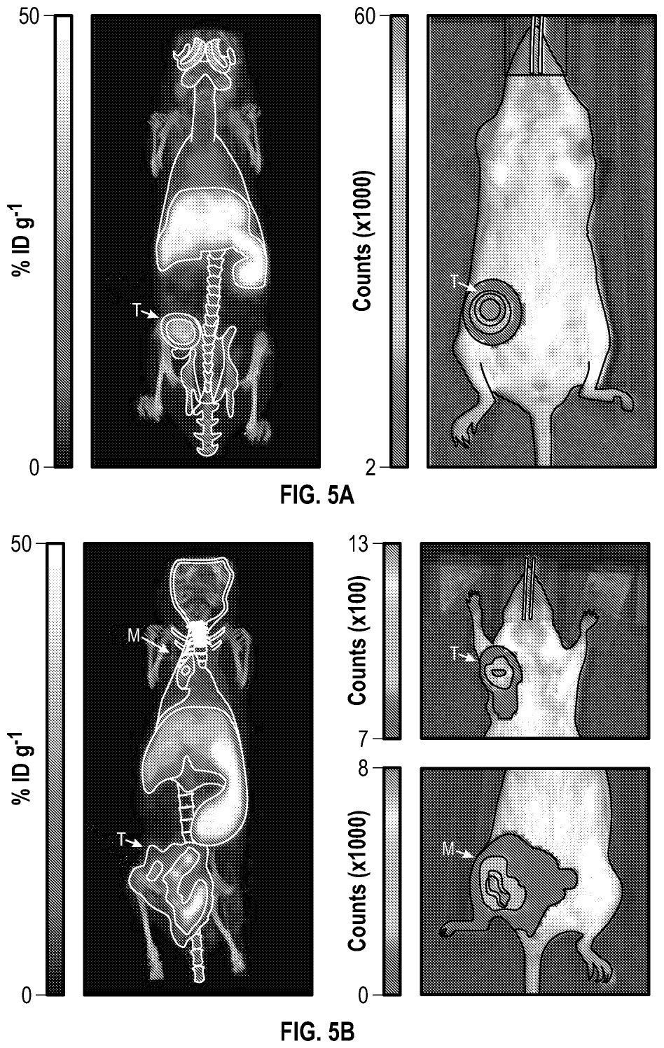

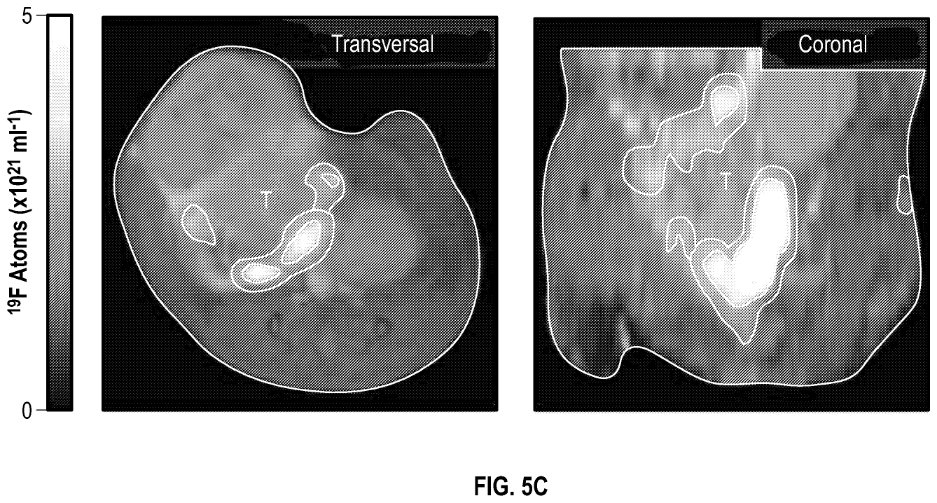

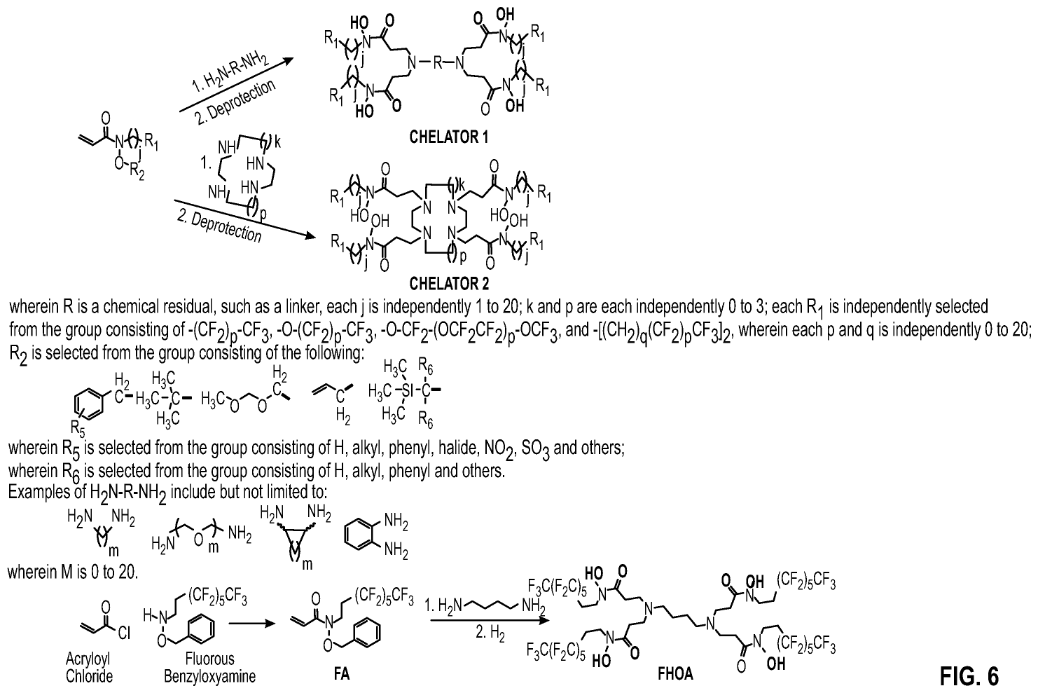

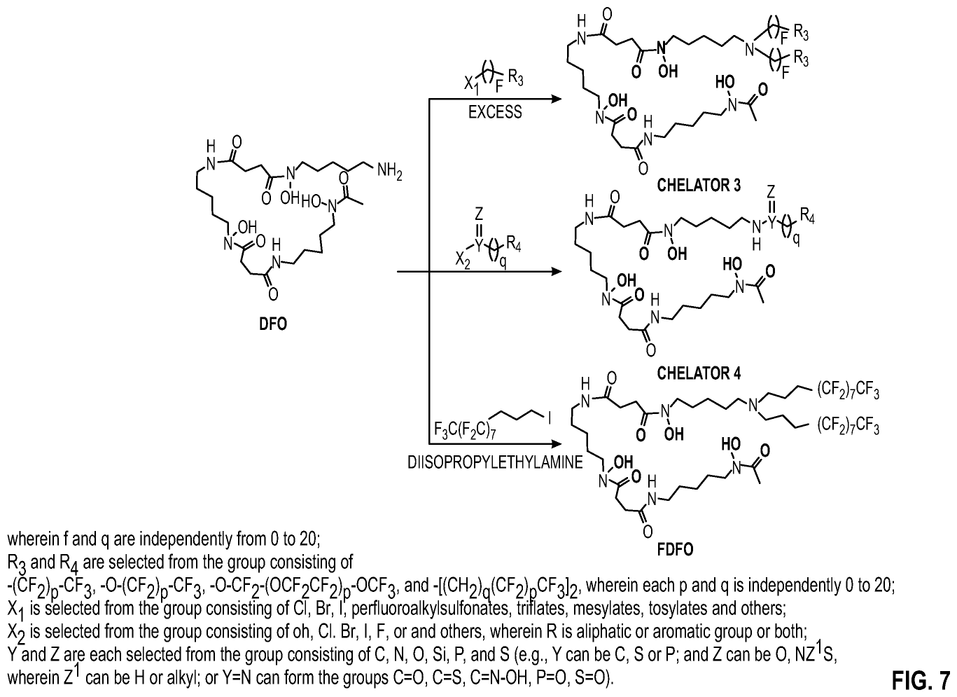

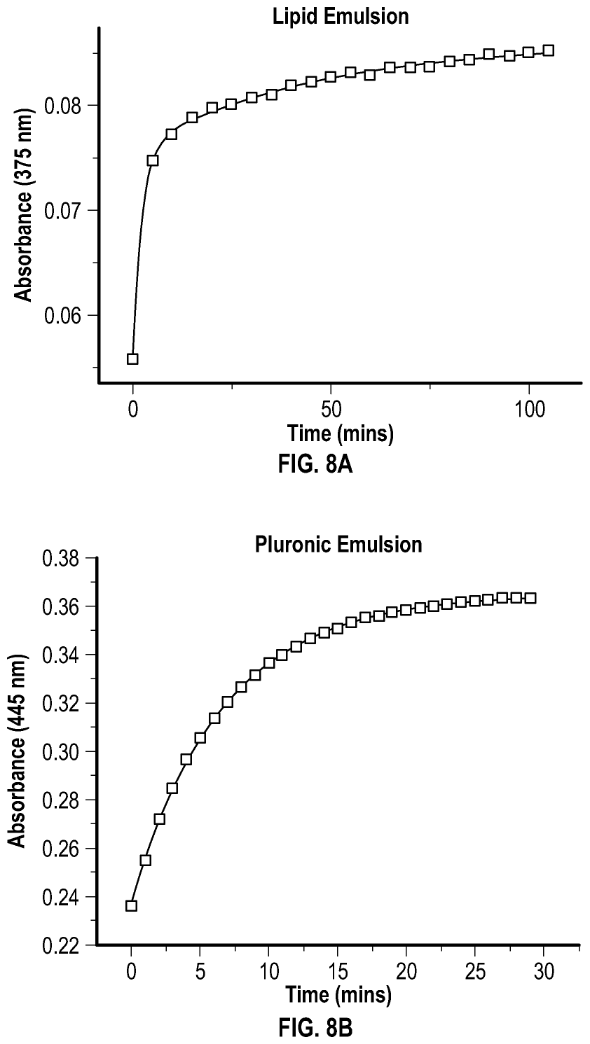

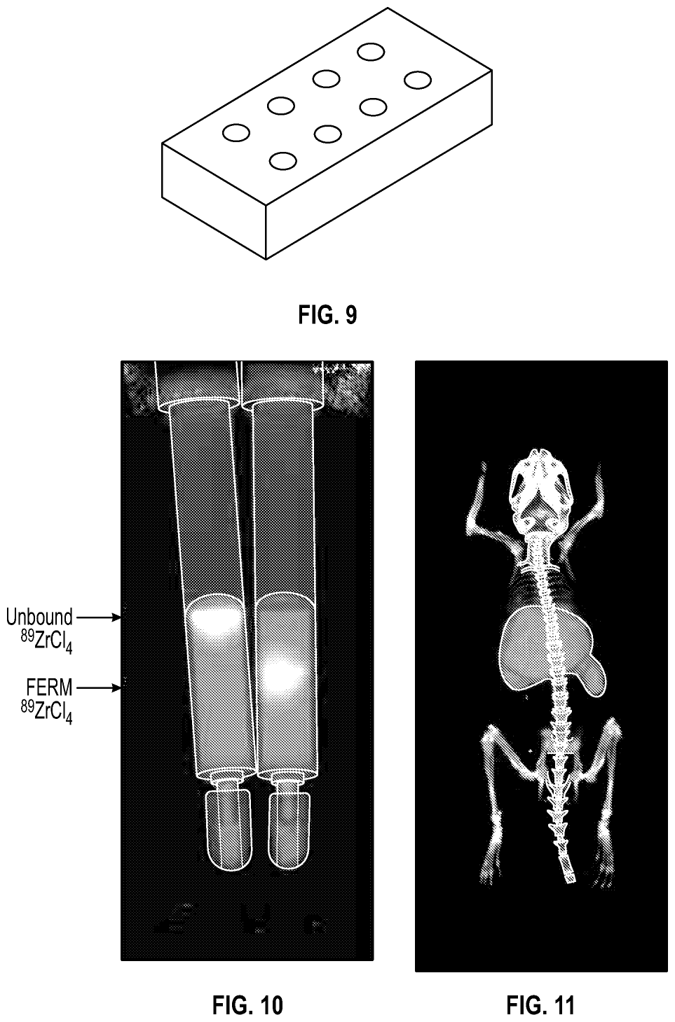

The compositions described herein can also be used to tumor associated macrophages (TAMs), which play a central role in the initiation, progression, and metastasis of tumors, and their density in the tumor microenvironment is often associated with tumor aggressiveness and patient survival rate. Imaging TAMs and metastasis-associated macrophages may enable early malignancy detection, as well as response assessment of immunotherapies. Thus, we investigated the use of FERM nanoemulsion for PET-FMRI macrophage imaging in a breast cancer mouse model (FIG. 5). Tumor cells (4T1) expressing luciferase were implanted in mammary fat pad, and bioluminescence imaging (BLI) confirmed primary tumor growth in flank (FIG. 5A). FERM nanoemulsion PET-FMRI scans were conducted at 2 weeks (“early”, n=5) or 5 weeks (“late”, n=5) post-implantation in separate cohorts. At 24 h prior to imaging, animals received intravenous ⁸⁹Zr FERM nanoemulsion (0.2 mL, 100 μCi, ˜6×10²⁰ F atoms). In the early cohort, PET images (FIG. 5A) display major hotspots in the whole tumor area, as well as in the liver and spleen. In the late group, FERM has significant presence in the flank tumor periphery for both PET (FIG. 5B) and FMRI (FIGS. 5C and 5D), with minimal signal in the tumor center, consistent with peripheral macrophage infiltration and tumor core necrosis. Gamma counting of the excised tumors confirm high tumor uptake of the agent, with 5.3±1.8% ID in early tumors and 10.7±3.6% ID in late tumors. Notably, putative metastases are observed in the axillary lymph node region, visible with both PET (FIG. 5B) and BLI (FIG. 5E), in 2 out of 5 animals in the late cohort. In the late cohort lungs, we observe slightly elevated, diffuse PET signal (n=5), which was not apparent in FMRI or BLI.

The compositions described herein can be administered in a single dose or in multiple doses, as necessary. For example, a single dose of FERM nanoemulsion enables multimodal FMRI and PET/CT detection in the same subject. For dual-mode imaging, PFCE, with 20 equivalent fluorine atoms, can be used to prepare the compositions described herein to, among other things, simplify FMRI acquisition methods. In the acute inflammation model, in vivo spin density-weighted ¹⁹F and T₂-weighted ¹H multi-slice images were acquired, followed by PET/CT scans in the same mice, where a stereotaxic mouse holder was used to maintain animal position across the imaging platforms. Both FMRI and ⁸⁹Zr PET scans display hotspots in the inflamed right paw (FIG. 3D), with colocalization of ¹⁹F and ⁸⁹Zr signals, and minimal signals in the contralateral paw. Quantification of both ¹⁹F FMRI and ⁸⁹Zr PET hotspots in right paw display >10-fold higher signal compared to contralateral paw (FIG. 3E).

The compositions described herein (e.g., ⁸⁹Zr FERM nanoemulsion) can be used in various methods, including methods for imaging inflammatory disease. The compositions described herein can be used for imaging with high specificity, sensitivity, and versatility, using both PET and FMRI. For example, the fluorous ⁸⁹Zr chelator FHOA effectively encapsulates the radioisotope into the nanoemulsion core. This strategy minimizes radioisotope leakage and non-specific cell labeling and allows for flexibility in surfactant design. FHOA was prepared and purified in gram scale in a single run. FERM can be formulated as a cold nanoemulsion (e.g., without a radioactive nuclide), preloaded with chelate, and can display long term stability (≥2 months). Before intravenous delivery, FERM can be radiolabeled with, e.g., ⁸⁹Zr via simple premix and filtration steps. The use of ⁸⁹Zr with its relatively long half-life (3.3 d) allows for RES cell (macrophage) uptake of probe, as well as longitudinal studies over several days with a single administration.

In vivo stability of FERM nanoemulsions evaluated by blood pharmacokinetic analysis has shown that such compositions exhibit minimal dissociation in vivo. Both dosimetry and ¹⁹F NMR intensity analyses of serial blood samples display bi-exponential decreases over time; the slow time constant (˜15 h), presumed to be RES uptake, shows good agreement between the two measurement methods (>0.99 correlation).

Thus, for example, ⁸⁹Zr and ¹⁹F signal generators stay co-complexed during the RES uptake period. The fast-phase time constant, representing the initial blood perfusion of agent, varies when measured by dosimetry (t_{1/2}=36 min) and ¹⁹F (t_{1/2}=54 min, p<0.05). Variations in blood sample timing between the two methods and γ-counting times (˜1-4 min) of each sample could possibly explain the discrepancy. Moreover, gel-filtration purification of the compositions described herein can be imperfect, with ˜1% of free ⁸⁹Zr eluted with FERM (0.8 mL), as tested by loading ⁸⁹ZrCl₄ (0.5 mCi in 1 mL H₂O, n=2) directly into the column. The free ⁸⁹Zr has a short circulation time and quickly deposits into the skeleton, resulting in a higher blood distribution rate in dosimetry. Residual ⁸⁹Zr after filtration could be further minimized by improved resins for purification or by high-performance size-exclusion chromatography. Although FHOA is a potent chelator, there may also be some degree of ⁸⁹Zr bound to lipid headgroups of the surfactant used in the in vivo studies. However, there are alternative biocompatible surfactants that can minimize potential residual surface adherence, such as Pluronic F68, as described herein.

Prior preclinical studies have used FMRI with fluorocarbons for macrophage labeling in situ, for example in myocarditis, neuroinflammation, solid organ transplant rejection, IBD, as well as various cancer models. Importantly, these same studies firmly establish intracellular tissue macrophage uptake of fluorocarbon agent as the dominate image signal observed in vivo. For example, IBD studies have shown exclusive colocalization of fluorescently-conjugated fluorocarbon nanoemulsion and F4/80+ macrophage in immunohistochemistry micrographs, as well as correlation between histopathology quantification of lesion burden in colon wall to total ¹⁹F signal in the same specimens. Additionally, quantitative PCR analysis correlating macrophage burden via CD68 RNA levels with ¹⁹F signal in colon samples show a linear relationship. Moreover, clodronate liposome treatment ablates the ¹⁹F signals in the colon of IBD mice. In solid tumor models, immunohistochemistry shows specific uptake of fluorescent fluorocarbon nanoemulsion in macrophages both at primary tumor periphery at late stages and at metastasis sites. While fluorocarbon nanoemulsions have been widely studied for FMRI, its description for use as a PET tracer has been limited.

The FERM nanoemulsions described herein show promise for precise detection of a broad range of inflammatory lesions with high macrophage specificity. In the case of IBD, resident macrophages in the colon play a key role in the homeostasis of the bowel, and macrophages derived from blood monocytes are important mediators of chronic inflammation in IBD along with Th1 and Th2 type T cells. The ‘gold-standard’ IBD test is colonoscopic biopsy, an invasive procedure requiring multiple tissue bites for diagnosis, which may result in sampling errors and cause patient discomfort, thus driving the need for more precise diagnostics for staging and monitoring treatment course. Physiologic bowel uptake of ¹⁸F-FDG is highly variable in the colon and can be quite intense, especially in patients on Metformin, thus limiting FDG's usefulness. Oncology also presents another major area of use for FERM for precision macrophage imaging due to the diagnostic potential, as well as the increasing focus on macrophages as therapeutic targets.

In both PET and FMRI (FIGS. 3-5), lesion foci are the only major hotspots other than the liver and spleen. Hotspots display anatomical similarities across PET and FMRI modalities, indicative of FERM stability in vivo. The MRI-apparent lesions appear as more punctate compared to the more diffuse PET detected lesions. There are fundamental differences between the two imaging techniques with regards to intrinsic sensitivity and resolution, image reconstruction methods, point-spread functions, and partial-volume effects that impact quantification and small lesion appearance. Generally, FMRI is prone to false negative signals due to sensitivity limitations, whereas high-sensitivity PET imaging is prone to false positives, thus a bimodal readout provides a complementary representation of the ground-truth lesion macrophage distribution using FERM nanoemulsion. There may be effective ways to computationally amalgamate the two data sets to obtain a closer ground-truth representation of the lesion distribution, and this is the subject of future investigations. As a practical matter, the ¹⁹F is advantageous as a stable tag to assay probe biodistribution via ¹⁹F NMR of tissue samples, as well as the fate of the ⁸⁹Zr+fluorocarbon complex when combined with γ-counting measurements (e.g., FIG. 2).

While PET and FMRI using separate instruments, future advancements in imaging hardware may be feasible to enable simultaneous acquisition of PET-FMRI data. Recently, dual-mode PET and ¹H-only MRI scanners have been in clinical service, for example, in cardiology, oncology and neurology. These advanced scanners are advantageous for accelerated data acquisition, improved image registration, and motion correction of voluntary and involuntary movements like respiration and bowel peristalsis; in the future, these beneficial features could be utilized by a composite PET, ¹⁹F/¹H MRI scanner. Whole-body clinical PET to identify putative lesions, followed by inflammation hotspot ¹⁹F/¹H MRI in a smaller field of view with high soft-tissue resolution, may yield a rich dataset for treatment planning and response monitoring.

The compositions described herein, can further comprise one or more pharmaceutically acceptable carriers, diluents, excipients or combinations thereof. The compositions described herein can be formulated for administration one or more of a number of routes, including but not limited to buccal, cutaneous, epicutaneous, epidural, infusion, inhalation, intraarterial, intracardial, intracerebroventricular, intradermal, intramuscular, intranasal, intraocular, intraperitoneal, intraspinal, intrathecal, intravenous, and the like.

A “pharmaceutical excipient” or a “pharmaceutically acceptable excipient” comprises a carrier, sometimes a liquid, in which an active therapeutic agent is formulated. The excipient generally does not provide any pharmacological activity to the formulation, though it may provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “pharmaceutically acceptable carrier” or “excipient” includes any and all solvents, dispersion media, coatings, antibacterial and antifungal agents, isotonic and absorption delaying agents that are physiologically compatible. In one example, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Pharmaceutical compositions may be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

The instant disclosure also provides compositions (e.g., compositions comprising (i) at least one fluorocarbon, (ii) at least one fluorous hydroxamic acid chelator, and (iii) at least one radioisotope) and methods for non-invasive imaging of inflammation. Inflammation is a common symptom that results from defensive innate immune response toward invasive stimuli and features upregulation of immune cells. Though it promotes pathogen clearance and tissue recovery, uncontrolled inflammatory response plays a major role in many diseases, including atherosclerosis, arthritis, asthma, allergy, coeliac disease, glomerulonephritis, sarcoidosis, inflammatory bowel disease (IBD), and cancers.

While conventional MR and CT imaging of inflammation detects anatomical damages in late stage, nuclear medicine such as PET or SPECT imaging targeting inflammation markers provides opportunities in early diagnosis and therapeutic intervention. The use of ¹⁸F-FDG (t_{1/2}=108 min) in PET imaging of many inflammatory diseases were well documented in clinical and preclinical settings. However, FDG targeting glucose metabolism has no specificity for inflammation, with strong background signals observed in brain, heart, kidney and other active muscles. For patients with diabetes, tumor or in surgical recovery, its usefulness is further limited. For example, patients on Metformin, a drug use to treat diabetes, show significantly higher FDG uptake in the bowel, which may confuse PET assessment of bowel disease, such as IBD and colonic neoplasm (Gontier, E. et al. Eur J Nucl Med Mol Imaging 2008, 35:95-99). Fluorine-18 labelled mannose, a revised version of FDG, was made with increased macrophage uptake for PET imaging of atherosclerotic plaque inflammation. Other small molecule radiotracers targeting inflammatory markers, like cytokines, translocator proteins, enzymes, and integrin receptors, have been designed with better specificity and various degrees of success.

Given the high endocytosis activity of monocytes and macrophages involved in inflammation, nanoparticles mimicking as ‘pathogens’ are natural candidates for the diagnosis of inflammatory diseases. Nanoparticle tracers administered to a subject are internalized by circulating monocytes, which then migrate to inflammation foci, allowing its visualization by imaging techniques. Currently, only a few radiolabeled nanoparticles targeting macrophages were studied for PET or SPECT imaging of inflammatory events. In one example, polyglucose nanoparticles labeled with ¹⁸F through chemical reaction on the particle surface were used for PET/CT imaging of inflammatory atherosclerosis in animal models (Keliher, E. J. et al. Nat Commun 2017, 8:14064-14076). In one example, a dextran-coated iron oxide nanoparticle labelled with ⁶⁴Cu on the particle surface was also used for PET/CT imaging of inflammatory atherosclerosis (Nahrendorf, M. et al, Circulation 2008, 117:379-387). In another example, a magnetic nanoparticle coated with NaYF₄ or Al(OH)₃ can be labelled with ¹⁸F on the surface for PET or SPECT or MR imaging of diseases, including inflammatory diseases (Cui, X. et al, Published U.S. Appl. No. 2015/0064107). Unlike the widely use surface-labelling method of nanoparticle, the present invention provides a method for labelling the core of fluorocarbon nanoparticles, by use of fluorous chelator, to achieve higher stability.

The disclosure, therefore, provides a method for imaging of inflammatory diseases comprising administering a composition comprising (i) at least one fluorocarbon, (ii) at least one fluorous hydroxamic acid chelator, and (iii) at least one radioisotope to a subject; and imaging the subject. Imaging techniques are selected from the group consisting of PET, SPECT, CT, FMRI and MRI. More than one imaging method can be used for imaging of inflammatory disease. In addition, any acquired images can be quantified to evaluate the severity or stage of inflammatory disease. Examples of said inflammatory diseases include inflammatory bowel disease (IBD), Crohn's disease, ulcerative colitis, atherosclerosis, arthritis, coeliac disease, glomerulonephritis, sarcoidosis, inflammatory allograft rejection, autoimmune diseases and others.

Imaging of inflammatory diseases comprises:
(1) preparing or providing a composition comprising (i) at least one fluorocarbon, (ii) at least one fluorous hydroxamic acid chelator, and (iii) at least one radioisotope;
(2) optionally purifying, diluting, pH adjusting or other treatments of the composition in (1);
(3) administering the composition from (1) or (2) to a subject, wherein administration method can be oral administration, intravenous injection, intramuscular injection, intraarterial injection, subcutaneous injection or others (e.g., one or more administration methods);
(4) imaging of the subject in (3) after administration of the compositions from (1) or (2) to obtain acquired images using imaging methods are selected from the group consisting of PET, SPECT, CT, FMRI and MRI (e.g., images acquired at one or more times after the administering of compositions of (1) or (2); and
(5) optionally analyzing of acquired images from (4), e.g., with the aid of a computer and software.

The acquired images can be used for many purposes. For example, the acquired images can be used to identify the location of inflammation in the subject. In addition, or alternatively, the acquired images can be used to evaluate the severity or stages of inflammation. In addition, or alternatively, the acquired images can be used to assist treatment decisions. In addition, or alternatively, the acquired images can be used to evaluate treatment efficiency. In addition, or alternatively, the acquired images can be used for drug screening (e.g., drug screening in the context of determining what drugs from a panel are more effective than others in treating inflammation in a subject).

The instant disclosure also provides compositions (e.g., compositions comprising (i) at least one fluorocarbon, (ii) at least one fluorous hydroxamic acid chelator, and (iii) at least one radioisotope) and methods for imaging cancer and cancer metastasis. The imaging techniques can be selected from the group consisting of PET, SPECT, CT, FMRI, and MRI.

Numerous agents, including ¹⁸F-FDG have been developed for the diagnosis of cancers with nuclear imaging method, such as PET and SPECT. In fact, around 97% of clinical PET/CT scans were performed in oncology to improve tumor management, such as diagnosis, staging, monitoring, and radiotherapy planning. While many drugs are designed to target tumor cells directly, some others are designed to target tumor-associated macrophages (TAMs) that reside in the tumor microenvironment. TAMs play a central role in the initiation, progression and metastasis of tumors, and their density in the tumor microenvironment is often associated with tumor aggressiveness and patient survival rate. Imaging of TAMs and metastasis-associated macrophages could enable early malignancy detection, as well as response assessment of macrophage targeted immunotherapies. In one example, a pH-responsive ⁶⁴Cu-labelled polymeric nanoparticle was used for PET imaging of small occult tumors and outperforms ¹⁸F-FDG in multiple mouse models (Huang. G. et al. Nat Biomed Eng 2019, 4:314-324). In one example, polyglucose nanoparticles labeled with ⁶⁴Cu was used for PET imaging of TAMs in a mouse model (Kim, H. Y. et al., ACS Nano 2018, 12:12015-12029). In another example, dextran nanoparticles labelled with ⁸⁹Zr using a deferoxamine chelator was used for PET imaging of TAMs in a mouse model (Keliher, E. J. et al. Bioconjug Chem 2011, 22: 2383-2389).

Imaging of cancer and its metastasis comprises:
(1) preparing or providing a composition comprising (i) at least one fluorocarbon, (ii) at least one fluorous hydroxamic acid chelator, and (iii) at least one radioisotope;
(2) optionally purifying, diluting, pH adjusting or other treatments of the composition in (1);
(3) administering the composition from (1) or (2) to a subject, wherein administration method can be oral administration, intravenous injection, intramuscular injection, intraarterial injection, subcutaneous injection or others (e.g., one or more administration methods);
(4) imaging of the subject in (3) after administration of the compositions from (1) or (2) to obtain acquired images using imaging methods are selected from the group consisting of PET, SPECT, CT, FMRI, and MRI (e.g., images acquired at one or more times after the administering of compositions of (1) or (2); and
(5) optionally analyzing of acquired images from (4), e.g., with the aid of a computer and software.

The imaging methods can be used for imaging of cancer. For example, images can be acquired at different timepoints following the administering to monitor the development and spread of cancer. In addition, or alternatively, acquired images can be quantified to evaluate the stage and progression of cancer.

The instant disclosure also provides compositions (e.g., compositions comprising (i) at least one fluorocarbon, (ii) at least one fluorous hydroxamic acid chelator, and (iii) at least one radioisotope) and methods for drug delivery for the treatment of diseases such as inflammatory diseases and cancers.

Compounds comprising fluorocarbons have emerged as a versatile platform for drug delivery and cancer therapy. Fluorocarbons have been widely studied as oxygen carriers due to their ability to dissolve large amount of gas. Fluorocarbons are one of the major candidates for blood substitutes. They were also used to alleviate tumor hypoxia and improve the efficiency of cancer radiotherapies, which relies on oxygen supply to generate free radicals to kill cancer cells. Besides oxygen delivery, fluorocarbons have been explored for the delivery of other drugs. In one example, fluorinated polypeptides were prepared for highly efficient delivery of small interfering RNA (siRNA), which was used for the treatment of acute lung injury (Ge, C., et al, Nano Letters 2020, 20:1738-1746). In one example, a fluorocarbon vector-antigen construct was made for the delivery of influenza antigens to immune cells, which is useful as vaccine and immunotherapies (Bonnet, D. et al, U.S. Published Patent Appl. No. 2009/0191233).

The instant disclosure therefore provides a radiotherapy comprising administering a composition comprising (i) at least one fluorocarbon, (ii) at least one fluorous hydroxamic acid chelator, and (iii) at least one radioisotope to a subject, wherein the radioisotope is selected from the group consisting of ⁹⁰Y, ⁶⁰Co, ¹³⁷Cs, ¹⁹²Ir, ²²⁶Ra, ¹⁷⁷Lu, ¹⁵³Sm and others. The radiotherapy can be used for treatment of cancer.

The disclosure also provides a ‘theragnostic’ agent that serves both as a diagnostic agent and therapeutic agent. Theragnostic marks a transition from convention medicine to personalized and precise medicine and has received considerable development. For example, iodine-131 therapy is used for the treatment of thyroid cancer (beta radiation) while enables non-invasive diagnosis with SPECT imaging (gamma radiation). In another example, a combination of positron-emissive Ga-68 and beta-emissive Lu-177 is used for a simultaneous treatment and diagnosis of neuroendocrine Tumors.

The disclosure therefore provides a ‘theragnostic’ agent comprising (i) at least one fluorocarbon, (ii) at least one fluorous hydroxamic acid chelator, and (iii) at least one radioisotope, wherein the radioisotope is selected from the group consisting of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺, ⁸⁶Y³⁺, ⁹⁰Y, ⁶⁰Co, ¹³⁷Cs, ¹⁹²Ir, ²²⁶Ra, ¹⁷⁷Lu, and ¹⁵³Sm combinations thereof. The theragnostic agent can be used for the diagnosis and treatment of diseases, such as inflammation, cancer and others.

The disclosure also provides methods and compositions (e.g., compositions comprising (i) at least one fluorocarbon, (ii) at least one fluorous hydroxamic acid chelator, and (iii) at least one radioisotope) for cell tracking. The cells can be natural cells or engineered cells, such as cells expressing recombinant proteins through gene editing. Cells labelled with compositions comprising (i) at least one fluorocarbon, (ii) at least one fluorous hydroxamic acid chelator, and (iii) at least one radioisotope can be tracked in vivo or ex vivo with imaging techniques.

Cell therapy, in particular T-cell therapy, is revolutionizing personalized medicine and promised to treat many non-curable diseases. A dozen of cell therapies have been approved by U.S. Food & Drug Administration (FDA) for clinic uses, with many others are in clinic trials. The ability to localize and monitor the migration pathways of such therapies will potentially improve their design consideration and facilitate their clinical translation. However, tracking the fate of administered cell therapy is an unmet medical need. In-111 white blood cell (WBC) scan is one of the very few clinically used methods for cell tracking. It works by harvesting white blood cells from patients, label them with radioactive ¹¹¹In-oxine, infusion the cells back into the patients and track cell locations with gamma scan (scintigraphy), which helps the diagnosis of inflammatory diseases, such as osteomyelitis. Optical imaging method using fluorescent dyes, fluorescent proteins or quantum dots have been used in cell tracking but has limited translational potential due to low tissue penetration of visible lights. Iron oxide nanoparticles have been used in preclinical studies and clinic trials for T-cell tracking, using contrasted MR imaging or magnetic particle imaging (e.g., U.S. Pat. No. 9,579,349B2 and Published PCT Appl. No. WO2016/008395A1). Fluorocarbon nanoemulsions have been used for the tracking various types of cells and cell products, such as T-cells, dendritic cells and splenocytes, using FMRI technique (e.g., U.S. Pat. Nos. 8,449,866B2; and 8,263,043B2).

The disclosure therefore provides compositions comprising (i) at least one fluorocarbon, (ii) at least one fluorous hydroxamic acid chelator, and (iii) at least one radioisotope for non-invasive tracking of cells with one or more imaging techniques, such as PET, SPECT and FMRI. The methods for cell tracking can include the steps of:
(1) labeling cells with the compositions described herein (e.g., a composition comprising (i) at least one fluorocarbon, (ii) at least one fluorous hydroxamic acid chelator, and (iii) at least one radioisotope). Proper conditions, such as nutrition, oxygen, pH, temperature, can be supplied to maintain the functionality of cells. Cell labeling time is variable from 0-72 h, in particular from 1-12 h, to allow the association of the said compound with cells.
(2) optionally washing the labelled cells three times with buffer solution. Collect the labelled cells by centrifugation. In some embodiments, the number and viability of cells are measured using proper techniques and reagents, such as microscope, cell counter and others. The association level of the composition with cells can be measured using proper techniques, such as NMR, gamma counter and others.
(3) administering of the labelled cells from (1) or (2) to a subject.
(4) tracking a biodistribution of administered cells in the subject by acquiring images using non-invasive imaging methods, such as PET, SPECT, MRI and FMRI. Images can be acquired at multiple timepoints after administration to assess the migration behavior of the cells. More than one imaging technique can be used to, among other things, improve accuracy. Acquired images can be quantified with suitable software to calculate the number of administered cells in a specific area, such as in the tumor, inflammatory foci, liver, spleen or others.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

### EXAMPLES

The present invention can be better understood by reference to the following examples which are offered by way of illustration. The present invention is not limited to the examples given herein.

### Materials and Methods

Except where noted, chemicals were purchased from Sigma-Aldrich (St. Louis, MO) or TCI America (Portland, OR), and solvents were purchased from Thermo Fisher Scientific (Pittsburgh, PA) and used as received. Fluorous solvents were purchased from Exfluor Research Corporation (Round Rock, TX). Zirconium-89 (4-5 mCi in 1 M HCl or oxalic acid) was purchased from the University of Wisconsin-Madison. All handling and disposal of radioactive materials met local and Federal regulations.

All synthetic reactions were carried out under N₂ unless otherwise noted. Reactions were monitored by thin layer chromatography and LC-MS (Ion Trap XCT with 1100 LC, Agilent, Santa Clara, CA) using an analytical Luna C18(2) reverse-phase column (Phenomenex, Torrance, CA), MeCN/H₂O (with 0.05% v/v CF₃CO₂H) linear gradients, 1 mL/min flow, and ESI positive or negative ion mode. Compounds were purified by pre-packed silica gel columns on a CombiFlash automated system (Lincoln, NE, USA) equipped with a UV-Vis detector.

High-resolution mass spectrometry (HRMS) was performed by the Molecular Mass Spectrometry Facility at the University of California San Diego. UV-Vis absorption spectra were recorded on a Shimadzu UV-2700 (Kyoto, Japan) spectrophotometer. Nanoemulsion size and polydispersity was measured by DLS on a Malvern Zetasizer ZS (Malvern, PA). Solution NMR measurements were performed on a 9.4 Tesla spectrometer (AVANCE III HD-NanoBay, Bruker, Billerica, MA).

Female mice for in vivo experiments were C57BL6 (8-10 weeks) purchased from The Jackson Laboratory (Bar Harbor, ME), as well as Balb/c (6-7 weeks) and CD1 (7-9 weeks) from Envigo (Indianapolis, IN). All animal experiments followed protocols that were approved by University of California San Diego's Institutional Animal Care and Use Committee (IACUC).

### PET Imaging

Positron emission tomography (PET) is a molecular imaging technique that commonly relies on positron-emissive compounds administered to a subject to visualize metabolic activities in a non-invasive manner. Annihilation occurs in the subject when a positron collides with an electron, producing two 511 keV photons at opposite directions that can be recorded by a sophisticated PET camera system comprising rings of detectors made of scintillation crystals (e.g. bismuth germanium oxide) and photomultiplier tubes. PET imaging is often coupled with computed tomography (CT) for anatomical correlation (PET/CT).

PET is a nuclear imaging method that provides very high sensitivity down to picomolar tracer concentration. It is much more sensitive than conventional ultrasound and MRI method. In the past decade, PET scanning has expanded significantly, with around 2 million clinic scans performed in the United States annually. For PET imaging, a compound is labelled with a positron-emissive radioisotope (other name: radionuclide), such as ¹⁸F, which has a decay half-life (t_{1/2}) of around 110 min. The most popular radiotracer used for PET imaging is a ¹⁸F labelled 2-Deoxy-2-fluoroglucose (¹⁸F-FDG). It is an analogue of glucose that monitors glucose metabolism and is widely used in PET diagnosis, especially in oncology. Besides ¹⁸F, other short-lived radioisotopes have been produced for PET imaging, such as ¹¹C (t_{1/2}=20 min), ¹³N (t_{1/2}=10 min), ¹⁵O (t_{1/2}=2 min), ⁶⁸Ga (t_{1/2}=67 min), ⁶⁴Cu (t_{1/2}=12.7 h), ⁸⁹Zr (t_{1/2}=78.4 h), ⁸²Rb (t_{1/2}=1.3 min). While in a few circumstance radioisotopes can be injected directly, such as Na ¹⁸F for imaging of bone disease, in most cases they are incorporated into biocompatible compounds as radiotracers, which then associate with parts of the body to be visualized.

PET imaging is a quantitative imaging method. Quantification can be achieved either in vivo or ex vivo or both. In vivo, one can draw a region-of-interest (ROI) in a subject to determine the standard uptake value (SUV) or percent-of-injected dose (% ID), which can be decay-corrected to a given timepoint. For ex vivo, one can also use gamma counter to measure the dosage level of certain part of a subject, such as spleen or liver, and calculate the SUV or % ID. One can also quantify the uptake level of the radiotracer by cells using a gamma counter.

PET/CT data were acquired using Inveon (Siemens, Malvern, PA, USA) and G8 (SOFIE, Culver City, CA, USA) scanners. Animals were anesthetized using 1-2% isoflurane in oxygen and warmed using and heated pads to maintain body temperature throughout the procedure. PET emission data was acquired for 10 minutes approximately 24 hours after ⁸⁹Zr FERM injection. PET images were reconstructed using an OSEM3D-SPMAP algorithm with scatter and CT-based attenuation corrections. CT images were acquired a 80 kVp tube voltage and 500 μA current, and 220 projections were acquired over a half rotation plus the fan angle. CT images were reconstructed using a Feldkamp algorithm with a Shepp-Logan filter, mouse beam hardening correction and slight noise reduction.

### MRI

Conventional MRI detects the density and relaxation properties of hydrogen atoms (¹H) in molecules contained in a subject placed in an external magnetic field produced by superconducting magnets. Any atoms (e.g. ¹H, ³He, ¹¹B, ¹³C, ¹⁵N, ¹⁷O, ¹⁹F, ²³Na, ³¹P, ³³S) that contain an odd number of protons and neutrons can be potentially detected through the magnetic resonance effect. However, ¹H is the sole atom that is routinely used in clinic MRI, due to the abundance of water and lipids in the body and a limited sensitivity of MRI that requires at least a micromolar concentration for atoms to detected. While ¹H MRI is widely used in medical diagnosis, there are many cases in which it is insufficient in detecting the pathological sites. Different from conventional MRI that detects ¹H, FMRI detects of ¹⁹F administered to a subject. There are several benefits of FMRI. There is only a very small amount ¹⁹F atoms in the body that are mostly immobilized in the bone, far below the detection limit of a regular MRI scanner, thus allowing a clear ‘hot-spot’ imaging without background noise. Among those MRI-active nuclei, ¹⁹F has several intrinsic advantages, such as a natural abundance of 100%, a gyromagnetic ratio 94% of ¹H, a magnetic sensitivity 83% of ¹H and a broad range of chemical shift (˜400 ppm vs ˜20 ppm for ¹H) that allows multi-color imaging.

Both ¹H and ¹⁹F MRI share the same physical principle, from radiofrequency radiation, signal receiving to spatial resolution and imaging reconstruction. Therefore, only a few modifications on hardware and software are required to adapt clinic MRI scanners for ¹⁹F acquisition. For anatomical reference, a fast ¹H scan is usually performed before or after a ¹⁹F scan, by using ¹⁹F/¹H dual-tuned radiofrequency coil. A merged ¹H/¹⁹F shows the exact location of fluorine atoms in the field of view. Advancement in hardware and software allows the acquisition of both ¹H and ¹⁹F simultaneously, with the use of suitable radiofrequency coil, to shorten scanning time. In the past decade, FMRI has gained considerable progress in preclinical and clinic studies. In one example, FMRI was successfully performed on human lungs at 0.5 Tesla MR scanner using fluorocarbon (Pavlova et al, Magn Reson Med 2020, 00:1-7).

FMRI is a highly quantitative imaging method. The absolute number of fluorine atoms in a region-of-interest (ROI) can be calculated based on the signal-to-noise ratio between the ROI and an external reference with known fluorine concentration. In some embodiment, a tube comprising a known concentration of ¹⁹F was placed alongside the subject during FMRI scanning for quantification purpose. One can also calculate the number of fluorine atoms per cell ex vivo using 1D NMR spectroscopy. The quantification is comparable between different subjects. For example, one can compare the number of fluorine atoms in the ROI, such as a tumor, among different subjects. In some embodiment, the number of fluorine atoms present in a ROI is used to evaluate the severity or stage of a disease. In some embodiments, the number of fluorine atoms present in a cell is used to determine the optimal dosage, formulation recipe or incubation conditions or others.

Many fluorocarbons have been used for FMRI. In some embodiments, the compound comprising fluorocarbon is selected from the group consisting of perfluorooctyl bromide (PFOB), perfluoro-15-crown-5-ether (PFCE), perfluoropolyethers (PFPE), perfluorotrialkylamine (PFTA), perfluorodecalin (PFD), perfluorohexane (PFH), perfluorononane (PFN), hexafluorobenzene (HFB), PERFECTA and others (e.g., perfluoro-tert-butyl-cyclohexane (PFTBC)).

MRI scanning was performed using a Bruker BioSpec 11.7 T MRI system running ParaVision 6 software and a dual-tuned ¹⁹F/¹H 38 mm volume coil. Mice were anesthetized using 1-2% isoflurane in oxygen, and body temperature was maintained throughout the procedure using a heated air system. ¹⁹F images were acquired using a RARE (rapid acquisition with relaxation enhancement) sequence with repetition time (TR) 1 s, echo time (TE) 20 ms, RARE factor 8, 30×45 mm field of view, matrix size of 32×48, 2 mm thick coronal slices (12), 150 averages and 15 min scan time. Anatomical ¹H scans were acquired using a RARE sequence with TR=2 s, TE=20.5 ms, RARE factor=8, 10 averages, 1 min scan time, 1 mm thick slices (24), and field of view and orientation identical to the ¹⁹F images.

### Simultaneous PET/FMRI

Multimodal imaging, where more than one imaging techniques were involved, has demonstrated great benefits in diagnosis and treatment. PET/CT, a dual imaging method, has been established for decades in many clinic conditions, such as oncology. Compared with PET/CT, PET/MRI reduces the radiation exposure to patients by ˜50%, has higher resolution that can detects smaller lesions that are easily missed in PET/CT. In the past, a major challenging in integrating PET and MRI is that the detectors of PET are deeply interrupted by the strong magnetic field of MRI. In the past years, this issue has been largely addressed by innovations in detector materials and scanner configurations. Hybrid PET and ¹H-only MRI scanners are now available in some clinical service, for example, in cardiology, oncology and neurology. PET/MRI have been commercialized by multiple companies (e.g. Siemens, Philips, Bruker, General Electric) for clinic and preclinic studies and proven valuable in clinic diagnosis. Such advances in scanner technologies have advantages of accelerated data acquisition, improved image registration, and motion correction of voluntary or involuntary movement like respiration or bowel peristalsis.

Progress in PET/MRI scanner will likely make it possible to acquire PET/FMRI data, with suitable software and hardware modification. A major hurdle in the clinic translation of FMRI is its low sensitivity, due to the limited amount of fluorocarbons that can be administered. The low-sensitivity of ¹⁹F MRI rendered it prone to false negative diagnosis, whereas the high-sensitivity of PET imaging is prone to false positives, thus a bimodal PET/FMRI readout provides complementary information with high accuracy.

### Single-Photon Emission Computed Tomography (SPECT)

In some embodiments, the present invention provides a method for producing a radiotracer for SPECT imaging. Similar to PET, SPECT is another nuclear imaging method but detects gamma rays with a rotating gamma camera system, which was reconstructed with a computing system to generate 3-dimensional images. The mostly commonly used gamma-emissive radioisotopes for SPECT are ^{99m}Tc⁴⁺, ¹¹¹In³⁺, ^{123,125,131}I. While SPECT has lower resolution and sensitivity, it is less costly and more widely available than PET imaging. In clinic, SPECT scanning is usually applied to evaluate brain disorder and heart diseases, such as dementia, clogged arteries and reduced ejection fraction. While in a few circumstances the ionic radioisotope can be used directly, most of the time the radioisotope needs to be attached to a molecular with specific functions to create a radiotracer that binds to certain parts of the subject. SPECT detects the biodistribution of such radiotracer in the subject in a non-invasive manner.

### Image Quantification and Visualization

Image quantification was performed using VivoQuant software (Invicro, Boston, MA). PET data were calibrated using a 30 mL (Inveon) or 6 mL (G8) phantom. Footpad signals were quantified by placing a cylindrical ROI over the paws and integrating signals. For analysis of IBD models, bone and bone marrow signals were segmented by thresholding CT images. Coarse ROIs were placed over the liver and spleen and segmented by thresholding the PET signal from 1×10⁻⁸ to 2×10⁻⁷ μCi. A single ROI was then placed over the peritoneum for subsequent analysis. ¹⁹F data were quantified from a phantom placed in the field of view. For each image, three ROIs each were placed in the phantom and background to determine values and errors for the phantom and background signals. Histograms were produced using VivoQuant software, with ranges set from 0-1×10⁻⁴% ID per voxel for PET imaging and from 0 to 3×10²⁴ F-atoms/mL for FMRI images. For clarity, beds and phantoms were masked by generating an ROI around the mouse by thresholding the CT image, then exporting as a separate image. For display purposes, PET and FMRI images were co-registered by applying a transformation derived from co-registration of CT and ¹H images using an affine or nonlinear transformation algorithm in VivoQuant, and images were rendered in pseudo-color

### Histology

In the carrageenan acute inflammation model, we employed a dual-mode fluorocarbon nanoemulsion containing a red-fluorescent dye (VS-1000H DM Red, 150 nm droplet size, Celsense, Pittsburgh, PA). The nanoemulsion was injected through tail vein to mice bearing inflammation on the right-paw (n=3). After 24 h, mice were sacrificed and paws were harvested, weighted, cryo-freezed in optimal cutting compound and kept frozen at −80° C. The sections were cut along the palm direction at 10 μm thickness in a cryotome and fixed with 4% paraformaldehyde (PFA), followed by permeabilization and Fc blocking. Slices were stained with rabbit anti-mannose receptor (ab64693, Abcam, Cambridge, UK) as primary antibody and Alexa Fluor 488 goat anti-rabbit (A11008, Thermo Fisher) as the secondary antibody. Nuclei were stained using Hoechst 33342 (62249, Thermo Fisher). Confocal images (CTR 6500, Leica Microsystems, Buffalo Grove, IL) were acquired of sections at 63× magnification. Colon tissues in Swiss rolls were fixed in 2% PFA in PBS and analyzed using histology. Segments were embedded in paraffin, cut into 5 μm-thick transverse sections, and stained with hematoxylin and eosin (H&E) and imaged (200×).

### Statistics

Two-sample unpaired t-test was used for significance analysis. A p<0.05 was considered significant. Statistical software R (http://www.r-project.org) was used for blood sample pharmacokinetic analyses. The function “biexp” was used to build the biexponential model for estimation of two-phase half-lives for both gamma counting and ¹⁹F NMR, and bootstrapping techniques were used to calculate 95% confidence intervals.

### Example 1: Synthesis of FHOA

The starting material FA was prepared in ˜5 g scale as colorless oil by reaction of acryloyl chloride with fluorous amine based on published methods (20) and confirmed by LC-MS (10-100% CH₃CN/H₂O for 20 min then 100% CH₃CN for 5 min, t=23.4 min, [M+H]⁺ measured 522.9, calculated 523.1) and NMR. ¹H NMR (400 MHz, CDCl₃) δ 7.43-7.36 (m, 5H), 6.70 (dd, J=17.1, 10.4 Hz, 1H), 6.44 (dd, J=17.2, 1.9 Hz, 1H), 5.77 (dd, J=10.3, 1.9 Hz, 1H), 4.86 (s, 2H), 3.92 (t, J=8.0 Hz, 2H), 2.44 (tt, J=17.9, 7.7 Hz, 2H). ¹⁹F NMR (376 MHz, CDCl₃) δ −80.77 (t, J=10.62 Hz, 3F), −113.98-−114.40 (m, 2F), −121.88 (s, 2F), −122.85 (s, 2F), −123.05-−123.91 (m, 2F), −126.00-−126.29 (m, 2F).

A mixture of compound FA (4.88 mmol, 2.55 g) and 1,4-diaminobutane (1.08 mmol, 108.8 μL, Sigma-Aldrich) in acetonitrile (50 mL) were refluxed under N₂ for 72 hours to obtain the benzoyl-protected precursor FHOA-OBn. LC-MS showed FHOA-OBn to be the major product (10-100% MeCN/H₂O for 20 min then 100% CH₃CN for 5 min, t=23.7 min, [M+H]⁺ measured 2179.8, calculated 2180.4). After the reaction cooled to room temperature, the solvent was evaporated, and the residue was re-dissolved in ethyl acetate (3 mL). The crude product was purified using a silica gel column (40 g) and eluted with CH₃OH/CH₂Cl₂ (0-8%, 15 min, linear gradient). A major peak centered at 11.5 min was collected and identified as FHOA-OBn by LC-MS. Solvents were evaporated, leaving FHOA-OBn as a colorless oil with a yield of 61±6% (1.44 g, n=3). HRMS (m/z, ESI-TOF) for [M+2H]²⁺ was calculated to be 1091.2207 and 1091.2198 was measured. ¹H NMR (400 MHz, CDCl₃) δ 7.37 (s, 20H, —C₆H₅), 4.82 (s, —OCH₂Ph, 8H), 3.82 (t, J=7.6 Hz, 8H), 2.73 (t, J=7.3 Hz, 8H), 2.52 (t, J=7.1 Hz, 8H), 2.47-2.19 (m, 12H), 1.74 (s, 4H). ¹⁹F NMR (376 MHz, CDCl₃) δ −80.83 (t, J=8.99 Hz, —CF₃, 12F), −114.30 (p, J=18.4 Hz, —CH₂CF₂—, 8F), −121.97 (q, J=14.2 Hz, 8F), −122.95 (s, 8F), −123.50 (t, J=13.8 Hz, 8F), −126.10-−126.30 (m, 8F). ¹³C NMR (101 MHz, CDCl₃) δ 174.71 (C═O), 134.16, 129.37, 129.23, 128.84, 127.15-99.89 (m, —CF₂—, weak), 54.03, 48.38, 38.48, 29.97, 29.73, 27.87 (t, —CH₂CF₂—, J=22.03 Hz), 24.96.

The FHOA-OBn (1.44 g) obtained was dissolved in ethanol (60 mL), then Pd/C (10%, 120 mg) was added. The mixture was stirred at room temperature under 1 atm H₂ for 16 h. LC-MS confirmed the disappearance of FHOA-OBn and formation of FHOA (10-100% CH₃CN/H₂O for 20 min then 100% CH₃CN for 5 min, t=22.6 min, [M+H]⁺ measured 1819.5, calculated 1820.2). The mixture was first filtered through Celite, then through a 0.22 μm membrane to remove insoluble components. Residual solvents were removed by evaporation under vacuum, leaving FHOA as yellow semisolid with a yield of 89±4% (1.06 g, n=3). HRMS (m/z, ESI-TOF) for [M+2H]²⁺ was calculated to be 911.1268 and 911.1267 was measured. ¹H NMR (400 MHz, CDCl₃) δ 3.93 (t, J=8.38 Hz, 8H), 2.93-2.61 (m, 12H), 2.56-2.30 (m, 20H). ¹⁹F NMR (376 MHz, CDCl₃) δ −80.81-81.56 (m, —CF₃, 12F), −114.40-−114.86 (m, —CH₂CF₂—, 8F), −122.11-−122.91 (m, 8F), −122.91-−123.35 (m, 8F), −123.38-−123.95 (m, 8F), −125.52-−127.00 (m, 8F). ¹³C NMR (101 MHz, CDCl₃) δ 173.26 (C═O), 124.12-100.03 (m, —CF₂—, weak), 53.32, 49.90, 40.48, 29.73, 27.97 (m, —CH₂CF₂—), 24.27.

### Example 2: Synthesis of FDFO

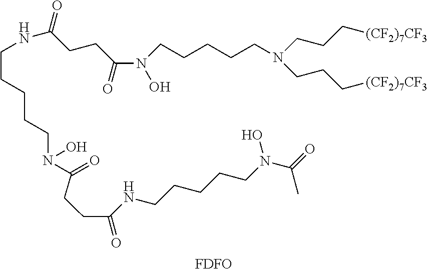

To a round-bottom flask was added deferoxamine mesylate salt (357 mg, 0.5 mmol, Sigma-Aldrich) and 3-(perfluorooctyl) propyl iodide (882 mg, 1.5 mmol, Sigma-Aldrich), followed by dimethylformamide (DMF, 20 mL) and N, N-diisopropylethylamine (0.35 mL, 2 mmol, Sigma-Aldrich). The mixture was heated at 110° C. for 16 h under nitrogen, then cooled to room temperature. The mixture was poured into ice water (100 mL) in a separatory funnel and extract 5 times with hexane/ethyl acetate (10/10 mL). The combined organic extract was washed with saturated NaCl solution (100 mL). The organic extract was dried with anhydrous Na₂SO₄, decanted into a flask, and evaporated under vacuum. The residual was re-suspended in CH₃CN (10 mL), bath sonicated for 5 mm and centrifuged to remove insoluble components. The solution was concentrated to ˜2 mL and loaded onto semi-preparative HPLC for ˜5 times for purification. The product peak was collected and confirmed by LC-MS The combined product was lyophilized and stored at −20° C. for use. Identity of FDFO was further confirmed by fluorine-19 NMR spectroscopy, as well as iron-binding complex (Fe-FDFO) on LC-MS.

### Example 3: Nanoemulsion 1 Preparation and Characterization

A lipid film was prepared by dissolving 115.5 mg egg lecithin (60%, Alfa Aesar, Haverhill, MA), 9.3 mg cholesterol (Anatrace, Maumee, OH) and 16.8 mg DSPE-mPEG(2000) (Avanti Polar Lipids, Alabaster, AL) in chloroform (1.5 mL), followed by rotatory evaporation under N₂ flow, and drying under high vacuum for 24 h. The lipid film was hydrated with purified water (4.8 mL), vortexed at high for 2 min and probe sonicated for 2 mins (Omni Ruptor 250 W, 30% power, Omni International, Tusla, OK). FHOA (1 mM, 2.2 mg) was fully dissolved in PFOB or PFCE (1.2 mL) oil and was added, and the final mixture was vortexed and sonicated sequentially for 2 min each. The pre-emulsion obtained was passed five times through a microfluidizer (LV1, Microfluidics, Newton, MA) operating at 20,000 psi and filtered through a 0.8/0.2 μm Supor membrane (Port Washington, NY) into sterile glass vials. The vials were sealed and stored at 4° C. before use. Nanoemulsion containing a higher concentration of FHOA (10 mM, 22 mg) was prepared in the same way to study the binding behavior by NMR and UV-Vis. Blank PFOB nanoemulsion was formulated in a similar way, except that no FHOA was added. FERM nanoemulsion using a polymeric surfactant (Pluronic F68) was prepared by mixing an aqueous solution of Pluronic F68 (4.8 mL, 31 g/L) with PFCE (1 mM FHOA, 1.2 mL) directly, followed by sonication and microfluidization as stated above. No phase separation was observed for >2 months of storage at 4° C. for all nanoemulsions prepared.

### Example 4: Nanoemulsion 2 Preparation and Characterization

To 4.8 mL fresh-made aqueous solution of Pluronic F68 (31 g/L) was added 1.2 mL of PFCE containing FHOA (2.2 mg, 1 mM). The mixture vortexed at high for 2 min and probe sonicated for 2 mins (Omni Ruptor 250 W, 30% power, Omni International, Tusla, OK). The pre-emulsion obtained was passed five times through a microfluidizer (LV1, Microfluidics, Newton, MA) operating at 20,000 psi and filtered through a 0.8/0.2 μm Supor membrane (Port Washington, NY) into sterile glass vials. The vials were sealed and stored at 4° C. before use. Nanoemulsion containing a higher concentration of FHOA (10 mM, 22 mg) was prepared in the same way. Blank PFCE nanoemulsion was formulated in a similar way, except that no FHOA was added. Emulsions using other polymeric surfactants, such as Pluronic F127, Tween 20 or Zonyl FS-300, can be prepared in the same way. No phase separation was observed for >2 months of storage at 4° C. for all nanoemulsions prepared.

### Example 5: Nanoemulsion 3 Preparation and Characterization

A lipid film was prepared by dissolving FDFO (2.2 mg), 115.5 mg egg lecithin (60%, Alfa Aesar, Haverhill, MA), 9.3 mg cholesterol (Anatrace, Maumee, OH), 16.8 mg DSPE-mPEG(2000) (Avanti Polar Lipids, Alabaster, AL) in chloroform (1.5 mL), followed by rotatory evaporation under N₂ flow, and drying under high vacuum for 24 h. The lipid film prepared was hydrated with purified water (4.8 mL), vortexed at high for 2 min and probe sonicated for 2 mins (Omni Ruptor 250 W, 30% power, Omni International, Tusla, OK). PFOB or PFCE (1.2 mL) was added to the lipids, and the final mixture was vortexed and sonicated sequentially for 2 min each. The pre-emulsion obtained was passed five times through a microfluidizer (LV1, Microfluidics, Newton, MA) operating at 20,000 psi and filtered through a 0.8/0.2 μm Supor membrane (Port Washington, NY) into sterile glass vials. The vials were sealed and stored at 4° C. before use. Nanoemulsion containing a higher concentration of DFDO (22 mg) was prepared in the same way. Blank nanoemulsion was formulated in a similar way, except that no FDFO was used. No phase separation was observed for >2 months of storage at 4° C. for all nanoemulsions prepared.

### Example 6: Nanoemulsion 4 Preparation and Characterization

A lipid film was prepared by dissolving 314 mg of (EYP), 63 mg Cholesterol, 7 mg dipalmitoyl-sn-glycero-3-phosphoserine (DPPS) in a small volume of chloroform and drying in a glass vial under argon while rotating. Lipids were rehydrated by addition of 7.05 ml of water with 204 mg Mannitol and vortexing for 1 minute, followed by sonication for 2 minutes. The fluorous phase was prepared by adding 564 mg of FDK, 204 mg 1-(perfluoro-n-hexyl)decane and 4.896 g of PFOB to a glass vial and vortexing. The fluorous phase was then added to the lipid suspension and vortexed, followed sonication for 2 minutes. The fine emulsion was prepared by five passes through a microfluidizer (LV1, Microfluidics, Newton, MA) operating at 20,000 psi, followed by filtration into a sterile glass vial. Following a modest initial increase in size, no appreciable change was observed for two months after emulsion preparation, and no phase separation was observed.

### Example 7: Nanoemulsion 5 Preparation and Characterization 5

1.125 g of PFPE or FDK was added to 625 μl of a 100 mg/ml solution of pluronic surfactants in a 10 ml glass vial and vortexed for 10 seconds. 5.45 ml of water was added to the vial and the mixture vortexed again for 10 seconds followed by sonication for 2 minutes and 4 passes through a microfluidizer (LV1, Microfluidics, Newton, MA) operating at 20,000 psi. The emulsion was filtered into a sterile glass vial.

### Example 8: Buffering ⁸⁹ZrCl₄ Radiolabeled Emulsion with 1 M HCl

The concentration of ⁸⁹ZrCl₄ is assumed to be negligible in comparison to that of the HCl. The ratio of bulk perfluorocarbon to water prior to emulsification is known from the mass of perfluorocarbon and the volume of water added to the emulsion. Since the concentration of emulsion is routinely determined from ¹⁹F NMR after the final emulsion is formulated, the fraction of water in the final emulsion can be calculated from a dilution factor determined from concentrations of the perfluorocarbon. It is assumed that the lipid and perfluorocarbon components do not contribute to the osmolality, and therefore only the acqueous components are included in the osmolality calculations.

If 1 M Tris is used as a buffer, the volume to be added in μl is determined from the Henderson-Hasselbalch equation and the volume of 1 M Tris added in μl per ml of emulsion is as so: (Vol of ZrCl₄ added in μl)×10^{-0.27}/(aqueous volume of emulsion in ml)

To adjust the osmolality for administration (to 280 mOsm/kg): Osmolality=(vol of Tris added in μl/ml)+2×(vol. of ZrCl4 added in μl/ml). The osmolality gap is: 280—calculated osmolality. Thus, if propylene glycol is used as the osmolyte, assuming an osmolality of 132 mOsm/kg: Volume of propylene glycol to add=(osmolality gap/132)×(aqueous volume in ml/100).

### Example 9: Cell Tracking

In one example, fluorocarbon emulsion 2 (0.9 mL) prepared above was radiolabeled with 1 mCi ⁸⁹Zr by a sample mixing for 3 h, followed by purification with a prepacked gel filtration column (NAP-10, GE Healthcare). Splenocytes were isolated from mice spleen by placing it into a cell strainer and mashed into the petri dish. Cells were transferred to a conical and centrifuged for 5 min at 250 RCF. The supernatant was discarded. Cells were resuspended in 1 mL ACK lysis buffer, incubated at room temperature for 10 min, and centrifuged again in 10 mL DMEM buffer for 5 min. The supernatant was discarded, and cell pellet was resuspended in 5 mL DMEM buffer. Cell number was counted using trypan blue on an automated cell counter. A total of 60 million Cells were diluted in cell culture dishes at 2 million per mL of DMEM buffer. The ⁸⁹Zr labelled emulsion was added to splenocytes in cell culture tubes at 40 μCi ⁸⁹Zr per mL. Cells were cultured in an incubator at 37 degree with 5% CO₂ for 4 h. Cells were centrifuged for 5 min at 250 RCF, and the supernatant was discarded. The cell pellets were washed 3 times with 1×PBS buffer to remove unlabeled nanoemulsion. The labelled cells were suspended in 1×PBS at a concentration of 10 million cells per 0.2 mL, and then injected into mice (n=3) through tail vein. Mice were scanned on a small animal PET/CT scanner at various time points up to 5 days post injection to visualize the migration pathway of splenocytes in vivo.

### Example 10: Metal Binding

Direct binding of ZrCl₄ to FHOA in solution was characterized by NMR spectroscopy. 0-2 equivalents of 40 mM ZrCl₄ in CD₃OD were titrated into 0.4 mL of 0.2 mM FHOA in CD₃OD in 5 mm NMR tubes. Samples were incubated at room temperature for 1 hour prior to acquisition of ¹H and ¹⁹F NMR spectra. To determine binding kinetics, ¹⁹F NMR spectra were recorded at 10, 20 and 30 min timepoints after addition of 1 equivalent of ZrCl₄.

Binding and transmetallation of Zr⁴⁺ and Fe³⁺ to FHOA was observed by UV-vis spectroscopy and ¹⁹F T₁ relaxation. 0.16 μmol of FeCl₃ or ZrCl₄ in 90% H₂O/10% D₂O was added to 40 μL of FERM nanoemulsion in 260 □L of H₂O/10% D₂O. After incubation for 3 hours, ¹⁹F T₁ relaxation times were determined using inversion recovery experiments, and UV-vis spectra recorded. To investigate transmetallation, an equal amount of ZrCl₄ or FeCl₃ was added to Fe and Zr bound emulsions, respectively, and T₁ and UV-vis experiments were repeated 16 h after addition. To determine transmetallation rate, UV-Vis spectra were also recorded every 5 min automatically in the 16 h period following addition of ZrCl₄ to Fe-saturated nanoemulsion.

### Example 11: Radiolabeling with ⁸⁹Zr

For radiolabeling, 0.8 mCi of ⁸⁹ZrCl₄ (5 μL in 1M HCl) was added to 1 mL of FERM nanoemulsion and gently mixed in a vial. After incubation at room temperature for 3 h, the mixture was desalted into 10 mM Tris-HCl buffer (pH=8.0) containing 2% (v/v) of propylene glycol using a Sephadex G-25 gel filtration column (NAP-10, GE Healthcare, Chicago, IL) and collected in a glass vial. The nanoemulsion was typically adjusted to a concentration of 100 μCi per 0.2 mL with buffer for in vivo injection. The radiolabeling yield of FERM was calculated from the ratio of activity in the collected eluate to total activity. A prolonged reaction time (16 h) did not significantly increase the yield. Quantitative ¹⁹F NMR from decayed samples showed a typical recovery of 61.2±8.0% (0.8 mL, n=3) of total fluorine atoms loaded into the column.

For radiolabeling with ⁸⁹Zr-oxlate (in 1 M oxalic acid), 0.6 mCi (˜10 μL) of ⁸⁹Zr-oxlate in 30 μL 1 M NaHCO₃, and 10 μL 1 M Tris buffer (pH=8.0) was added to 1 mL of lipid FERM nanoemulsion, which resulted in a final pH between 6-6.5 as tested by pH paper. After incubation at room temperature for 3 h, the mixture was desalted using the above method.

### Example 12: MTT Assay

The murine macrophage cell line RAW264.7 (ATCC, Manassas, VA) was maintained in DMEM containing 10% fetal bovine serum (FBS), 10 mM HEPES, 1 mM sodium pyruvate, and 1.5 g/L sodium bicarbonate at 37° C. in 5% CO₂ atmosphere. Cells were grown in 10 mL cell culture tubes. Nanoemulsion with FHOA (1 mM in oil) or without chelate were added at a fluorine concentration of 5 mg/mL overnight. Cells without nanoemulsion labeling were used as control groups. After incubation overnight at 37° C., cells were washed three times in PBS and counted using a Countess II FL Automated Cell Counter (Fisher Scientific, Waltham, WA). The labeled and unlabeled cells were seeded in 96-well plate with 10⁵/well in cell culture medium. An MTT assay kit (ab211091, Abcam, Cambridge, UK) was used following the manufacturer's protocol at 0, 18, 24, and 48 hours. The absorbance at OD=590 nm was measured using an Infinite 200 plate reader (Tecan, Mannedorf, Switzerland).

### Example 13: Blood Circulation Time and Agent Stability In Vivo

The blood half-life of FERM nanoemulsion (PFCE) was monitored using γ-counting and ¹⁹F NMR. The ⁸⁹Zr labeled FERM was injected into Group 1 (n=3) and Group 2 (n=3) mice through the tail vein at an activity dose of 85 μCi (0.2 mL). Mean weights and standard deviations (±) of mice in Groups 1 and 2 were 41.4±0.9 g and 41.4±1 g, with no significant difference (p>0.05) between the two groups. Mice were anesthetized using 1-2% isoflurane in oxygen, and blood samples were collected from retro-orbital sinus using capillaries. Blood was drawn at 5, 15, 60, 240, 480 min post injection (Group 1) and 10, 30, 120, 360, 1440 min post injection (Group 2). A 100 μL blood sample was pipetted into to a 5 mm NMR tube, followed by the addition of lysis buffer (100 μL). The radioactivity of each sample was assayed and decay-corrected to the injection time. The samples were stored at 4° C. for a 5-week ⁸⁹Zr decay period, and a solution of sodium trifluoroacetate (NaTFA, 25 mM) in D₂O (50 μL) was added as internal reference. ¹⁹F NMR spectra were acquired using the standard Bruker sequence with repetition time 10 s, number of averages 32 and 32,768 points. The ratio of integrals of the PFCE peak at −91.8 ppm and NaTFA reference at −75.4 ppm were used to calculate the fluorine content in the blood sample. A bi-exponential decay model was used to calculate blood circulation times.

### Example 14: Carrageenan Acute Inflammation Model

λ-Carrageenan plant mucopolysaccharide (Sigma-Aldrich) at 50 μL dose (2% in saline) was injected into the right paw of female CD1 mice (7-9 weeks old). Swelling of the paw was confirmed visually and by measurement of paw width and thickness by calipers. Mice were anesthetized, FERM nanoemulsion was injected (100 to 1 μCi, 0.2 mL) through tail vein, and ¹⁹F/¹H MRI, PET and CT images were acquired 24 h post-injection.

### Example 15: Inflammatory IBD Model

IBD was induced in female, 8-10 week C57BL/6 mice (n=9) by administration of 3% dextran sulfate sodium salt (DSS) via drinking water ad libitum for 7 days prior to injection of imaging agents. Control mice (n=3) received normal drinking water. Disease progression was monitored daily by body weight loss, stool score and hemoccult score (FIG. S4). ⁸⁹Zr labeled FERM nanoemulsion (100 μCi, 0.2 mL) was injected through tail vein, and ¹⁹F/¹H MRI, PET and CT images were acquired 24 h post-injection.

### Example 16: 4T1 Tumor Model

Luciferase-expressing 4T1-luc2 cells (CRL-2539-luc2, ATCC, Manassas, VA) were maintained in RPMI-1640 medium containing 10% FBS and 8 ug/ml Blasticidin. Cells (5×10⁶) were suspended in 50 μl PBS containing 50% matrigel and inoculated into the fourth mammary fat pad of 6-7 week female Balb/c mice. Tumor volumes were measured twice weekly by calipers, and mice were sorted into two groups (n=5 each) when tumors reached a volume of 200-350 mm³ (2-week cohort) and 900-1200 mm³ (5-week cohort). The ⁸⁹Zr labeled FERM nanoemulsion was injected into mice via tail vein at a dose of 100 μCi (200 μL), and ¹⁹F/¹H MRI, PET, CT and BLI images were acquired 24 h post-injection. Mice were sacrificed after imaging, lungs and tumors were harvested, and dosimetry measurements were performed on the tissues.

The disclosure provides for the following example embodiments, the numbering of which is not to be construed as designating levels of importance:

Embodiment 1 relates to a composition comprising:
at least one fluorocarbon; and
at least one fluorous hydroxamic acid chelator,
wherein the at least one fluorous hydroxamic acid chelator is at least partially soluble in the fluorocarbon.

Embodiment 2 relates to the composition of Embodiment 1, wherein the at least one fluorocarbon forms a fluorous phase and the composition is a fluorous phase-encapsulated at least one fluorous hydroxamic acid chelator.

Embodiment 3 relates to the composition of Embodiment 1, wherein the composition is an emulsion.

Embodiment 4 relates to the composition of Embodiment 2, wherein the emulsion forms droplets having a diameter from about 5 nm to about 500 nm as determined by dynamic light scattering (DLS).

Embodiment 5 relates to the composition of Embodiments 1-4, further comprising (iii) at least one radioisotope chelated by the at least one fluorous hydroxamic acid chelator.

Embodiment 6 relates to the composition of Embodiment 5, wherein the at least one radioisotope is at least one of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺, and ⁸⁶Y³⁺.

Embodiment 7 relates to the composition of Embodiment 5 or 6, wherein the at least one radioisotope is ⁸⁹Zr⁴⁺.

Embodiment 8 relates to the composition of Embodiments 1-7, wherein the at least one fluorocarbon is at least one of:

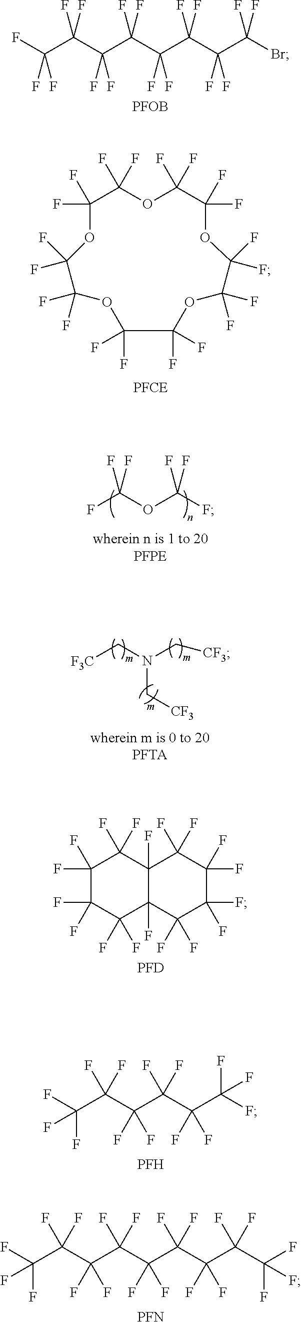

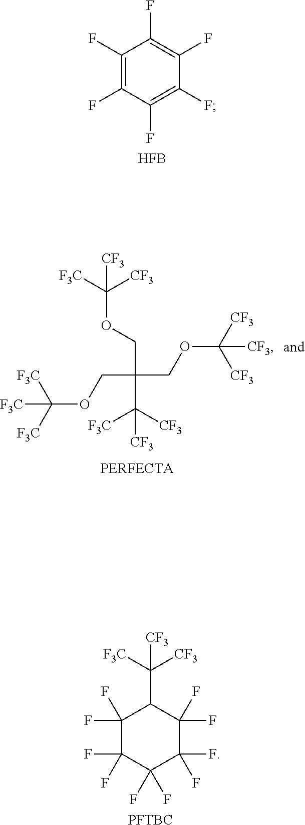

Embodiment 9 relates to the composition of Embodiments 1-8, wherein the at least one fluorous hydroxamic acid chelator is at least one of:

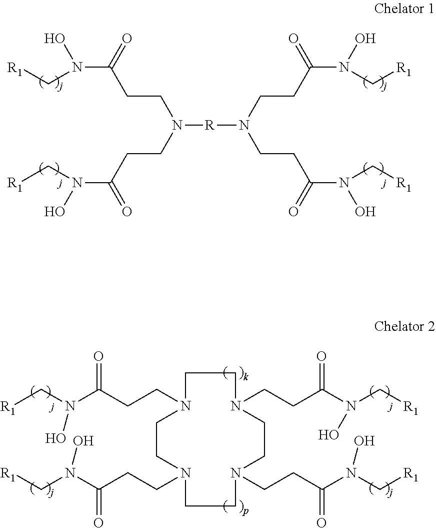

wherein R is a linker; each j is independently 1 to 20; k and p are each independently 0 to 3; each R₁ is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein each p and q is independently 0 to 20;

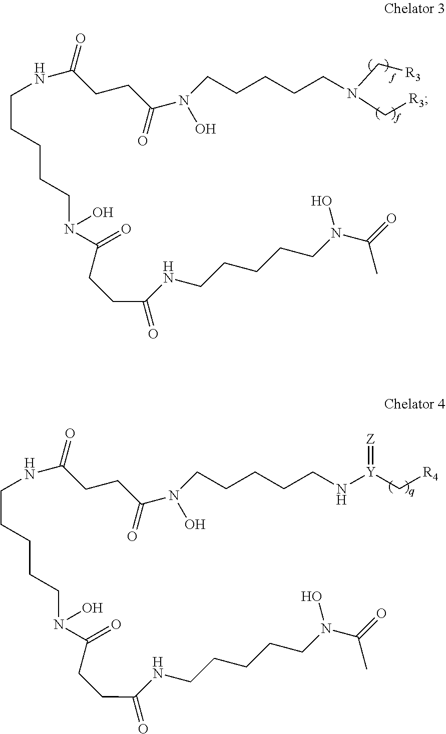

wherein each f and q is independently from 0 to 20; Y and Z are each selected from the group consisting of C, N, O, Si, P, and S; each Rₐ and R₄ is independently selected from the group consisting of —(CF₂)ₙ—CF₃, —O—(CF₂)ₙ—CF₃, —O—CF₂—(OCF₂CF₂)ₙ—OCF₃ and —[(CH₂)ₘ(CF₂)ₙCF₃]₂, wherein p and q are 0 to 20; and

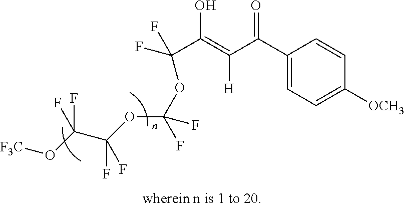

Embodiment 10 relates to the composition of Embodiment 9, wherein Y and N form the groups C═O, C═S or P═O.

Embodiment 11 relates to the composition of Embodiments 1-10, wherein the at least one fluorous hydroxamic acid chelator is at least one of:

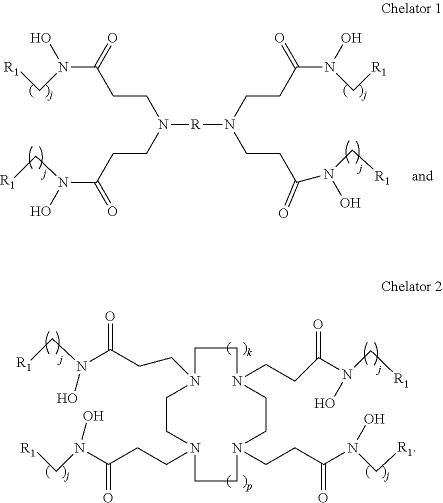

Embodiment 12 relates to the composition of Embodiments 9-11, wherein R is a C₃-C₆-alkyl linker.

Embodiment 13 relates to the composition of Embodiments 1-12, wherein the at least one fluorous hydroxamic acid chelator is at least one of:

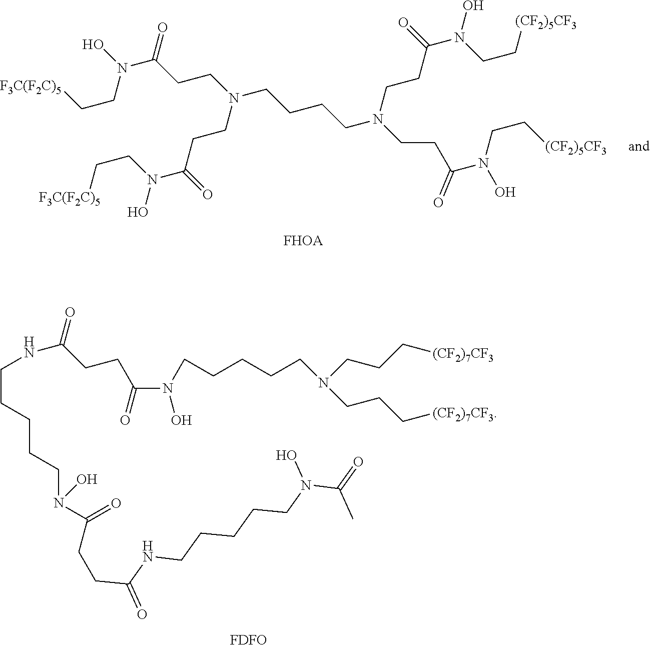

Embodiment 14 relates to the composition of Embodiments 1-13 further comprising at least one surfactant.

Embodiment 15 relates to the composition of Embodiment 14, wherein the at least one surfactant is at least one of egg lecithin, soybean lecithin, sunflower oil, 1,2-distearoyl-sn-glycero-3-phosphorylethanolamine (DSPE), DSPE-PEG2000, 1,2-distearoyl-sn-glycero-3-phosphoethanolamine-N-[methoxy(polyethylene glycol)-2000, DSPE-PEG3000, DSPE-PEG5000, DPPE, DPPE-PEG, cholesterol, Kolliphor EL, mannitol, CH₃—(CH₂)₅—(CF₂)₅—CF₃, Zonyl FS-300, Pluronic F68, Pluronic F127, Tween 20, and Tween 80.

Embodiment 16 relates to the composition of Embodiment 14 or 15, wherein the at least one surfactant is at least one of egg lecithin, DSPE-PEG2000, and cholesterol.

Embodiment 17 relates to a method of making a composition comprising:
at least one fluorocarbon; and
at least one fluorous hydroxamic acid chelator,
the method comprising contacting the at least one fluorous hydroxamic acid chelator with the at least one fluorocarbon.

Embodiment 18 relates to the method of Embodiment 17, wherein the at least one fluorocarbon forms a fluorous phase and the composition is a fluorous phase-encapsulated at least one fluorous hydroxamic acid chelator.

Embodiment 19 relates to the method of Embodiment 17, further comprising emulsifying the (i) at least one fluorocarbon and (ii) at least one fluorous hydroxamic acid chelator with at least one surfactant to obtain an emulsified composition.

Embodiment 20 relates to the method of Embodiment 19, wherein the at least one surfactant is at least one of egg lecithin, soybean lecithin, sunflower oil, 1,2-distearoyl-sn-glycero-3-phosphorylethanolamine (DSPE), DSPE-PEG2000, 1,2-distearoyl-sn-glycero-3-phosphoethanolamine-N-[methoxy(polyethylene glycol)-2000, DSPE-PEG3000, DSPE-PEG5000, DPPE, DPPE-PEG, cholesterol, Kolliphor EL, mannitol, CH₃—(CH₂)₅—(CF₂)₅—CF₃, Zonyl FS-300, Pluronic F68, Pluronic F127, Tween 20, and Tween 80.

Embodiment 21 relates to the method of Embodiment 19 or 20, wherein the at least one surfactant is at least one of egg lecithin, DSPE-PEG2000, and cholesterol.

Embodiment 22 relates to the method of Embodiments 19-21, further comprising radiolabeling the emulsified composition with at least one radioisotope to give a radiolabeled composition.

Embodiment 23 relates to the method of Embodiment 22, further comprising purifying the radiolabeled composition to give a purified composition.

Embodiment 24 relates to the method of Embodiment 22 or 23, further comprising formulating the radiolabeled composition or the purified composition in a buffer.

Embodiment 25 relates to the method of Embodiment 24, wherein the buffer is suitable for parenteral administration.

Embodiment 26 relates to the method of Embodiments 19-25, wherein the emulsified composition comprises droplets having a diameter from about 5 nm to about 500 nm as determined by dynamic light scattering (DLS).

Embodiment 27 relates to the method of Embodiments 22-25, wherein the at least one radioisotope is at least one of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺, and ⁸⁶Y³⁺.

Embodiment 28 relates to the method of Embodiments 22-27, wherein the at least one radioisotope is ⁸⁹Zr⁴⁺.

Embodiment 29 relates to the method of Embodiments 17-28, wherein the at least one fluorocarbon is at least one of:

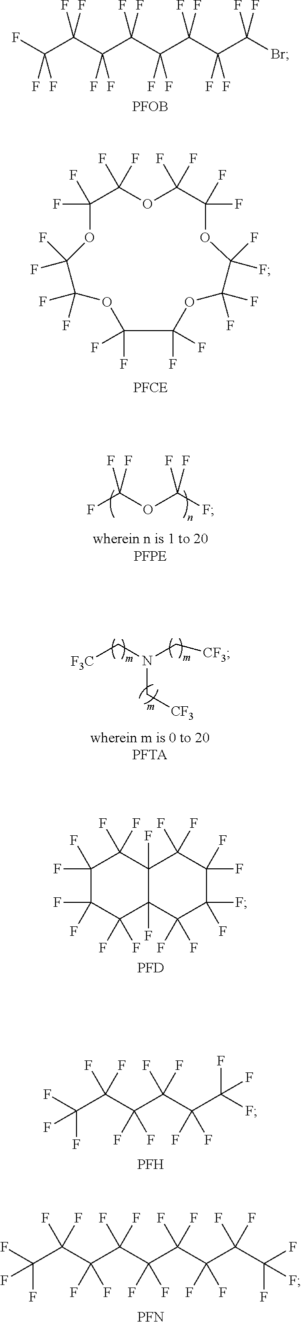

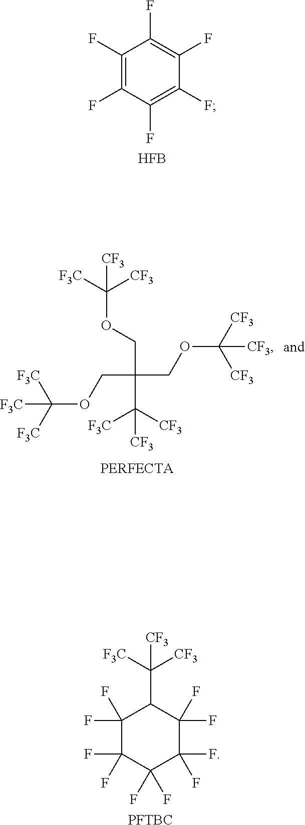

Embodiment 30 relates to the method of Embodiments 17-29, wherein the at least one fluorous hydroxamic acid chelator is at least one of:

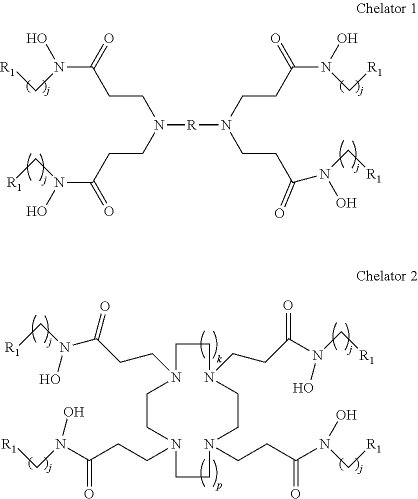

wherein R is a linker; each j is independently 1 to 20; k and p are each independently 0 to 3; each R₁ is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein each p and q is independently 0 to 20;

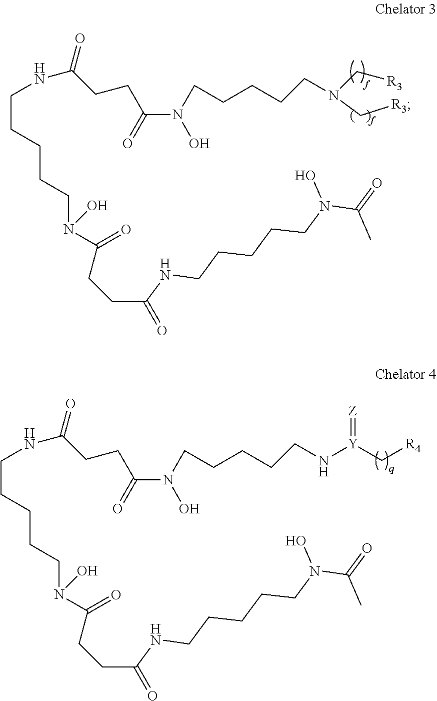

wherein each f and q is independently from 0 to 20; Y and Z are each selected from the group consisting of C, N, O, Si, P, and S; each R₃ and R₄ is independently selected from the group consisting of —(CF₂)ₚ—CF₃, —O(CF₂)ₚ—CF₃, —O—CF₂—(OCF₂CF₂)ₚ—OCF₃ and —[(CH₂)_{q}(CF₂)ₚCF₃]₂, wherein p and q are 0 to 20; and

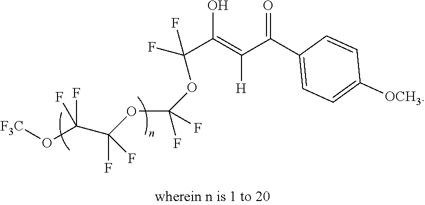

Embodiment 31 relates to the method of Embodiment 30, wherein Y and N form the groups C═O, C═S or P═O.

Embodiment 32 relates to the method of Embodiments 17-31, wherein the at least one fluorous hydroxamic acid chelator is at least one of:

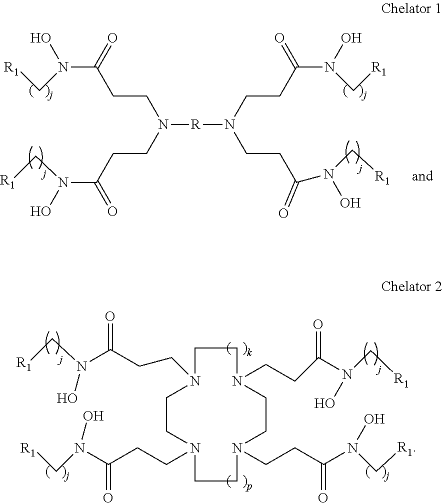

Embodiment 33 relates to the method of Embodiments 30-32, wherein R is a C₃-C₆-alkyl linker.

Embodiment 34 relates to the method of Embodiments 17-33, wherein the at least one fluorous hydroxamic acid chelator is at least one of:

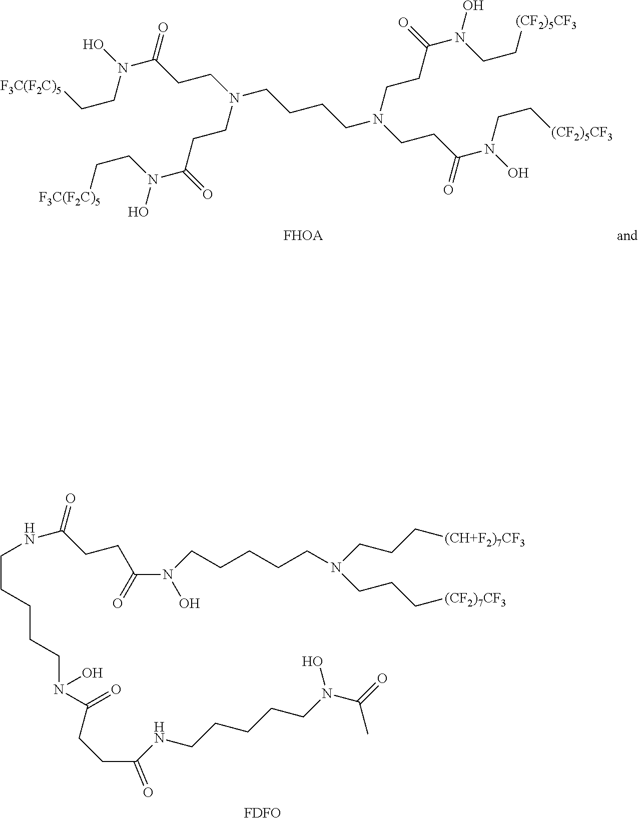

Embodiment 35 relates to a method for medical imaging comprising administering to a subject a composition comprising:
at least one fluorocarbon,
at least one fluorous hydroxamic acid chelator, and
at least one radioisotope; and
visualizing the administered composition in the subject.

Embodiment 36 relates to the method of Embodiment 35, wherein the visualizing is performed using at least one of PET, 1H MRI, FMRI, CT, SPECT, fluorescence imaging, and luminescent imaging.

Embodiment 37 relates to the method of Embodiment 35 or 36, wherein the medical imaging is imaging of inflammatory diseases.

Embodiment 38 relates to the method of Embodiment 35 or 36, wherein the medical imaging is imaging cancer and cancer metastasis.

Embodiment 39 relates to a method for cell tracking comprising administering to a subject a composition comprising:
at least one fluorocarbon,
at least one fluorous hydroxamic acid chelator, and
at least one radioisotope; and
visualizing the administered composition in the subject.

Embodiment 40 relates to the method of Embodiment 35-39, wherein the at least one fluorocarbon forms a fluorous phase and the composition is a fluorous phase-encapsulated at least one fluorous hydroxamic acid chelator.

Embodiment 41 relates to the method of Embodiments 35-39, wherein the composition is an emulsion.

Embodiment 42 relates to the method of Embodiment 41, wherein the emulsion comprises droplets having a diameter from about 5 nm to about 500 nm as determined by dynamic light scattering (DLS).

Embodiment 43 relates to the method of Embodiments 35-42, wherein the at least one radioisotope is at least one of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺, and ⁸⁶Y³⁺.

Embodiment 44 relates to the method of Embodiments 35-43, wherein the at least one radioisotope is ⁸⁹Zr⁴⁺.

Embodiment 45 relates to the method of Embodiments 35-44, wherein the at least one fluorocarbon is at least one of:

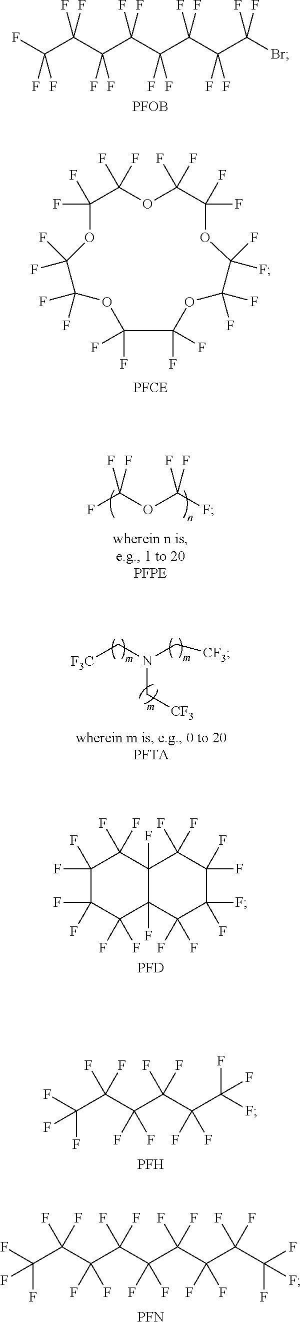

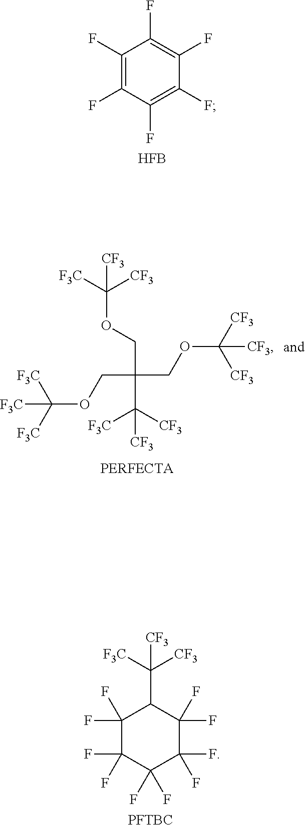

Embodiment 46 relates to the method of Embodiments 35-45, wherein the at least one fluorous hydroxamic acid chelator is at least one of:

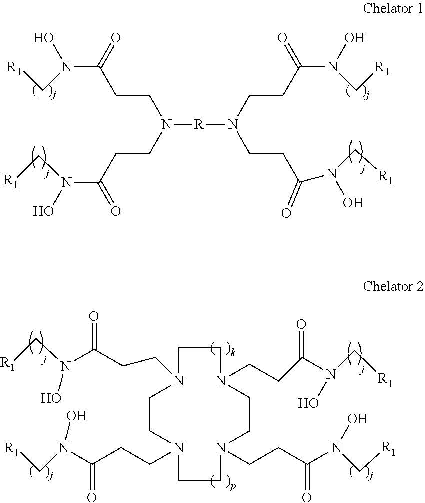

wherein R is a linker; each j is independently 1 to 20; k and p are each independently 0 to 3; each R, is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein each p and q is independently 0 to 20;

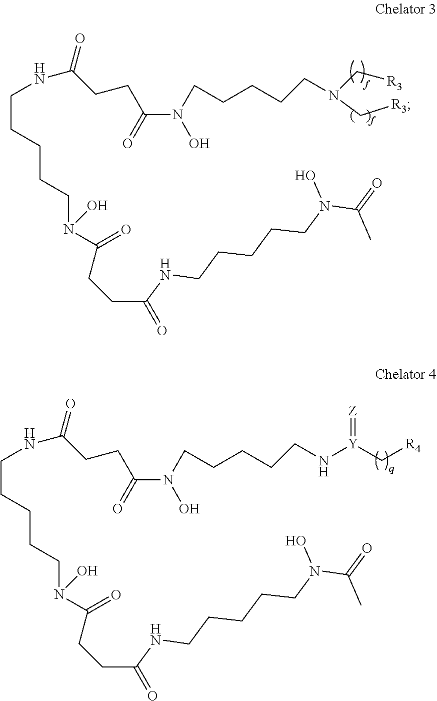

wherein each f and q is independently from 0 to 20; Y and Z are each selected from the group consisting of C, N, O, Si, P, and S; each R₃ and R₄ is independently selected from the group consisting of —(CF₂)ₚ—CF₃, —O—(CF₂)ₚ—CF₃, —O—CF₂—(OCF₂CF₂)ₚ—OCF₃ and —[(CH₂)_{q}(CF₂)ₚCF₃]₂, wherein p and q are 0 to 20; and

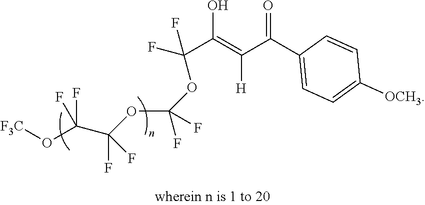

Embodiment 47 relates to the method of Embodiment 46, wherein Y and N form the groups C═O, C═S or P═O.

Embodiment 48 relates to the method of Embodiments 35-47, wherein the at least one fluorous hydroxamic acid chelator is at least one of:

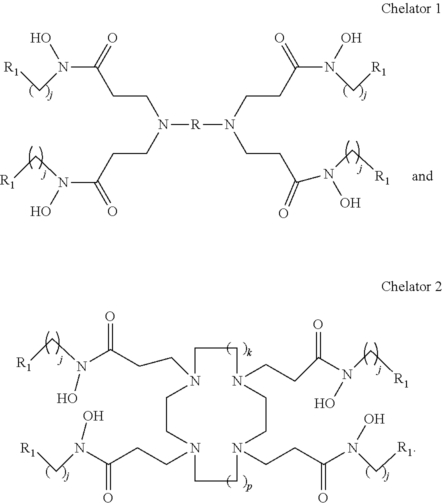

Embodiment 49 relates to the method of Embodiments 46-48, wherein R is a C₃-C₆-alkyl linker.

Embodiment 50 relates to the method of Embodiments 35-49, wherein the at least one fluorous hydroxamic acid chelator is at least one of:

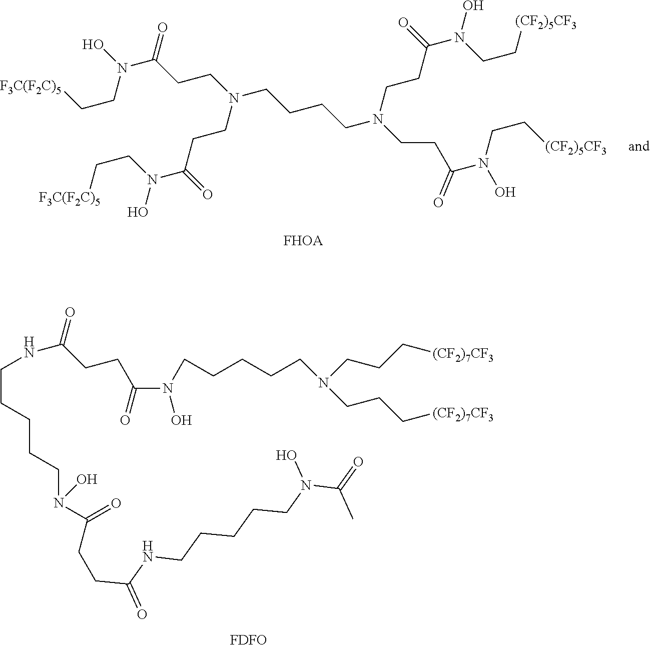

Embodiment 51 relates to a method of imaging inflammatory diseases, the method comprising:
(1) preparing or providing a composition comprising (i) at least one fluorocarbon, (ii) at least one fluorous hydroxamic acid chelator, and (iii) at least one radioisotope;
(2) optionally purifying, diluting or pH adjusting of the composition in (1);
(3) administering the composition from (1) or (2) to a subject;
(4) imaging of the subject in (3) after administration of the compositions from (1) or (2) to obtain acquired images; and
(5) optionally analyzing of the acquired images from (4).

Embodiment 52 relates to the method of Embodiment 51, wherein the acquired images identify the location of inflammation in the subject.

Embodiment 53 relates to the method of Embodiment 51 or 52, wherein the acquired images evaluate the severity or stages of inflammation.

Embodiment 54 relates to a method of imaging cancer and its metastasis, the method comprising:
(1) preparing or providing a composition comprising (i) at least one fluorocarbon, (ii) at least one fluorous hydroxamic acid chelator, and (iii) at least one radioisotope;
(2) optionally purifying, diluting or pH adjusting of the composition in (1);
(3) administering the composition from (1) or (2) to a subject;
(4) imaging of the subject in (3) after administration of the compositions from (1) or (2) to obtain acquired images; and
(5) optionally analyzing of acquired images from (4).

Embodiment 55 relates to the method of Embodiment 54, wherein the images can be acquired at different timepoints following the administering to monitor the development and spread of cancer. In addition, or alternatively, acquired images can be quantified to evaluate the stage and progression of cancer.

Embodiment 56 relates to a method of radiotherapy comprising administering a composition comprising (i) at least one fluorocarbon, (ii) at least one fluorous hydroxamic acid chelator, and (iii) at least one radioisotope to a subject.

Embodiment 57 relates to the method of Embodiments 51-56, wherein the radioisotope is selected from the group consisting of ⁹⁰Y, ⁶⁰Co, ¹³⁷Cs, ¹⁹²Ir, ²²⁶Ra, ¹⁷⁷Lu, and ¹⁵³Sm.

Embodiment 58 relates to the method of Embodiment 56 or 57, wherein the radiotherapy treats cancer.

Embodiment 59 relates to a theragnostic agent comprising (i) at least one fluorocarbon, (ii) at least one fluorous hydroxamic acid chelator, and (iii) at least one radioisotope, wherein the radioisotope is at least one of ⁸⁹Zr⁴⁺, ^{99m}Tc⁴⁺, ⁵⁹Fe³⁺, ⁶⁰Cu²⁺, ⁶¹Cu²⁺, ⁶²Cu²⁺, ⁶⁴Cu²⁺, ⁶⁷Cu²⁺, ⁶⁶Ga³⁺, ⁶⁷Ga³⁺, ⁶⁸Ga³⁺, ⁵²Mn²⁺, ⁸²Rb¹⁺, ¹¹¹In³⁺, ¹⁷⁷Lu³⁺, ⁴⁴Sc³⁺, ⁸⁶Y³⁺, ⁹⁰Y, ⁶⁰Co, ¹³⁷Cs, ¹⁹²Ir, ²²⁶Ra, ¹⁷⁷Lu, and ¹⁵³Sm.

Embodiment 60 relates to a method of diagnosing and treating a disease comprising administering the theragnostic agent of Embodiment 59 to a subject in need thereof.

Embodiment 61 relates to the method of Embodiment 60, wherein the disease is at least one of inflammation and cancer.

Embodiment 62 relates to a method for cell tracking, the method comprising:
(1) labeling cells with a compound comprising (i) at least one fluorocarbon, (ii) at least one fluorous hydroxamic acid chelator, and (iii) at least one radioisotope;
(2) optionally washing the labelled cells;
(3) administering of the labelled cells from (1) or (2) to a subject; and
(4) tracking a biodistribution of administered cells in the subject by acquiring images using imaging methods.

Embodiment 63 relates to the method of Embodiment 62, wherein the imaging method is at least one of PET, SPECT, MRI, and FMRI.

Embodiment 64 relates to the method of Embodiment 62 or 63, wherein the images are acquired at multiple timepoints after administration to assess the migration behavior of the cells.

1-64. (canceled)

65. A composition comprising:
(i) at least one fluorocarbon; and
(ii) at least one fluorous hydroxamic acid chelator,
wherein the at least one fluorous hydroxamic acid chelator is at least partially soluble in the fluorocarbon.

66. The composition of claim 65, wherein the at least one fluorocarbon forms a fluorous phase and the composition is a fluorous phase-encapsulated at least one fluorous hydroxamic acid chelator.

67. The composition of claim 65, wherein the composition is an emulsion.

68. The composition of claim 65, further comprising (iii) at least one radioisotope chelated by the at least one fluorous hydroxamic acid chelator.

69. The composition of claim 65, wherein the at least one fluorocarbon is at least one of:

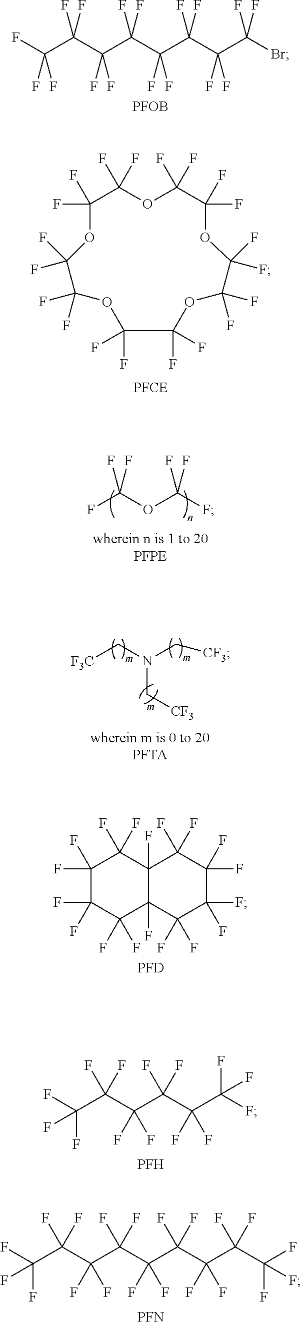

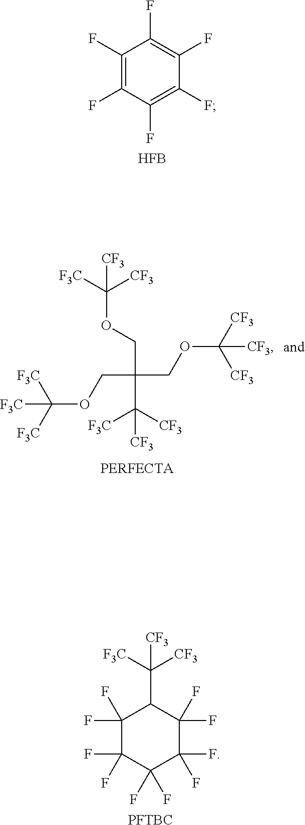

70. The composition of claim 65, wherein the at least one fluorous hydroxamic acid chelator is at least one of: 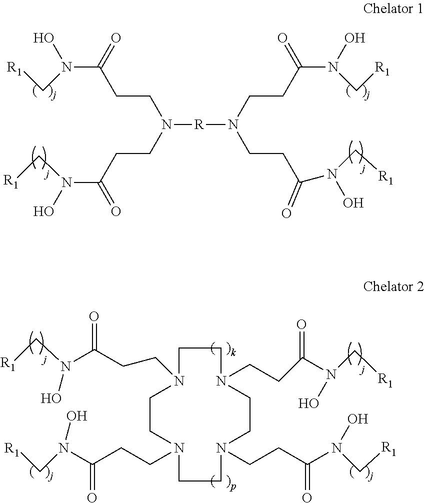
wherein R is a linker; each j is independently 1 to 20; k and p are each independently 0 to 3; each R₁ is independently selected from the group consisting of —(CF₂)ₚ—CF₃, —O—(CF₂)ₚ—CF₃, —O—CF₂—(OCF₂CF₂)ₚ—OCF₃ and
—[(CH₂)_{q}(CF₂)ₚCF₃]₂, wherein each n and q is independently 0 to 20; 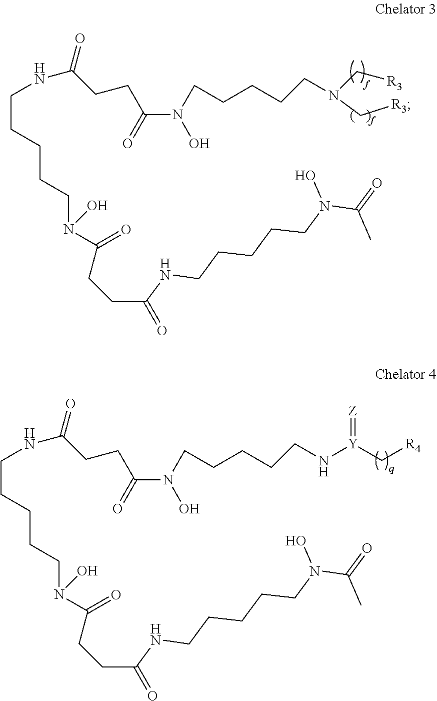
wherein each f and q is independently from 0 to 20; Y and Z are each selected from the group consisting of C, N, O, Si, P, and S; each R₃ and R₄ is independently selected from the group consisting of —(CF₂)ₙ—CF₃, —O—(CF₂)ₙ—CF₃, —O—CF₂—(OCF₂CF₂)ₙ—OCF₃ and —[(CH₂)ₘ(CF₂)ₙCF₃]₂, wherein p and q are 0 to 20; and

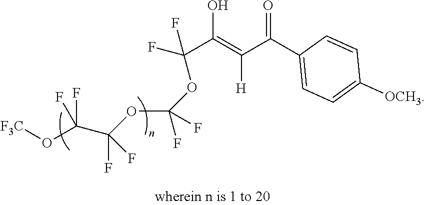

71. The composition of claim 65, wherein the at least one fluorous hydroxamic acid chelator is at least one of:

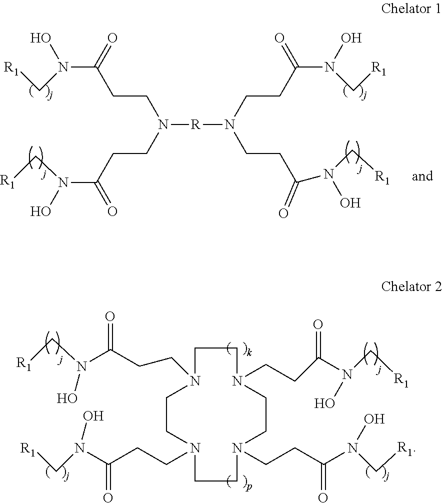

72. The composition of claim 65, wherein the at least one fluorous hydroxamic acid chelator is at least one of:

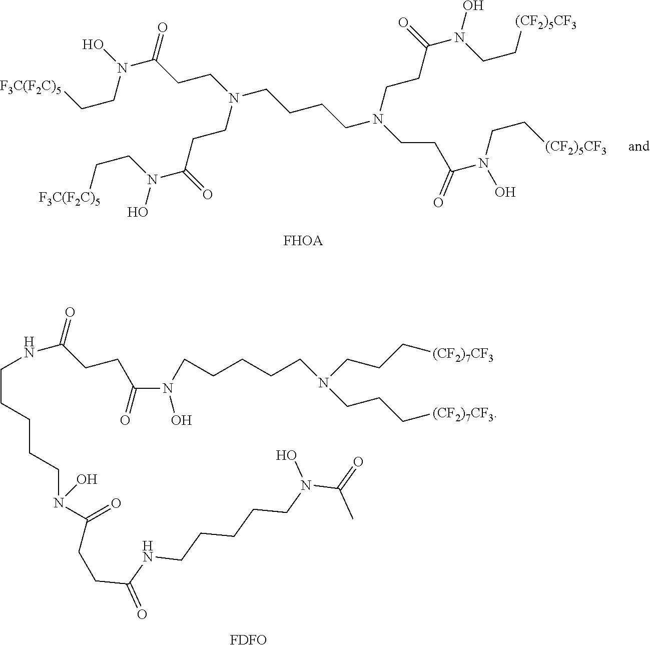

73. The composition of claim 65 further comprising at least one surfactant.

74. A method of making a composition of claim 65, the method comprising:
contacting the at least one fluorous hydroxamic acid chelator with the at least one fluorocarbon.

75. A method for imaging comprising administering to a subject a composition of claim 68, the method comprising:
visualizing the administered composition in the subject using at least one of PET, ¹H MRI, ¹⁹F MRI, CT, SPECT, fluorescence imaging, and luminescent imaging.

76. A method for cell tracking comprising administering to a subject a composition of claim 68, the method comprising:
visualizing the administered composition in the subject.

77. A method of imaging inflammatory diseases, the method comprising:
(1) preparing or providing a composition of claim 68;
(2) optionally purifying, diluting or pH adjusting of the composition in (1);
(3) administering the composition from (1) or (2) to a subject;
(4) imaging of the subject in (3) after administration of the compositions from (1) or (2) to obtain acquired images; and
(5) optionally analyzing of the acquired images from (4).

78. A method of imaging cancer and its metastasis, the method comprising:
(1) preparing or providing a composition of claim 68;
(2) optionally purifying, diluting or pH adjusting of the composition in (1);
(3) administering the composition from (1) or (2) to a subject;
(4) imaging of the subject in (3) after administration of the compositions from (1) or (2) to obtain acquired images; and
(5) optionally analyzing of acquired images from (4).

79. A method for cell tracking, the method comprising:
(1) labeling cells ex vivo with a compound of claim 68;
(2) optionally washing the labelled cells;
(3) administering of the labelled cells from (1) or (2) to a subject; and
(4) tracking a biodistribution of administered cells in the subject by acquiring images using imaging methods PET, SPECT and/or ¹⁹F MRI.
